(19) United States
(12) Patent Application Publication
(10) Pub. No.: US 2013/0344048 A1
(43) Pub. Date: Dec. 26, 2013

(54) Method of reducing insulin resistance by administering a Hyaluronan-degrading enzyme

(71) Applicants: David H. Wasserman, Nashville, TN (US); Li Kang, Nashville, TN (US)

(72) Inventors: David H. Wasserman, Nashville, TN (US); Li Kang, Nashville, TN (US)

(21) Appl. No.: 13/507,376

(22) Filed: Jun. 21, 2012

(51) Int. Cl. A61K 38/47; A61P 3/04; A61P 3/06; A61P 15/00; A61P 9/10; A61P 9/00; A61P 9/12; A61P 3/08; A61P 3/10

(52) U.S. Cl. 424/94.3

(57) ABSTRACT

Provided herein are methods of reducing or ameliorating insulin resistant using a hyaluronan-degrading enzyme, and in particular a hyaluronan-degrading enzyme that is conjugated to a polymer. The methods also can be used to prevent or ameliorate diseases and conditions associated with insulin resistance, such as cardiovascular disease and type 2 diabetes.

### INCORPORATION BY REFERENCE OF SEQUENCE LISTING PROVIDED ON COMPACT DISCS

An electronic version on compact disc (CD-R) of the Sequence Listing is filed herewith in duplicate (labeled Copy #1 and Copy #2), the contents of which are incorporated by reference in their entirety. The computer-readable file on each of the aforementioned compact discs, created on Jun. 21, 2012, is identical, 876 kilobytes in size, and titled 3103SEQ.001.txt.

### FIELD OF THE INVENTION

Provided herein are methods of reducing or ameliorating insulin resistance using a hyaluronan-degrading enzyme, and in particular a hyaluronan-degrading enzyme that is conjugated to a polymer. The methods also can be used to prevent or ameliorate diseases and conditions associated with insulin resistance, such as cardiovascular disease and type 2 diabetes.

### BACKGROUND

Insulin resistance is a condition in which muscle, fat and liver cells do not properly respond to insulin, resulting in a reduced uptake of glucose by the cells. As a result, the pancreas compensates by producing more insulin. This can result in hyperinsulinemia and other conditions that eventually can result in cardiovascular disease or type 2 diabetes. There is an estimated 3-16% of the population that has diagnosed insulin resistance, with an increased rate of occurrence in overweight or obese individuals. Currently, the primary treatment for insulin resistance and diseases or conditions associated with insulin resistance is diet and exercise. However, compliance for behavioral interventions is low. Hence, there is a need for alternative treatments for insulin resistance and for diseases and conditions associated with insulin resistance, such as type 2 diabetes and cardiovascular disease.

### SUMMARY

Provided herein are methods in which a hyaluronan-degrading enzyme is administered to a subject to reduce or ameliorate insulin resistance. For example, provided herein are methods to reduce or ameliorate insulin resistance by administering a hyaluronan-degrading enzyme conjugated to a polymer to a subject that exhibits a symptom or symptoms of insulin resistance. The administered hyaluronan-degrading enzyme is administered in an amount sufficient to remove or degrade skeletal muscle-associated hyaluronan, resulting in a decrease or elimination of insulin resistance.

In any of the methods provided herein or combinations thereof, the hyaluronan-degrading enzyme can be administered a plurality of times. For example, the hyaluronan-degrading enzyme can be administered to the subject more than once and at a frequency of at least once a month. In some examples, the hyaluronan-degrading enzyme is administered at a frequency of at least twice a month, at least once a week, at least twice a week, at least three times a week, at least four times a week, at least five times a week, at least six times a week or at least seven times a week. In one example, the hyaluronan-degrading enzyme is administered at a frequency of at least twice a week.

In any of the methods provided herein or combinations thereof, the hyaluronan-degrading enzyme can be administered a plurality of times at a predetermined frequency. The frequency of administration can be for a predetermined time in a cycle of administration. For example, the predetermined time can be at least or is one week, two weeks, three weeks, four weeks, two months, three months, four months, five months, six months, seven months, eight months, nine months, 10 months, 11 months or 12 months. The cycle of administration can be repeated a plurality of times. For example, in one example, the hyaluronan-degrading enzyme is administered at least once a month (the frequency) for at least 12 months (the predetermined time). In such an example, the cycle of administration of administering the hyaluronan-degrading enzyme at least once a month for at least 12 months can be repeated a plurality of times. The particular combination of frequency and time of treatment can be determined by a skilled physician, and can be dependent on the extent of the insulin resistance, the particular patient and other factors.

For example, in any of the methods provided herein or combinations thereof, the hyaluronan-degrading enzyme can be administered until a symptom of insulin resistance is reduced. For example, the hyaluronan-degrading enzyme can be administered until a symptom of insulin resistance is reduced by at least or about at least 5%, 10%, 20%, 30%, 40%, 50%, 60%, 70%, 80% or more compared to the symptom prior to treatment.

In examples of any of the methods provided herein or combinations thereof of administering a hyaluronan-degrading enzyme to a subject that exhibits a symptom or symptoms of insulin resistance, the subject is obese. For example, the subject can have a body mass index (BMI) of greater than 32 kg/m², 33 kg/m², 34 kg/m², 35 kg/m², 40 kg/m², 45 kg/m² or greater, or the subject can have a BMI of between or about between 30 kg/m² to 50 kg/m²; 30 kg/m² to 40 kg/m²; 35 kg/m² to 50 kg/m²; 30 kg/m² to 35 kg/m²; or 35 kg/m² to 40 kg/m². In one example, the subject has a BMI of greater than 30 kg/m². In other examples, a hyaluronan-degrading enzyme is administered to a subject that exhibits hyperglycemia, dyslipidemia or hyperlipidemia and/or hyperinsulinemia.

In examples of any of the methods provided herein or combinations thereof of administering a hyaluronan-degrading enzyme to a subject that exhibits a symptom or symptoms of insulin resistance, the insulin resistance can be diagnosed, assessed or determined by standard assays known to one of skill in the art. For example, insulin resistance can be assessed by, for example, determining fasting insulin levels, a glucose tolerance test (GTT), a hyperinsulinemic euglycemic clamp, an insulin tolerance test (ITT), an insulin sensitivity test (IST), continuous infusion of glucose with model assessment (CIGMA), homeostatic model assessment (HOMA-IR), quantitative insulin sensitivity check index (QUICKI), McAuley's index, Matsuda index, Belfiore index, Cederholm index, Gutt index, Avignon index and Stumboll index.

For example, in examples of any of the methods provided herein or combinations thereof, insulin resistance is assessed in a subject based on the fasting insulin levels. For example, a subject exhibits insulin resistance when the subject's fasting insulin levels are greater than 10 Units/mL, 15 Units/mL, 20 Units/mL, 25 Units/mL, 30 Units/mL, 35 Units/mL, 40 Units/mL or higher. In other examples, insulin resistance in a subject is determined by a homeostasis model assessment (HOMA-IR). A subject exhibits insulin resistance when the HOMA-IR value is greater than 2.2, 2.3, 2.5, 2.6, 2.7, 2.8, 2.9, 3.0, 3.5, 4.0, 5.0, 6.0 or greater than 6.0. In yet other examples, insulin resistance in a subject is assessed by a hyperinsulinemic euglycemic clamp. A subject exhibits insulin resistance when the glucose disposal rate (GDR), as assessed by a hyperinsulinemic euglycemic clamp is less than 7.5 mg/kg per minute, less than 7.0 mg/kg per minute, less than 6.5 mg/kg per minute, less than 6.0 mg/kg per minute, less than 5.5 mg/kg per minute, less than 5.0 mg/kg per minute, less than 4.5 mg/kg per minute, less than 4.0 mg/kg per minute, less than 3.5 mg/kg per minute, less than 3.0 mg/kg per minute or lower. In another example, insulin resistance in a subject is determined by a quantitative insulin sensitivity check (QUICKI). A subject exhibits insulin resistance when the QUICKI value is less than 0.360, 0.359, 0.358, 0.357, 0.355, 0.350, 0.345, 0.340, 0.335, 0.330, 0.325, 0.320, 0.315, 0.310 or less than 0.310. In other examples, insulin resistance in a subject is determined by McAuley's index. A subject exhibits insulin resistance when the McAuley's index value is less than 6.0, 5.9, 5.8, 5.7, 5.6, 5.5, 5.0, 4.5, 4.0, 3.5, 3.0 or less than 3.0.

In any of the methods herein or combinations thereof of administering a hyaluronan-degrading enzyme to a subject to reduce or ameliorate insulin resistance, prior to administering the hyaluronan-degrading enzyme, a subject is selected that exhibits a symptom or symptoms of insulin resistance or that exhibits increased or accumulated hyaluronan (HA) levels. For example, the subject can be one that exhibits increased or accumulated skeletal muscle-associated hyaluronan. In any of the methods provided herein, the subject is selected by measuring the expression or level of a hyaluronan-associated marker in a sample from a subject and comparing the expression or level of the marker to the expression or level of the same marker in a control sample or standard. The hyaluronan-associated marker measured in the any of the methods provided herein can be, for example, hyaluronan (HA), a hyaluronidase, a hyaluronan synthase, interstitial fluid pressure, vascular volume and water content. In instances where the marker is a hyaluronan, the hyaluronan can be detected using, for example, an anti-HA antibody or an HA-binding protein. In some examples, the marker is a hyaluronan synthase, such as hyaluronan synthase 2 (HAS2). If the marker is changed compared to the control sample or standard, the subject is selected for treatment with the hyaluronan-degrading enzyme. In some examples the expression or level of the marker is elevated or increased compared to the control sample or standard. For example, the expression or level of the marker can be elevated or increased at least 0.5-fold, 1-fold, 2-fold, 2.5-fold, 3.0-fold, 4.0-fold, 5.0-fold or greater compared to the control sample or standard. In other examples, the expression or level of the marker is decreased compared to the control sample or standard. For example, the expression or level of the marker can be decreased at least 0.5-fold, 1-fold, 2-fold, 2.5-fold, 3.0-fold, 4.0-fold, 5.0-fold or greater compared to the control sample or standard.

In examples of any of the methods provided herein or combinations thereof that include selecting a subject by measuring the expression or level of a hyaluronan-associated marker in a sample in the subject, the sample obtained from the subject can be a fluid or tissue sample. For example, in examples where the sample is a fluid sample, it can be a blood (plasma), urine or saliva sample. In other examples of any of the methods provided herein, the sample can be a sample from skeletal muscle. In instances where the sample is a skeletal muscle sample, the sample can be obtained by biopsy from a muscle, such as from soleus, gastrocnemius and vastus lateralis. The control sample used for comparison to the sample obtained from the subject can be selected from among an analogous sample from another subject that is a normal subject or a subject known to express low hyaluronan in the sample, and a cell line.

In any of the methods provided herein or combination thereof of administering a hyaluronan-degrading enzyme to reduce insulin resistance, the subject can have a disease or condition associated with insulin resistance. The disease or condition associated with insulin resistance can be, for example, obesity, type 2 diabetes, hypertension, dyslipidemia, coronary artery disease, atherosclerosis and polycystic ovarian syndrome. Hence, in the methods provided herein, of reducing or ameliorating insulin resistance, the methods can prevent or ameliorate a disease or condition associated with insulin resistance.

In examples of any of the methods provided herein or combinations thereof, the hyaluronan-degrading enzyme is a hyaluronidase. The hyaluronidase can be, for example, a PH20 or truncated form thereof, such as a truncated form that lacks a C-terminal glycosylphosphatidylinositol (GPI) attachment site or a portion of the GPI attachment site. The PH20 can be a human or non-human PH20.

In any of the methods provided herein or combinations thereof, the hyaluronan-degrading enzyme is a C-terminal truncated PH20 that does not contain the full-length sequence of PH20, such as the full-length sequence set forth in SEQ ID NO:1 or a mature form thereof. The truncated PH20 can contain at least the sequence of amino acids 36-464 of SEQ ID NO:1, or can contain a sequence of amino acids that has at least 85% sequence identity to the sequence of amino acids that contains at least amino acids 36-464 of SEQ ID NO:1 and retains hyaluronidase activity. In examples where the hyaluronan-degrading enzyme is a truncated PH20, the amino acid sequence of the truncated PH20 has at least 86%, 87%, 88%, 89%, 90%, 91%, 92%, 93%, 94%, 95%, 96%, 97%, 98% or 99% sequence identity to a sequence of amino acids that contains at least amino acids 36-464 of SEQ ID NO:1 and retains hyaluronidase activity.

In examples of any of the methods provided herein, the PH20 has a sequence of amino acids that contains a C-terminal truncation of a full-length PH20 polypeptide. The C-terminal truncated PH20 polypeptides provided for use in the methods herein can be secreted from cells when expressed, are soluble and are neutral active. With respect to the full-length sequence of human PH20 set forth in SEQ ID NO:1, the C-terminal truncation can be after, for example, amino acid position 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO:1. Hence, the C-terminal truncated mutant can include a contiguous sequence of amino acids set forth in SEQ ID NO:1 (e.g. depending on the signal peptide or heterologous signal peptide used, beginning at amino acid 34, 35, 36, 37, 38, etc.) that has the sequence of amino acids with a C-terminal amino acid residue that is or is after 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO:1. or a variant thereof that exhibits at least 85% sequence identity to a sequence of amino acids that contains a C-terminal amino acid residue that is or is after amino acid position 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO:1 and retains hyaluronidase activity. For example, the PH20 can have an amino acid sequence that has at least 86%, 87%, 88%, 89%, 90%, 91%, 92%, 93%, 94%, 95%, 96%, 97%, 98% or 99% sequence identity to a sequence of amino acids that contains a C-terminal truncation after amino acid position 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO:1 and retains hyaluronidase activity.

In particular examples of any of the methods provided herein or combinations thereof, the hyaluronan-degrading enzyme that is conjugated to a polymer is a C-terminal truncated PH20 of the full-length sequence of human PH20 set forth in SEQ ID NO:1, and that is secreted when expressed from cells, soluble and/or neutral active. For example, the hyaluronan-degrading enzyme is a C-terminal truncated PH20 that has or consist of the sequence of amino acids set forth in any of SEQ ID NOS: 4-9, 47, 48, 150-170, 183-189, or a sequence of amino acids that is at least 85%, 86%, 87%, 88%, 89%, 90%, 91%, 92%, 93%, 94%, 95%, 96%, 97%, 98%, 99% or more sequence identity to any of SEQ ID NOS: 4-9, 47, 48, 150-170, 183-189.

In any of the methods provided herein or combinations thereof to reduce or ameliorate insulin resistance by administering a hyaluronan-degrading enzyme, the hyaluronan-degrading enzyme is conjugated to a polymer. The polymer can be, for example, a polyalkylene glycol, a dextran, a pullulan or a cellulose. In examples where the polymer is a polyalkylene glycol, the polyalkylene glycol is selected from among polyethylene glycols (PEG) or methoxypolyethylene glycols (mPEG). In examples where the polymer is a PEG, the PEG can be chosen from among a branched PEG or a linear PEG. In some examples, the polymer is produced by reaction with methoxy-poly(ethylene glycol)-succinimidyl butanoate (mPEG-SBA) (5 kDa); methoxy-poly(ethylene glycol)-succinimidyl butanoate (mPEG-SBA) (20 kDa); methoxy-poly(ethylene glycol)-succinimidyl butanoate (mPEG-SBA) (30 kDa); methoxy-poly(ethylene glycol)-succinimidyl α-methylbutanoate (mPEG-SMB) (20 kDa); methoxy-poly(ethylene glycol)-succinimidyl α-methylbutanoate (mPEG-SMB) (30 kDa); methoxy-poly(ethylene glycol)-butyraldehyde (mPEG-butyraldehyde) (30 kDa), methoxy-poly(ethylene glycol)-succinimidyl propionate (mPEG-SPA) (20 kDa); methoxy-poly(ethylene glycol)-succinimidyl propionate (mPEG-SPA) (30 kDa); (methoxy-poly(ethylene glycol))₂-N-hydroxysuccinimide ester (mPEG₂-NHS) (10 kDa branched); (methoxy-poly(ethylene glycol))₂-N-hydroxysuccinimide ester (mPEG₂-NHS) (20 kDa branched); (methoxy-poly(ethylene glycol))₂-N-hydroxysuccinimide ester (mPEG₂-NHS) (40 kDa branched); (methoxy-poly(ethylene glycol))₂-N-hydroxysuccinimide ester (mPEG₂-NHS) (60 kDa branched); biotin-poly(ethylene glycol)-N-hydroxysuccinimide ester (biotin-PEG-NHS) (5 kDa biotinylated); poly(ethylene glycol)-p-nitrophenyl carbonate (PEG-p-nitrophenyl-carbonate) (30 kDa); or poly(ethylene glycol)-priopionaldehyde (PEG-propionaldehyde) (30 kDa). In one example, the polymer is a PEG that has a molecular weight of 30 kilodaltons (kDa) or about 30 kDa.

In any of the methods provided herein or combinations thereof, the hyaluronan-degrading enzyme can be administered in a dosage range amount of between or about between 0.001 μg/kg to 25 mg/kg (of the subject), 0.01 μg/kg to 25 mg/kg, 0.5 μg/kg to 25 mg/kg, 0.5 μg/kg to 10 mg/kg, 0.5 μg/kg to 2.0 mg/kg, 0.02 μg/kg to 1.5 mg/kg, 0.01 μg/kg to 15 μg/kg, 0.05 μg/kg to 10 μg/kg, 0.75 μg/kg to 7.5 μg/kg, 1.0 μg/kg to 3.0 μg/kg or 0.1 μg/kg to 1 μg/kg. For example, the dosage range amount of the hyaluronan-degrading enzyme is between or about between 0.5 μg/kg to 2.0 mg/kg, 0.02 μg/kg to 1.5 mg/kg, 0.01 μg/kg to 15 μg/kg, 0.05 μg/kg to 10 μg/kg, 0.75 μg/kg to 7.5 μg/kg, 1.0 μg/kg to 3.0 μg/kg or 0.1 μg/kg to 1 μg/kg.

In other examples of any of the methods provided herein or combinations thereof, the hyaluronan-degrading enzyme is administered in a dosage range amount of between or about between 0.3 Unit/kg to 800,000 Units/kg (of the subject), 1 Unit/kg to 800,000 Units/kg, 10 to 800,000 Units/kg, 10 to 750,000 Units/kg, 10 to 700,000 Units/kg, 10 to 650,000 Units/kg, 10 to 600,000 Units/kg, 10 to 550,000 Units/kg, 10 to 500,000 Units/kg, 10 to 450,000 Units/kg, 10 to 400,000 Units/kg, 10 to 350,000 Units/kg, 10 to 320,000 Units/kg, 10 to 300,000 Units/kg, 10 to 280,000 Units/kg, 10 to 260,000 Units/kg, 10 to 240,000 Units/kg, 10 to 220,000 Units/kg, 10 to 200,000 Units/kg, 10 to 180,000 Units/kg, 10 to 160,000 Units/kg, 10 to 140,000 Units/kg, 10 to 120,000 Units/kg, 10 to 100,000 Units/kg, 10 to 80,000 Units/kg, 10 to 70,000 Units/kg, 10 to 60,000 Units/kg, 10 to 50,000 Units/kg, 10 to 40,000 Units/kg, 10 to 30,000 Units/kg, 10 to 20,000 Units/kg, 10 to 15,000 Units/kg, 10 to 12,800 Units/kg, 10 to 10,000 Units/kg, 10 to 9,000 Units/kg, 10 to 8,000 Units/kg, 10 to 7,000 Units/kg, 10 to 6,000 Units/kg, 10 to 5,000 Units/kg, 10 to 4,000 Units/kg, 10 to 3,000 Units/kg, 10 to 2,000 Units/kg, 10 to 1,000 Units/kg, 10 to 900 Units/kg, 10 to 800 Units/kg, 10 to 700 Units/kg, 10 to 500 Units/kg, 10 to 400 Units/kg, 10 to 300 Units/kg, 10 to 200 Units/kg, 10 to 100 Units/kg, 16 to 600,000 Units/kg, 16 to 500,000 Units/kg, 16 to 400,000 Units/kg, 16 to 350,000 Units/kg, 16 to 320,000 Units/kg, 16 to 160,000 Units/kg, 16 to 80,000 Units/kg, 16 to 40,000 Units/kg, 16 to 20,000 Units/kg, 16 to 16,000 Units/kg, 16 to 12,800 Units/kg, 16 to 10,000 Units/kg, 16 to 5,000 Units/kg, 16 to 4,000 Units/kg, 16 to 3,000 Units/kg, 16 to 2,000 Units/kg, 16 to 1,000 Units/kg, 16 to 900 Units/kg, 16 to 800 Units/kg, 16 to 700 Units/kg, 16 to 500 Units/kg, 16 to 400 Units/kg, 16 to 300 Units/kg, 16 to 200 Units/kg, 16 to 100 Units/kg, 160 to 12,800 Units/kg, 160 to 8,000 Units/kg, 160 to 6,000 Units/kg, 160 to 4,000 Units/kg, 160 to 2,000 Units/kg, 160 to 1,000 Units/kg, 160 to 500 Units/kg, 500 to 5000 Units/kg, 1000 to 100,000 Units/kg or 1000 to 10,000 Units/kg.

In any of the methods provided herein or combinations thereof, the hyaluronan-degrading enzyme can be administered orally, intravenously (IV), subcutaneously, intramuscularly, intra-tumorally, intradermally, topically, transdermally, rectally, intrathecally or sub-epidermally. In one example, the hyaluronan-degrading enzyme is administered intravenously, subcutaneously, or intramuscularly.

In some examples of any of the methods provided herein or combinations thereof, a corticosteroid is administered in addition to the hyaluronan-degrading enzyme. The corticosteroid can be, for example, a glucocorticoid. The glucocorticoid can be selected from among cortisones, dexamethasones, hydrocortisones, methylprednisolones, prednisolones and prednisones. In some examples, the corticosteroid is administered prior to, concurrent with, intermittently with, or subsequent to administration of the hyaluronan-degrading enzyme. In one example, the corticosteroid is administered with the hyaluronan-degrading enzyme. In another example, the corticosteroid is administered prior to the administration of the hyaluronan-degrading enzyme, for example, at least 1 hour prior to administration of the hyaluronan-degrading enzyme. In yet other examples, the corticosteroid is administered after administration of the hyaluronan-degrading enzyme, for example, at least 8 hours to 12 hours after administration of the hyaluronan-degrading enzyme.

In any of the examples provided herein in which a corticosteroid is administered in addition to the hyaluronan-degrading enzyme, the amount of corticosteroid administered is between or about between 0.1 to 20 mgs, 0.1 to 15 mgs, 0.1 to 10 mgs, 0.1 to 5 mgs, 0.2 to 20 mgs, 0.2 to 15 mgs, 0.2 to 10 mgs, 0.2 to 5 mgs, 0.4 to 20 mgs, 0.4 to 15 mgs, 0.4 to 10 mgs, 0.4 to 5 mgs, 0.4 to 4 mgs, 1 to 20 mgs, 1 to 15 mgs or 1 to 10 mgs. In the examples provided herein, the corticosteroid can be administered orally.

The any of the methods provided herein or combinations thereof of reducing or ameliorating insulin resistance, the hyaluronan-degrading enzyme can be administered in combination with another agent or another treatment for reducing or ameliorating insulin resistance. In some examples, the treatment is selected from among exercise or diet. In other examples, the agent or treatment is selected from among an insulin-sensitizing agent, for example, metformin, rosiglitazone, pioglitazone, troglitazone and D-chiro-inositol. In particular, in any of the examples provided herein, the additional agent or treatment is an agent or treatment for reducing or ameliorating a disease or condition associated with insulin resistance, for example, type 2 diabetes, cardiovascular disease or polycystic syndrome.

For example, in any of the examples provided herein, the subject that is treated has a disease or condition associated with insulin resistance that is type 2 diabetes, and a further agent or treatment of treating type 2 diabetes also is administered to the subject. The further treatment or agent can be selected from among a sulfonylurea, IGF-1, metformin, troglitazone, vandate or vanadium salts, phenyloin, sitagliptin, saxagliptin, repaglinide, nateglinide, Exenatide, liraglutide, and an insulin. In examples where the further treatment or agent is an insulin, the insulin can be, for example, a rapid-acting insulin analog or a super-fast-acting insulin composition. In some examples, the insulin is a rapid-acting insulin analog, and the rapid-acting insulin analog is selected from among insulin lispro, insulin aspart and insuline glulisine. In other examples, the insulin is a super-fast acting insulin composition, and the composition can contain a rapid-acting insulin analog and a hyaluronan-degrading enzyme.

In some examples of any of the methods provided herein, the subject that is treated has a disease or condition associated with insulin resistance that is a cardiovascular disease, and a further agent or treatment for treating cardiovascular disease is administered to the subject. The further treatment or agent can be a lipid-lowering drug or an anti-hypertensive agent. In examples where the further treatment or agent is a lipid-lowering drug, the lipid-lowering drug is selected from among a statin, a resin, a cholesterol absorption inhibitor, a fibrate or nicotinic acid. For example, the agent can be selected from among pravastatin, lovastatin, fluvastatin, simivastatin, rosuvastatin, L-Cholest, cholestyramine, colesevelam, colestipol, ezetimibe, simvastatin and ezetimibe combined, fenofibrate, bezabifrate, gemifibrozil and niacin. In examples where the agent is an anti-hypertensive agent, the agent is selected from among a renin inhibitor, an Angiotensin II receptor antagonist, an adrenergic receptor antagonist, a calcium channel blocker, a diuretic, an ACE inhibitor, an aldosterone antagonist, a vasodilators and an alpha-2 agonists. For example, the agent can be selected from among bumetanide, ethacrynic acid, furosemide, torsemide, epitizide, hydrochlorothiazide, chlorothiazide, bendroflumethiazide, indapamide, chlorthalidone, metolazone, amiloride, triamterene, spironolactone, atenolol, metoprolol, nadolol, oxprenolol, pindolol, propranolol, timolol, doxazosin, phentolamine, indoramin, phenoxybenzamine, prazosin, terazosin, tolazoline, bucindolol, carvedilol, labetalol, amlodipine, felodipine, isradipine, lercanidipine, nicardipine, nifedipine, nimodipine, nitrendipine, diltiazem, verapamil, captopril, enalapril, fosinopril, lisinopril, perindopril, quinapril, ramipril, trandolapril, benazepril, candesartan, eprosartan, irbesartan, losartan, olmesartan, telmisartan, valsartan, eplerenone, spironolactone, sodium nitroprusside, hydralazine, clonidine, guanabenz, methyldopa, moxonidine, guanethidine, and reserpine.

In some examples of any of the methods provided herein, the subject that is treated has a disease or condition associated with insulin resistance that is a polycystic ovary syndrome, and a further agent or treatment for treating polycystic ovary syndrome is administered to the subject. The further treatment or agent can be selected from among an ovulation medication, an anti-androgen, ovarian drilling surgery and in vitro fertilization (IVF).

In any of the methods provided herein above or combinations thereof, the subject used in the methods provided herein can be a human subject.

### DETAILED DESCRIPTION

### Outline

A. Definitions

B. Insulin Resistance and Hyaluronan

C. Hyaluronan-Degrading Enzymes and Polymer-Conjugated Hyaluronan-Degrading Enzymes
1. Hyaluronidases
   b. Mammalian-type hyaluronidases
      PH20
   c. Bacterial Hyaluronidase
   d. Hyaluronidases from leeches, other parasites and crustaceans
2. Other hyaluronan-degrading enzymes
3. Soluble hyaluronan-degrading enzymes
   a. Soluble Human PH20
   b. rHuPH20
4. Glycosylation of hyaluronan-degrading enzymes
5. Modified (Polymer-Conjugated) Hyaluronan-degrading Enzymes PEGylated Soluble hyaluronan-degrading enzymes

D. Methods of Producing Nucleic Acids and Encoded Polypeptides of Hyaluronan-degrading Enzymes
1. Vectors and Cells
2. Expression
   a. Prokaryotic Cells
   b. Yeast Cells
   c. Insect Cells
   d. Mammalian cells
   e. Plants
3. Purification Techniques
4. PEGylation of Hyaluronan-degrading Enzyme Polypeptides

E. Pharmaceutical Compositions and Formulations
1. Formulations
   a. Injectables, solutions and emulsions
   b. Lyophilized powders
   c. Topical administration
   d. Compositions for other routes of administration
2. Formulation Amounts
3. Packaging and Articles of Manufacture

F. Methods of Assessing Activity, Bioavailability and Pharmacokinetics
1. In Vitro Assays
   a. Hyaluronidase Activity
   b. Insulin Action
2. In Vivo Animal Models
3. Ex vivo assays
3. Pharmacokinetics, pharmacodynamics and tolerability

G. Methods of Treatment and Uses

H. Examples

### A. DEFINITIONS

Unless defined otherwise, all technical and scientific terms used herein have the same meaning as is commonly understood by one of skill in the art to which the invention(s) belong. All patents, patent applications, published applications and publications, Genbank sequences, databases, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

As used herein, hyaluronan (HA or hyaluronic acid) refers to a glycosaminoglycan that is distributed throughout the connective, epithelial and neural tissues. It is a polymer of disaccharides, composed of D-glucuronic acid and D-N-acetylglucosamine, linked via alternating β-1,4 and β-1,3 glycosidic bonds, ranging in size from 5,000 to 20,000,000 Da. HA is a component of the extracellular matrix (ECM) and contributes to processes such as tissue hydrodynamics, movement and proliferation of cells and cell interactions through its primary receptors CD44 and RHAMM. HA is degraded by hyaluronan-degrading enzymes, such as hyaluronidases (e.g. PH20).

As used herein, “elevated” or “increased,” with reference to hyaluronan levels, refers to accumulated or higher amounts of hyaluronan in a particular tissue, body fluid or cell compared to other tissues, body fluids or cells. The reference levels on other tissues, body fluids or cells can be levels present in tissues, body fluids or cells of non-diseased or healthy or normal subjects. Hence, the elevated levels can be a consequence of, or otherwise observed in, various diseases or condition. For example, as shown herein, increased levels of hyaluronan in skeletal muscle is associated with insulin resistance.

As used herein, disease, disorder or condition are used interchangeably and refer to a pathological state in an organism or subject resulting from a physiologic defect or cause including, but not limited to, infections, acquired defects and genetic defects, and are characterized by identifiable symptoms. Diseases and conditions of interest herein are insulin resistance and diseases or conditions associated with insulin resistance.

As used herein, “insulin action” or “action of insulin” refers to the consequence of insulin binding to its plasma membrane receptor and effecting glucose uptake into muscle tissue and other tissues.

As used herein, “insulin resistance” refers to a condition characterized by a reduced response of cells to the action of insulin in transporting glucose from the bloodstream into muscle and other tissues, and an associated increased in the production of insulin by the pancreas, as compared to a normal population (i.e. a non-diabetic, non-obese population, generally having regular menstrual cycles for women, not suffering from hirsutism and having normal circulating androgen levels). Insulin resistance can lead to increased or elevated levels of insulin in the blood. In some cases, increased or elevated glucose levels also are present. Hence, insulin resistance is associated with hyperinsulinemia and can be associated with hyperglycemia. Insulin resistance is a component of or is associated with several disorders, including, but not limited to, type 2 diabetes; metabolic syndrome (Syndrome X); cardiovascular disease associated with hypertension, hyperlipidemia, or coronary artery disease; polycystic ovary syndrome; and ovarian hyperthecosis. Insulin resistance can be diagnosed or determined by various tests well-known to one of skill in the art and described herein, including but not limited to, fasting insulin levels, fasting glucose levels, hyperinsulinemic euglycemic clamp, homeostatic model assessment (HOMA), quantitative insulin sensitivity check index (QUICKI) and McAuley's index. The results of the test can be compared to a normal population or in a general population to determine the score, value or parameter indicative of subnormal insulin action in any of the available tests (see e.g. McAuley et al. (2001) Diabetes Care 24:460-464; Radikova (2003) Endocrine Regulations 37:189-194; Ascaso et al. (2003) Diabetes Care 26:3320-3325; Tam et al., Diabetes Care, published online Apr. 17, 2012 ahead of print). Generally, a level above the upper quartile in the fasting state in someone without diabetes is considered abnormal.

As used herein, a disease or condition associated with insulin resistance is any disease or condition in which insulin resistance is associated with the etiology, or is a cause, symptom or a factor in the disease or condition. Exemplary of diseases and conditions associated with insulin resistance include, but are not limited to, type 2 diabetes; metabolic syndrome (Syndrome X); cardiovascular disease associated with hypertension, hyperlipidemia, or coronary artery disease; polycystic ovary syndrome; and ovarian hyperthecosis.

As used herein, a symptom of insulin resistance refers to a sign or feature that is a characteristic of or that indicates that a subject is likely to exhibit insulin resistance and includes, but is not limited to, one or more features or characteristics selected from among fatigue, brain fogginess, intestinal bloating, sleepiness, increased triglycerides (e.g. dyslipidemia), increased blood pressure (hypertension), increased weight and fat storage and depression. In particular, symptoms of insulin resistance include hyperinsulinemia due to higher concentrations of insulin secreted by the pancreas. In some cases, low blood sugar (hypoglycemia) can be a feature of insulin resistance, particularly in pre-diabetic patients if their high circulating insulin levels are further challenged by a prolonged period of fasting. Insulin resistance also can be associated with hyperglycemia, in particular as the condition advances to type 2 diabetes. Obese persons typically have insulin resistance.

As used herein, hyperglycemia refers to a condition in which an excessive amount of glucose circulates in the blood. Signs and symptoms of hyperglycemia include, for example, high blood glucose, high levels of sugar in the urine, frequent urination and increased thirst. For example, a subject having a glucose level higher than 126 mg/dL or higher than 7 mmol/L exhibits hyperglycemia.

As used herein, hypoglycemia or low blood sugar refers to an abnormally diminished content of glucose in the blood.

As used herein, hyperinsulinemia refers to a condition in which there are excess levels of insulin circulating in the blood than expected relative to the level of glucose.

As used herein, blood pressure refers to a measure of the blood flow through the arteries measured in millimeters of mercury (mm Hg). Blood pressure includes the systolic pressure (the pressure when the heart is beating) and the diastolic pressure (the pressure when the heart is at rest). Blood pressure is generally depicted as the systolic pressure first and the diastolic pressure second.

As used herein, hypertension refers to high blood pressure. Blood pressure is high if it is greater than normal, which is defined as less than 120 over less than 80 (e.g. 120/80 mm Hg). Typically, pre-hypertension exists if blood pressure is between 120-139/80-89 mm Hg and hypertension exists if blood pressure is greater than or about or between 140-170 mm Hg/90-109 mm Hg, such as between or about between or greater than 140-159/90-99 mm Hg or 160-179/100-109 mm Hg.

As used herein, dyslipidemia refers to the presence of abnormal (e.g. high; hyperlipidemia) lipid levels, such as cholesterol and/or fat, in the blood. Dyslipidemia or hyperlipidemia is generally characterized by elevation of total cholesterol, the low-density lipoprotein (LDL) cholesterol and the triglyceride concentration. Generally, dyslipidemia is associated with decreased concentration of high-density lipoprotein (HDL) cholesterol in the blood.

As used herein, obesity refers to a condition characterized by an excess of body fat. Obesity can be measured based on body mass index (BMI), which is a measurement that considers weight and height. A person is generally considered to be overweight if their BMI is greater than 25 kg/m² (e.g. between 25 kg/m² to 30 kg/m²), and is severely obese with a BMI greater than 30 kg/m².

As used herein, reduction or amelioration of the symptoms of a particular disease or disorder by a treatment, such as by administration of a pharmaceutical composition or other therapeutic, refers to any lessening, whether permanent or temporary, lasting or transient, of the symptoms.

As used herein, reducing or ameliorating insulin resistance by an agent, such as a hyaluronan-degrading enzyme as provided herein, refers to a lessening, whether permanent or temporary, lasting or transient, of one or more symptoms of insulin resistance. For example, reducing or ameliorating insulin resistance can be evidenced by a decrease in weight, hypertension, triglyceride levels (dyslipidemia), hyperinsulinemia, or hyperglycemia. A symptom of insulin resistance is reduced if there is a decrease in the symptom by at least 5%, 10%, 15%, 20%, 30%, 40%, 50%, 60%, 70%, 80%, 90% or more compared to the level or extent of the symptom prior to the treatment or prior to the last administration of the treatment.

As used herein, prevention or prophylaxis refers to reduction in the risk of developing a disease or condition.

As used herein, the term “cardiovascular disease” is intended to include a range of symptoms, conditions, and/or diseases including atherosclerosis, coronary artery disease, pulmonary embolism, diabetic cardiomyopathy, angina pectoris, carotid artery disease, strokes, peripheral vascular disease, cerebral arteriosclerosis, myocardial infarction, high blood pressure, cerebral infarction, restenosis following balloon angioplasty, intermittent claudication, dyslipidemia post-prandial lipidemia and xanthoma, and all conventionally targeted symptoms arising from or associated with the foregoing diseases and conditions.

As used herein, type 2 diabetes (T2DM; also referred to as non-insulin-dependent diabetes mellitus (NIDDM) or adult-onset diabetes) refers to a metabolic disorder characterized by high blood glucose as a result of insulin resistance. The World Health Organization (WHO) characterizes diabetes, including T2DM, as fasting plasma glucose ≧7.0 mmol/L (126 mg/dL) or a 2 hour glucose tolerance test result after oral dose of a plasma glucose of ≧11.1 mmol/L (200 mg/dL). In addition, glycated hemoglobin (HbA_{1c}) also can be an indicator of diabetes, with measures of ≧6.5% HbA_{1c} indicative of diabetes.

As used herein, a hyaluronan-degrading enzyme refers to an enzyme that catalyzes the cleavage of a hyaluronan polymer (also referred to as hyaluronic acid or HA) into smaller molecular weight fragments. Exemplary of hyaluronan-degrading enzymes are hyaluronidases, and particular chondroitinases and lyases that have the ability to depolymerize hyaluronan. Exemplary chondroitinases that are hyaluronan-degrading enzymes include, but are not limited to, chondroitin ABC lyase (also known as chondroitinase ABC), chondroitin AC lyase (also known as chondroitin sulfate lyase or chondroitin sulfate eliminase) and chondroitin C lyase. Chondroitin ABC lyase includes two enzymes, chondroitin-sulfate-ABC endolyase (EC 4.2.2.20) and chondroitin-sulfate-ABC exolyase (EC 4.2.2.21). Exemplary chondroitin-sulfate-ABC endolyases and chondroitin-sulfate-ABC exolyases include, but are not limited to, those from Proteus vulgaris and Flavobacterium heparinum (the Proteus vulgaris chondroitin-sulfate-ABC endolyase is set forth in SEQ ID NO:98; Sato et al. (1994) Appl. Microbiol. Biotechnol. 41(1):39-46). Exemplary chondroitinase AC enzymes from the bacteria include, but are not limited to, those from Flavobacterium heparinum Victivallis vadensis, set forth in SEQ ID NO:99, and Arthrobacter aurescens (Tkalec et al. (2000) Appl. Environ. Microb. 66(1):29-35; Ernst et al. (1995) Crit. Rev. Biochem. Mol. Biol. 30(5):387-444). Exemplary chondroitinase C enzymes from the bacteria include, but are not limited to, those from Streptococcus and Flavobacterium (Hibi et al. (1989) FEMS-Microbiol-Lett. 48(2):121-4; Michelacci et al. (1976) J. Biol. Chem. 251:1154-8; Tsuda et al. (1999) Eur. J. Biochem. 262:127-133).

As used herein, hyaluronidase refers to a class of hyaluronan-degrading enzymes. Hyaluronidases include bacterial hyaluronidases (EC 4.2.2.1 or EC 4.2.99.1), hyaluronidases from leeches, other parasites, and crustaceans (EC 3.2.1.36), and mammalian-type hyaluronidases (EC 3.2.1.35). Hyaluronidases include any of non-human origin including, but not limited to, murine, canine, feline, leporine, avian, bovine, ovine, porcine, equine, piscine, ranine, bacterial, and any from leeches, other parasites, and crustaceans. Exemplary non-human hyaluronidases include, hyaluronidases from cows (SEQ ID NOS:10, 11, 64 and BH55 (U.S. Pat. Nos. 5,747,027 and 5,827,721)), yellow jacket wasp (SEQ ID NOS:12 and 13), honey bee (SEQ ID NO:14), white-face hornet (SEQ ID NO:15), paper wasp (SEQ ID NO:16), mouse (SEQ ID NOS:17-19, 32), pig (SEQ ID NOS:20-21), rat (SEQ ID NOS:22-24, 31), rabbit (SEQ ID NO:25), sheep (SEQ ID NOS:26, 27, 63 and 65), chimpanzee (SEQ ID NO:101), Rhesus monkey (SEQ ID NO:102), orangutan (SEQ ID NO:28), cynomolgus monkey (SEQ ID NO:29), guinea pig (SEQ ID NO:30), Arthrobacter sp. (strain FB24) (SEQ ID NO:67), Bdellovibrio bacteriovorus (SEQ ID NO:68), Propionibacterium acnes (SEQ ID NO:69), Streptococcus agalactiae ((SEQ ID NO:70); 18RS21 (SEQ ID NO:71); serotype 1a (SEQ ID NO:72); serotype III (SEQ ID NO:73)), Staphylococcus aureus (strain COL (SEQ ID NO:74); strain MRSA252 (SEQ ID NOS:75 and 76); strain MSSA476 (SEQ ID NO:77); strain NCTC 8325 (SEQ ID NO:78); strain bovine RF122 (SEQ ID NOS:79 and 80); strain USA300 (SEQ ID NO:81)), Streptococcus pneumoniae ((SEQ ID NO:82); strain ATCC BAA-255/R6 (SEQ ID NO:83); serotype 2, strain D39/NCTC 7466 (SEQ ID NO:84)), Streptococcus pyogenes (serotype M1 (SEQ ID NO:85); serotype M2, strain MGAS 10270 (SEQ ID NO:86); serotype M4, strain MGAS 10750 (SEQ ID NO:87); serotype M6 (SEQ ID NO:88); serotype M12, strain MGAS2096 (SEQ ID NOS:89 and 90); serotype M12, strain MGAS9429 (SEQ ID NO:91); serotype M28 (SEQ ID. NO:92)), Streptococcus suis (SEQ ID NOS:93-95), Vibrio fischeri (strain ATCC 700601/ES 114 (SEQ ID NO:96)), and the Streptomyces hyaluronolyticus hyaluronidase enzyme, which is specific for hyaluronic acid and does not cleave chondroitin or chondroitin sulfate (Ohya and Kaneko (1970) Biochim. Biophys. Acta 198:607). Hyaluronidases also include those of human origin. Exemplary human hyaluronidases include HYAL1 (SEQ ID NO:36), HYAL2 (SEQ ID NO:37), HYAL3 (SEQ ID NO:38), HYAL4 (SEQ ID NO:39), and PH20 (SEQ ID NO:1). Also included amongst hyaluronidases are soluble hyaluronidases, including ovine and bovine PH20, soluble human PH20 and soluble rHuPH20. Examples of commercially available bovine or ovine soluble hyaluronidases include Vitrase® (ovine hyaluronidase), Amphadase® (bovine hyaluronidase) and Hydase™ (bovine hyaluronidase).

As used herein, “purified bovine testicular hyaluronidase” refers to a bovine hyaluronidase purified from bovine testicular extracts (see U.S. Pat. Nos. 2,488,564; 2,488,565; 2,806,815; 2,808,362; 2,676,139; 2,795,529; 5,747,027; and 5,827,721). Examples of commercially available purified bovine testicular hyaluronidases include Amphadase® and Hydase™, and bovine hyaluronidases, including, but not limited to, those available from Sigma Aldrich, Abnova, EMD Chemicals, GenWay Biotech, Inc., Raybiotech, Inc., and Calzyme. Also included are recombinantly produced bovine hyaluronidases, such as but not limited to, those generated by expression of a nucleic acid molecule set forth in any of SEQ ID NOS:190-192.

As used herein, “purified ovine testicular hyaluronidase” refers to an ovine hyaluronidase purified from ovine testicular extracts (see U.S. Pat. Nos. 2,488,564; 2,488,565; and 2,806,815; and International PCT Application No. WO 2005/118799). Examples of commercially available purified ovine testicular extract include Vitrase®, and ovine hyaluronidases including, but not limited to, those available from Sigma Aldrich, Cell Sciences, EMD Chemicals, GenWay Biotech, Inc., Mybiosource.com and Raybiotech, Inc. Also included are recombinantly produced ovine hyaluronidases, such as, but not limited to, those generated by expression of a nucleic acid molecule set forth in any of SEQ ID NOS:66 and 193-194.

As used herein, “PH20” refers to a type of hyaluronidase that occurs in sperm and is neutral-active. PH20 occurs on the sperm surface, and in the lysosome-derived acrosome, where it is bound to the inner acrosomal membrane. PH20 includes those of any origin including, but not limited to, human, chimpanzee, cynomolgus monkey, rhesus monkey, murine, bovine, ovine, guinea pig, rabbit and rat origin. Exemplary PH20 polypeptides include those from human (SEQ ID NO:1), chimpanzee (SEQ ID NO:101), rhesus monkey (SEQ ID NO:102), cynomolgus monkey (SEQ ID NO:29), cow (e.g. SEQ ID NOS:11 and 64), mouse (SEQ ID NO:32), rat (SEQ ID NO:31), rabbit (SEQ ID NO:25), sheep (SEQ ID NOS:27, 63 and 65) and guinea pig (SEQ ID NO:30).

Reference to hyaluronan-degrading enzymes includes precursor hyaluronan-degrading enzyme polypeptides and mature hyaluronan-degrading enzyme polypeptides (such as those in which a signal sequence has been removed), truncated forms thereof that have activity, and includes allelic variants and species variants, variants encoded by splice variants, and other variants, including polypeptides that have at least 40%, 45%, 50%, 55%, 65%, 70%, 75%, 80%, 85%, 90%, 95%, 96%, 97%, 98%, 99% or more sequence identity to the precursor polypeptides set forth in SEQ ID NOS:1 and 10-48, 63-65, 67-102, or the mature forms thereof. For example, reference to hyaluronan-degrading enzyme also includes the human PH20 precursor polypeptide variants set forth in SEQ ID NOS:50-51. Hyaluronan-degrading enzymes also include those that contain chemical or posttranslational modifications and those that do not contain chemical or posttranslational modifications. Such modifications include, but are not limited to, PEGylation, albumination, glycosylation, farnesylation, carboxylation, hydroxylation, phosphorylation, and other polypeptide modifications known in the art. A truncated PH20 hyaluronidase is any C-terminal shortened form thereof, particularly forms that are truncated and neutral active when N-glycosylated.

As used herein, a “soluble PH20” refers to any form of PH20 that is soluble under physiologic conditions. A soluble PH20 can be identified, for example, by its partitioning into the aqueous phase of a Triton® X-114 solution at 37° C. (Bordier et al. (1981) J. Biol. Chem. 256:1604-1607). Membrane-anchored PH20, such as lipid-anchored PH20, including GPI-anchored PH20, will partition into the detergent-rich phase, but will partition into the detergent-poor or aqueous phase following treatment with Phospholipase-C. Included among soluble PH20 are membrane-anchored PH20 in which one or more regions associated with anchoring of the PH20 to the membrane have been removed or modified, where the soluble form retains hyaluronidase activity. Soluble PH20 also includes recombinant soluble PH20 and those contained in or purified from natural sources, such as, for example, testes extracts from sheep or cows. Exemplary of such soluble PH20 is soluble human PH20.

As used herein, soluble human PH20 or sHuPH20 includes PH20 polypeptides lacking all or a portion of the glycosylphosphatidylinositol (GPI) anchor sequence at the C-terminus such that upon expression, the polypeptides are soluble under physiological conditions. Solubility can be assessed by any suitable method that demonstrates solubility under physiologic conditions. Exemplary of such methods is the Triton® X-114 assay, that assesses partitioning into the aqueous phase and that is described above and in the examples. In addition, a soluble human PH20 polypeptide is, if produced in CHO cells, such as CHO-S cells, a polypeptide that is expressed and is secreted into the cell culture medium. Soluble human PH20 polypeptides, however, are not limited to those produced in CHO cells, but can be produced in any cell or by any method, including recombinant expression and polypeptide synthesis. Reference to secretion in CHO cells is definitional. Hence, if a polypeptide could be expressed and secreted in CHO cells and is soluble, i.e. partitions into the aqueous phase when extracted with Triton® X-114, it is a soluble PH20 polypeptide whether or not it is so-produced. The precursor polypeptides for sHuPH20 polypeptides can include a signal sequence, such as a heterologous or non-heterologous (i.e. native) signal sequence. Exemplary of the precursors are those that include a signal sequence, such as the native 35 amino acid signal sequence at amino acid positions 1-35 (see, e.g. amino acids 1-35 of SEQ ID NO:1).

As used herein, an “extended soluble PH20” or “esPH20” includes soluble PH20 polypeptides that contain residues up to the GPI anchor-attachment signal sequence and one or more contiguous residues from the GPI-anchor attachment signal sequence such that the esPH20 is soluble under physiological conditions. Solubility under physiological conditions can be determined by any method known to those of skill in the art. For example, it can be assessed by the Triton® X-114 assay described above and in the examples. In addition, as discussed above, a soluble PH20 is, if produced in CHO cells, such as CHO-S cells, a polypeptide that is expressed and is secreted into the cell culture medium. Soluble human PH20 polypeptides, however, are not limited to those produced in CHO cells, but can be produced in any cell or by any method, including recombinant expression and polypeptide synthesis. Reference to secretion in CHO cells is definitional. Hence, if a polypeptide could be expressed and secreted in CHO cells and is soluble, i.e. partitions into the aqueous phase when extracted with Triton® X-114, it is a soluble PH20 polypeptide whether or not it is so-produced. Human soluble esPH20 polypeptides include, in addition to residues 36-490, one or more contiguous amino acids from amino acid residue position 491 of SEQ ID NO:1, inclusive, such that the resulting polypeptide is soluble. Exemplary human esPH20 soluble polypeptides are those that have amino acids residues corresponding to amino acids 36-491, 36-492, 36-493, 36-494, 36-495, 36-496 and 36-497 of SEQ ID NO:1. Exemplary of these are those with an amino acid sequence set forth in any of SEQ ID NOS:151-154 and 185-187. Also included are allelic variants and other variants, such as any with 40%, 45%, 50%, 55%, 65%, 70%, 75%, 80%, 85%, 90%, 91%, 92%, 93%, 94%, 95%, 96%, 97%, 98%, 99% or greater sequence identity with the corresponding polypeptides of SEQ ID NOS:151-154 and 185-187 that retain neutral activity and are soluble. Reference to sequence identity refers to variants with amino acid substitutions.

As used herein, reference to “esPH20s” includes precursor esPH20 polypeptides and mature esPH20 polypeptides (such as those in which a signal sequence has been removed), truncated forms thereof that have enzymatic activity (retaining at least 1%, 10%, 20%, 30%, 40%, 50% or more of the full-length form) and are soluble, and includes allelic variants and species variants, variants encoded by splice variants, and other variants, including polypeptides that have at least 40%, 45%, 50%, 55%, 65%, 70%, 75%, 80%, 85%, 90%, 91%, 92%, 93%, 94%, 95%, 96%, 97%, 98%, 99% or more sequence identity to the precursor polypeptides set forth in SEQ ID NOS:1 and 3, or the mature forms thereof.

As used herein, reference to “esPH20s” also include those that contain chemical or posttranslational modifications and those that do not contain chemical or posttranslational modifications. Such modifications include, but are not limited to, PEGylation, albumination, glycosylation, farnesylation, carboxylation, hydroxylation, phosphorylation, and other polypeptide modifications known in the art.

As used herein, “soluble recombinant human PH20 (rHuPH20)” refers to a composition containing a soluble form of human PH20 as recombinantly expressed and secreted in Chinese Hamster Ovary (CHO) cells. Soluble rHuPH20 is encoded by a nucleic acid molecule that includes the signal sequence and is set forth in SEQ ID NO:49. The nucleic acid encoding soluble rHuPH20 is expressed in CHO cells which secrete the mature polypeptide. As produced in the culture medium, there is heterogeneity at the C-terminus so that the product includes a mixture of species that can include any one or more of SEQ ID NO:4 to SEQ ID NO:9 in various abundance.

Similarly, for other forms of PH20, such as the esPH20s, recombinantly expressed polypeptides and compositions thereof can include a plurality of species whose C-terminus exhibits heterogeneity. For example, compositions of recombinantly expressed esPH20 produced by expression of the polypeptide of SEQ ID NO:107, which encodes an esPH20 that has amino acids 36-497, can include forms with fewer amino acids, such as 36-496, 36-495.

As used herein, an “N-linked moiety” refers to an asparagine (N) amino acid residue of a polypeptide that is capable of being glycosylated by posttranslational modification of a polypeptide. Exemplary N-linked moieties of human PH20 include amino acids N82, N166, N235, N254, N368 and N393 of human PH20 set forth in SEQ ID NO:1.

As used herein, an “N-glycosylated polypeptide” refers to a PH20 polypeptide, or truncated form thereto, containing an oligosaccharide linkage of at least three N-linked amino acid residues, for example, N-linked moieties corresponding to amino acid residues N235, N368 and N393 of SEQ ID NO:1. An N-glycosylated polypeptide can include a polypeptide where three, four, five and up to all of the N-linked moieties are linked to an oligosaccharide. The N-linked oligosaccharides can include oligomannose, complex, hybrid or sulfated oligosaccharides, or other oligosaccharides and monosaccharides.

As used herein, an “N-partially glycosylated polypeptide” refers to a polypeptide that minimally contains an N-acetylglucosamine glycan linked to at least three N-linked moieties. A partially glycosylated polypeptide can include various glycan forms, including monosaccharides, oligosaccharides, and branched sugar forms, including those formed by treatment of a polypeptide with EndoH, EndoF1, EndoF2 and/or EndoF3.

As used herein, a “deglycosylated PH20 polypeptide” refers to a PH20 polypeptide in which fewer than all possible glycosylation sites are glycosylated. Deglycosylation can be affected, for example, by removing glycosylation, by preventing it, or by modifying the polypeptide to eliminate a glycosylation site. Particular N-glycosylation sites are not required for activity, whereas others are.

As used herein, a “conjugate” refers to a polypeptide linked directly or indirectly to one or more other polypeptides or chemical moieties. Such conjugates include fusion proteins, those produced by chemical conjugates and those produced by any other methods. For example, a conjugate refers to hyaluronan-degrading enzyme, such as a hyaluronidase or soluble PH20 polypeptide, linked directly or indirectly to one or more other polypeptides or chemical moieties, whereby at least one soluble PH20 polypeptide is linked, directly or indirectly to another polypeptide or chemical moiety, so long as the conjugate retains hyaluronidase activity.

As used herein, a “polymer” refers to any high molecular weight natural or synthetic moiety that is made up of repeating units. Polymers include, but are not limited to, polyethylene glycol moieities, dextran, cellulose and sialic acid. These and other exemplary polymers are described herein, and many are known in the art. For purposes herein, the polymer can be conjugated to, i.e. stably linked directly or indirectly via a linker, to a polypeptide. Such polymer conjugates typically increase serum half-life, and include, but are not limited to, sialic moieties, PEGylation moieties, dextran, and sugar and other moieties, such as for glycosylation. For example, hyaluronidases, such as a soluble PH20 or rHuPH20, can be conjugated to a polymer.

As used herein, “PEGylated” refers to covalent or other stable attachment of polymeric molecules, such as polyethylene glycol (PEGylation moiety PEG) to hyaluronan-degrading enzymes, such as hyaluronidases, typically to increase half-life of the hyaluronan-degrading enzyme.

As used herein, a “polymer-conjugated hyaluronan-degrading enzyme” refers to a hyaluronan-degrading enzyme that is linked directly or indirectly to a polymer. The linkage can be any type of linkage, including, but not limited to, ionic and covalent bonds, and any other sufficiently stable associated interaction. Reference to a polymer-conjugated hyaluronan-degrading enzyme means that the conjugate exhibits hyaluronidase activity. Typically, the polymer-conjugate exhibits at least 20%, 30%, 40%, 50%, 60%, 70%, 80%, 90% or more of the hyaluronidase activity compared to the hyaluronan-degrading enzyme that is not conjugated to a polymer.

As used herein, “activity” refers to a functional activity or activities of a polypeptide or portion thereof associated with a full-length (complete) protein. For example, active fragments of a polypeptide can exhibit an activity of a full-length protein. Functional activities include, but are not limited to, biological activity, catalytic or enzymatic activity, antigenicity (ability to bind or compete with a polypeptide for binding to an anti-polypeptide antibody), immunogenicity, ability to form multimers, and the ability to specifically bind to a receptor or ligand for the polypeptide.

As used herein, “hyaluronidase activity” refers to the ability to enzymatically catalyze the cleavage of hyaluronic acid. The United States Pharmacopeia (USP) XXII assay for hyaluronidase determines hyaluronidase activity indirectly by measuring the amount of higher molecular weight hyaluronic acid, or hyaluronan (HA), substrate remaining after the enzyme is allowed to react with the HA for 30 min at 37° C. (USP XXII-NF XVII (1990) 644-645 United States Pharmacopeia Convention, Inc, Rockville, Md.). A Reference Standard solution can be used in an assay to ascertain the relative activity, in units, of any hyaluronidase. In vitro assays to determine the hyaluronidase activity of hyaluronidases, such as PH20, including soluble PH20 and esPH20, are known in the art and described herein. Exemplary assays include the microturbidity assay that measures cleavage of hyaluronic acid by hyaluronidase indirectly by detecting the insoluble precipitate formed when the uncleaved hyaluronic acid binds with serum albumin and the biotinylated-hyaluronic acid assay that measures the cleavage of hyaluronic acid indirectly by detecting the remaining biotinylated-hyaluronic acid non-covalently bound to microtiter plate wells with a streptavidin-horseradish peroxidase conjugate and a chromogenic substrate. Reference Standards can be used, for example, to generate a standard curve to determine the activity in Units of the hyaluronidase being tested.

As used herein, specific activity refers to Units of activity per mg protein. The milligrams of hyaluronidase is defined by the absorption of a solution at 280 nm, assuming a molar extinction coefficient of approximately 1.7, in units of M⁻¹ cm⁻¹.

As used herein, “neutral active” refers to the ability of a PH20 polypeptide to enzymatically catalyze the cleavage of hyaluronic acid at neutral pH (e.g. at or about pH 7.0).

As used herein, a “GPI-anchor attachment signal sequence” is a C-terminal sequence of amino acids that directs addition of a preformed GPI-anchor to the polypeptide within the lumen of the ER. GPI-anchor attachment signal sequences are present in the precursor polypeptides of GPI-anchored polypeptides, such as GPI-anchored PH20 polypeptides. The C-terminal GPI-anchor attachment signal sequence typically contains a predominantly hydrophobic region of 8-20 amino acids, preceded by a hydrophilic spacer region of 8-12 amino acids, immediately downstream of the co-site, or site of GPI-anchor attachment. GPI-anchor attachment signal sequences can be identified using methods well known in the art. These include, but are not limited to, in silico methods and algorithms (see, e.g. Udenfriend et al. (1995) Methods Enzymol. 250:571-582; Eisenhaber et al. (1999) J. Biol. Chem. 292:741-758; Fankhauser et al. (2005) Bioinformatics 21:1846-1852; Omaetxebarria et al. (2007) Proteomics 7:1951-1960; and Pierleoni et al. (2008) BMC Bioinformatics 9:392), including those that are readily available on bioinformatic websites, such as the ExPASy Proteomics tools site (e.g. the WorldWideWeb site expasy.ch/tools/).

As used herein, “nucleic acids” include DNA, RNA and analogs thereof, including peptide nucleic acids (PNA) and mixtures thereof. Nucleic acids can be single- or double-stranded. When referring to probes or primers, which are optionally labeled, such as with a detectable label, such as a fluorescent or radiolabel, single-stranded molecules are contemplated. Such molecules are typically of a length such that their target is statistically unique or of low copy number (typically less than 5, generally less than 3) for probing or priming a library. Generally, a probe or primer contains at least 14, 16 or 30 contiguous nucleotides of sequence complementary to or identical to a gene of interest. Probes and primers can be 10, 20, 30, 50, 100 or more nucleic acids long.

As used herein, a peptide refers to a polypeptide that is greater than or equal to 2 amino acids in length, and less than or equal to 40 amino acids in length.

As used herein, the amino acids which occur in the various sequences of amino acids provided herein are identified according to their known, three-letter or one-letter abbreviations (Table 1). The nucleotides which occur in the various nucleic acid fragments are designated with the standard single-letter designations used routinely in the art.

As used herein, an “amino acid” is an organic compound containing an amino group and a carboxylic acid group. A polypeptide contains two or more amino acids. For purposes herein, amino acids include the twenty naturally-occurring amino acids, non-natural amino acids and amino acid analogs (i.e. amino acids wherein the α-carbon has a side chain).

As used herein, “amino acid residue” refers to an amino acid formed upon chemical digestion (hydrolysis) of a polypeptide at its peptide linkages. The amino acid residues described herein are presumed to be in the “L” isomeric form. Residues in the “D” isomeric form, which are so designated, can be substituted for any L-amino acid residue as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem. 243:3557-3559 (1968), and adopted in 37 C.F.R. §§1.821-1.822, abbreviations for amino acid residues are shown in Table 1:

TABLE 1
Table of Correspondence

| SYMBOL | | |
|---|---|---|
| 1-Letter | 3-Letter | AMINO ACID |
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | Proline |
| K | Lys | Lysine |
| H | His | Histidine |
| Q | Gln | Glutamine |
| E | Glu | Glutamic acid |
| Z | Glx | Glu and/or Gln |
| W | Trp | Tryptophan |
| R | Arg | Arginine |
| D | Asp | Aspartic acid |
| N | Asn | Asparagine |
| B | Asx | Asn and/or Asp |
| C | Cys | Cysteine |
| X | Xaa | Unknown or other |

All amino acid residue sequences represented herein by formulae have a left to right orientation in the conventional direction of amino-terminus to carboxyl-terminus. In addition, the phrase “amino acid residue” is defined to include the amino acids listed in the Table of Correspondence (Table 1) and modified and unusual amino acids, such as those referred to in 37 C.F.R. §§1.821-1.822, and incorporated herein by reference. Furthermore, it should be noted that a dash at the beginning or end of an amino acid residue sequence indicates a peptide bond to a further sequence of one or more amino acid residues, to an amino-terminal group such as NH₂ or to a carboxyl-terminal group such as COOH.

As used herein, the “naturally occurring α-amino acids” are the residues of those 20 α-amino acids found in nature which are incorporated into protein by the specific recognition of the charged tRNA molecule with its cognate mRNA codon in humans. Non-naturally occurring amino acids thus include, for example, amino acids or analogs of amino acids other than the 20 naturally-occurring amino acids and include, but are not limited to, the D-isostereomers of amino acids. Exemplary non-natural amino acids are described herein and are known to those of skill in the art.

As used herein, a DNA construct is a single- or double-stranded, linear or circular DNA molecule that contains segments of DNA combined and juxtaposed in a manner not found in nature. DNA constructs exist as a result of human manipulation, and include clones and other copies of manipulated molecules.

As used herein, a DNA segment is a portion of a larger DNA molecule having specified attributes. For example, a DNA segment encoding a specified polypeptide is a portion of a longer DNA molecule, such as a plasmid or plasmid fragment, which, when read from the 5′ to 3′ direction, encodes the sequence of amino acids of the specified polypeptide.

As used herein, the term polynucleotide means a single- or double-stranded polymer of deoxyribonucleotides or ribonucleotide bases read from the 5′ to the 3′ end. Polynucleotides include RNA and DNA, and can be isolated from natural sources, synthesized in vitro, or prepared from a combination of natural and synthetic molecules. The length of a polynucleotide molecule is given herein in terms of nucleotides (abbreviated “nt”) or base pairs (abbreviated “bp”). The term nucleotides is used for single- and double-stranded molecules where the context permits. When the term is applied to double-stranded molecules, it is used to denote overall length and will be understood to be equivalent to the term base pairs. It will be recognized by those skilled in the art that the two strands of a double-stranded polynucleotide can differ slightly in length and that the ends thereof can be staggered; thus, all nucleotides within a double-stranded polynucleotide molecule may not be paired. Such unpaired ends will, in general, not exceed 20 nucleotides in length.

As used herein, “similarity” between two proteins or nucleic acids refers to the relatedness between the sequence of amino acids of the proteins or the nucleotide sequences of the nucleic acids. Similarity can be based on the degree of identity and/or homology of sequences of residues and the residues contained therein. Methods for assessing the degree of similarity between proteins or nucleic acids are known to those of skill in the art. For example, in one method of assessing sequence similarity, two amino acid or nucleotide sequences are aligned in a manner that yields a maximal level of identity between the sequences. “Identity” refers to the extent to which the amino acid or nucleotide sequences are invariant. Alignment of amino acid sequences, and to some extent nucleotide sequences, also can take into account conservative differences and/or frequent substitutions in amino acids (or nucleotides). Conservative differences are those that preserve the physico-chemical properties of the residues involved. Alignments can be global (alignment of the compared sequences over the entire length of the sequences and including all residues) or local (alignment of a portion of the sequences that includes only the most similar region or regions).

“Identity” per se has an art-recognized meaning and can be calculated using published techniques. (See, e.g. Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exists a number of methods to measure identity between two polynucleotide or polypeptides, the term “identity” is well known to skilled artisans (Carillo and Lipton (1988) SIAM J. Appl. Math. 48:1073).

As used herein, “homologous” (with respect to nucleic acid and/or amino acid sequences) means about greater than or equal to 25% sequence homology, typically greater than or equal to 25%, 40%, 50%, 60%, 70%, 80%, 85%, 90% or 95% sequence homology; the precise percentage can be specified if necessary. For purposes herein, the terms “homology” and “identity” are often used interchangeably, unless otherwise indicated. In general, for determination of the percentage homology or identity, sequences are aligned so that the highest order match is obtained (see, e.g. Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje. G., Academic Press, 1987; and Sequence Analysis Primer, Gribskov, M. and Devereux, J., eds., M Stockton Press, New York, 1991; Carillo and Lipton (1988) SIAM J. Appl. Math. 48:1073). By sequence homology, the number of conserved amino acids is determined by standard alignment algorithm programs, and can be used with default gap penalties established by each supplier. Substantially homologous nucleic acid molecules would hybridize typically at moderate stringency or at high stringency all along the length of the nucleic acid of interest. Also contemplated are nucleic acid molecules that contain degenerate codons in place of codons in the hybridizing nucleic acid molecule.

Whether any two molecules have nucleotide sequences or amino acid sequences that are at least 60%, 70%, 80%, 85%, 90%, 95%, 96%, 97%, 98% or 99% “identical” or “homologous” can be determined using known computer algorithms such as the “FASTA” program, using for example, the default parameters as in Pearson et al. (1988) Proc. Natl. Acad. Sci. U.S.A. 85:2444 (other programs include the GCG program package (Devereux et al. (1984) Nucleic Acids Res. 12(1):387), BLASTP, BLASTN, FASTA (Altschul et al. (1990) J. Mol. Biol. 215:403-410; Guide to Huge Computers, Martin J. Bishop, ed., Academic Press, San Diego, 1994; and Carillo and Lipton (1988) SIAM. J. Appl. Math. 48:1073). For example, the BLAST function of the National Center for Biotechnology Information database can be used to determine identity. Other commercially or publicly available programs include DNAStar “MegAlign” program (Madison, Wis.) and the University of Wisconsin Genetics Computer Group (UWG) “Gap” program (Madison Wis.). Percent homology or identity of proteins and/or nucleic acid molecules can be determined, for example, by comparing sequence information using a GAP computer program (e.g. Needleman et al. (1970) J. Mol. Biol. 48:443, as revised by Smith and Waterman (Adv. Appl. Math. 2:482 (1981)). Briefly, the GAP program defines similarity as the number of aligned symbols (i.e. nucleotides or amino acids) which are similar, divided by the total number of symbols in the shorter of the two sequences. Default parameters for the GAP program can include: (1) a unary comparison matrix (containing a value of 1 for identities and 0 for non-identities) and the weighted comparison matrix of Gribskov et al. (1986) Nucleic Acids Res. 14:6745, as described by Schwartz and Dayhoff, eds., in “Atlas of Protein Sequence and Structure,” National Biomedical Research Foundation, pp. 353-358 (1979); (2) a penalty of 3.0 for each gap and an additional 0.10 penalty for each symbol in each gap; and (3) no penalty for end gaps.

Therefore, as used herein, the term “identity” or “homology” represents a comparison between a test and a reference polypeptide or polynucleotide. As used herein, the term at least “90% identical to” refers to percent identities from 90 to 99.99 relative to the reference nucleic acid or amino acid sequence of the polypeptide. Identity at a level of 90% or more is indicative of the fact that, assuming for exemplification purposes, a test and reference polypeptide length of 100 amino acids are compared. No more than 10% (i.e. 10 out of 100) of the amino acids in the test polypeptide differ from that of the reference polypeptide. Similar comparisons can be made between test and reference polynucleotides. Such differences can be represented as point mutations randomly distributed over the entire length of a polypeptide or they can be clustered in one or more locations of varying length up to the maximum allowable, e.g. 10/100 amino acid difference (approximately 90% identity). Differences are defined as nucleic acid or amino acid substitutions, insertions or deletions. At the level of homologies or identities above about 85-90%, the result should be independent of the program and gap parameters set; such high levels of identity can be assessed readily, often by manual alignment without relying on software.

As used herein, an aligned sequence refers to the use of homology (similarity and/or identity) to align corresponding positions in a sequence of nucleotides or amino acids. Typically, two or more sequences that are related by 50% or more identity are aligned. An aligned set of sequences refers to 2 or more sequences that are aligned at corresponding positions and can include aligning sequences derived from RNAs, such as ESTs and other cDNAs, aligned with genomic DNA sequence.

As used herein, “primer” refers to a nucleic acid molecule that can act as a point of initiation of template-directed DNA synthesis under appropriate conditions (e.g. in the presence of four different nucleoside triphosphates and a polymerization agent, such as DNA polymerase, RNA polymerase or reverse transcriptase) in an appropriate buffer and at a suitable temperature. It will be appreciated that certain nucleic acid molecules can serve as a “probe” and as a “primer.” A primer, however, has a 3′ hydroxyl group for extension. A primer can be used in a variety of methods, including, for example, polymerase chain reaction (PCR), reverse-transcriptase (RT)-PCR, RNA PCR, LCR, multiplex PCR, panhandle PCR, capture PCR, expression PCR, 3′ and 5′ RACE, in situ PCR, ligation-mediated PCR and other amplification protocols.

As used herein, “primer pair” refers to a set of primers that includes a 5′ (upstream) primer that hybridizes with the 5′ end of a sequence to be amplified (e.g. by PCR) and a 3′ (downstream) primer that hybridizes with the complement of the 3′ end of the sequence to be amplified.

As used herein, “specifically hybridizes” refers to annealing, by complementary base-pairing, of a nucleic acid molecule (e.g. an oligonucleotide) to a target nucleic acid molecule. Those of skill in the art are familiar with in vitro and in vivo parameters that affect specific hybridization, such as length and composition of the particular molecule. Parameters particularly relevant to in vitro hybridization further include annealing and washing temperature, buffer composition and salt concentration. Exemplary washing conditions for removing non-specifically bound nucleic acid molecules at high stringency are 0.1×SSPE, 0.1% SDS, 65° C., and at medium stringency are 0.2×SSPE, 0.1% SDS, 50° C. Equivalent stringency conditions are known in the art. The skilled person can readily adjust these parameters to achieve specific hybridization of a nucleic acid molecule to a target nucleic acid molecule appropriate for a particular application. Complementary, when referring to two nucleotide sequences, means that the two sequences of nucleotides are capable of hybridizing, typically with less than 25%, 15% or 5% mismatches between opposed nucleotides. If necessary, the percentage of complementarity will be specified. Typically the two molecules are selected such that they will hybridize under conditions of high stringency.

As used herein, substantially identical to a product means sufficiently similar so that the property of interest is sufficiently unchanged so that the substantially identical product can be used in place of the product.

As used herein, it also is understood that the terms “substantially identical” or “similar” varies with the context as understood by those skilled in the relevant art.

As used herein, an “allelic variant” or “allelic variation” references any of two or more alternative forms of a gene occupying the same chromosomal locus. Allelic variation arises naturally through mutation, and can result in phenotypic polymorphism within populations. Gene mutations can be silent (no change in the encoded polypeptide) or can encode polypeptides having altered amino acid sequence. The term “allelic variant” also is used herein to denote a protein encoded by an allelic variant of a gene. Typically the reference form of the gene encodes a wildtype form and/or predominant form of a polypeptide from a population or single reference member of a species. Typically, allelic variants, which include variants between and among species typically have at least 80%, 90% or greater amino acid identity with a wildtype and/or predominant form from the same species; the degree of identity depends upon the gene and whether comparison is interspecies or intraspecies. Generally, intraspecies allelic variants have at least about 80%, 85%, 90% or 95% identity or greater with a wildtype and/or predominant form, including 96%, 97%, 98%, 99% or greater identity with a wildtype and/or predominant form of a polypeptide. Reference to an allelic variant herein generally refers to variations in proteins among members of the same species.

As used herein, “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for that gene or allele. When a subject has two different alleles of a gene, the subject is said to be heterozygous for the gene. Alleles of a specific gene can differ from each other in a single nucleotide or several nucleotides, and can include modifications such as substitutions, deletions and insertions of nucleotides. An allele of a gene also can be a form of a gene containing a mutation.

As used herein, species variants refer to variants in polypeptides among different species, including different mammalian species, such as mouse and human.

For example, for PH20, exemplary of species variants provided herein are primate PH20, such as, but not limited to, human, chimpanzee, macaque and cynomolgus monkey. Generally, species variants have 70%, 75%, 80%, 85%, 90%, 91%, 92%, 93%, 94%, 95%, 96%, 97%, 98% or sequence identity. Corresponding residues between and among species variants can be determined by comparing and aligning sequences to maximize the number of matching nucleotides or residues, for example, such that identity between the sequences is equal to or greater than 95%, equal to or greater than 96%, equal to or greater than 97%, equal to or greater than 98% or equal to greater than 99%. The position of interest is then given the number assigned in the reference nucleic acid molecule. Alignment can be effected manually or by eye, particularly, where sequence identity is greater than 80%.

As used herein, a human protein is one encoded by a nucleic acid molecule, such as DNA, present in the genome of a human, including all allelic variants and conservative variations thereof. A variant or modification of a protein is a human protein if the modification is based on the wildtype or prominent sequence of a human protein.

As used herein, a splice variant refers to a variant produced by differential processing of a primary transcript of genomic DNA that results in more than one type of mRNA.

As used herein, modification is in reference to modification of a sequence of amino acids of a polypeptide or a sequence of nucleotides in a nucleic acid molecule and includes deletions, insertions, and replacements (e.g. substitutions) of amino acids and nucleotides, respectively. Exemplary of modifications are amino acid substitutions. An amino-acid substituted polypeptide can exhibit 65%, 70%, 80%, 85%, 90%, 91%, 92%, 93%, 94%, 95%, 96%, 97%, 98% or more sequence identity to a polypeptide not containing the amino acid substitutions. Amino acid substitutions can be conservative or non-conservative. Generally, any modification to a polypeptide retains an activity of the polypeptide. Methods of modifying a polypeptide are routine to those of skill in the art, such as by using recombinant DNA methodologies.

As used herein, suitable conservative substitutions of amino acids are known to those of skill in the art and can be made generally without altering the biological activity of the resulting molecule. Those of skill in the art recognize that, in general, single amino acid substitutions in non-essential regions of a polypeptide do not substantially alter biological activity (see, e.g. Watson et al. in “Molecular Biology of the Gene,” 4th Edition, 1987, The Benjamin/Cummings Pub. co., p. 224). Such substitutions can be made in accordance with those set forth in Table 2 as follows:

TABLE 2

| | Exemplary |
|---|---|
| Original residue | conservative substitution |
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with known conservative substitutions.

As used herein, the term “promoter” means a portion of a gene containing DNA sequences that provide for the binding of RNA polymerase and initiation of transcription. Promoter sequences are commonly, but not always, found in the 5′ non-coding region of genes.

As used herein, an isolated or purified polypeptide or protein or biologically-active portion thereof is substantially free of cellular material or other contaminating proteins from the cell or tissue from which the protein is derived, or substantially free from chemical precursors or other chemicals when chemically synthesized. Preparations can be determined to be substantially free if they appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound, however, can be a mixture of stereoisomers. In such instances, further purification might increase the specific activity of the compound.

Hence, reference to a substantially purified polypeptide, such as a substantially purified soluble PH20, refers to preparations of proteins that are substantially free of cellular material and includes preparations of proteins in which the protein is separated from cellular components of the cells from which it is isolated or recombinantly-produced. In one embodiment, the term “substantially free of cellular material” includes preparations of enzyme proteins having less that about 30% (by dry weight) of non-enzyme proteins (also referred to herein as a contaminating protein), generally less than about 20% of non-enzyme proteins or 10% of non-enzyme proteins or less that about 5% of non-enzyme proteins. When the enzyme protein is recombinantly produced, it also is substantially free of culture medium, i.e. culture medium represents less than about or at 20%, 10% or 5% of the volume of the enzyme protein preparation.

As used herein, the term substantially free of chemical precursors or other chemicals includes preparations of enzyme proteins in which the protein is separated from chemical precursors or other chemicals that are involved in the synthesis of the protein. The term includes preparations of enzyme proteins having less than about 30% (by dry weight), 20%, 10%, 5% or less of chemical precursors or non-enzyme chemicals or components.

As used herein, synthetic, with reference to, for example, a synthetic nucleic acid molecule or a synthetic gene or a synthetic peptide refers to a nucleic acid molecule or polypeptide molecule that is produced by recombinant methods and/or by chemical synthesis methods.

As used herein, production by recombinant means or using recombinant DNA methods means the use of the well-known methods of molecular biology for expressing proteins encoded by cloned DNA.

As used herein, vector (or plasmid) refers to discrete elements that are used to introduce a heterologous nucleic acid into cells for either expression or replication thereof. The vectors typically remain episomal, but can be designed to effect integration of a gene or portion thereof into a chromosome of the genome. Also contemplated are vectors that are artificial chromosomes, such as yeast artificial chromosomes and mammalian artificial chromosomes. Selection and use of such vehicles are well known to those of skill in the art.

As used herein, an expression vector includes vectors capable of expressing DNA that is operatively linked with regulatory sequences, such as promoter regions, that are capable of effecting expression of such DNA fragments. Such additional segments can include promoter and terminator sequences, and optionally can include one or more origins of replication, one or more selectable markers, an enhancer, or a polyadenylation signal. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, vector also includes “virus vectors” or “viral vectors.” Viral vectors are engineered viruses that are operatively linked to exogenous genes to transfer (as vehicles or shuttles) the exogenous genes into cells.

As used herein, “operably” or “operatively linked” when referring to DNA segments means that the segments are arranged so that they function in concert for their intended purposes, e.g. transcription initiates downstream of the promoter and upstream of any transcribed sequences. The promoter is usually the domain to which the transcriptional machinery binds to initiate transcription and proceeds through the coding segment to the terminator.

As used herein the term “assessing” is intended to include quantitative and qualitative determination in the sense of obtaining an absolute value for the activity of a protein, such as an enzyme, or a domain thereof, present in the sample, and also of obtaining an index, ratio, percentage, visual or other value indicative of the level of the activity. Assessment can be direct or indirect. For example, the chemical species actually detected need not of course be the enzymatically cleaved product itself but can for example be a derivative thereof or some further substance. For example, detection of a cleavage product can be a detectable moiety such as a fluorescent moiety.

As used herein, biological activity refers to the in vivo activities of a compound or physiological responses that result upon in vivo administration of a compound, composition or other mixture. Biological activity, thus, encompasses therapeutic effects and pharmaceutical activity of such compounds, compositions and mixtures. Biological activities can be observed in in vitro systems designed to test or use such activities. Thus, for purposes herein a biological activity of a hyaluronidase enzyme is its degradation of hyaluronic acid.

As used herein, equivalent, when referring to two sequences of nucleic acids, means that the two sequences in question encode the same sequence of amino acids or equivalent proteins. When equivalent is used in referring to two proteins or peptides, it means that the two proteins or peptides have substantially the same amino acid sequence with only amino acid substitutions that do not substantially alter the activity or function of the protein or peptide. When equivalent refers to a property, the property does not need to be present to the same extent (e.g. two peptides can exhibit different rates of the same type of enzymatic activity), but the activities are usually substantially the same.

As used herein, “modulate” and “modulation” or “alter” refer to a change of an activity of a molecule, such as a protein. Exemplary activities include, but are not limited to, biological activities, such as signal transduction. Modulation can include an increase in the activity (i.e. up-regulation or agonist activity), a decrease in activity (i.e. down-regulation or inhibition) or any other alteration in an activity (such as a change in periodicity, frequency, duration, kinetics or other parameter). Modulation can be context dependent and typically, modulation is compared to a designated state, for example, the wildtype protein, the protein in a constitutive state, or the protein as expressed in a designated cell type or condition.

As used herein, direct administration refers to a composition that is administered without dilution.

As used herein, a single dosage formulation refers to a formulation for use only once. Typically, a single dosage formulation is for direct administration.

As used herein, a multiple dosage formulation refers to a formulation for use in repeat administrations.

As used herein, a composition refers to any mixture. It can be a solution, suspension, liquid, powder, paste, aqueous, non-aqueous or any combination thereof.

As used herein, a combination refers to any association between or among two or more items. The combination can be two or more separate items, such as two compositions or two collections, can be a mixture thereof, such as a single mixture of the two or more items, or any variation thereof. The elements of a combination are generally functionally associated or related.

As used herein, “intravenous administration” refers to delivery of a therapeutic directly into a vein.

As used herein, “treating” a subject with a disease or condition means that the subject's symptoms are partially or totally alleviated, or remain static following treatment. Hence, treatment encompasses prophylaxis, therapy and/or cure. Prophylaxis refers to prevention of a potential disease and/or a prevention of worsening of symptoms or progression of a disease.

As used herein, a pharmaceutically effective agent includes any therapeutic agent or bioactive agent, including, but not limited to, for example, chemotherapeutics, anesthetics, vasoconstrictors, dispersing agents, conventional therapeutic drugs, including small molecule drugs and therapeutic proteins.

As used herein, treatment means any manner in which the symptoms of a condition, disorder or disease or other indication, are ameliorated or otherwise beneficially altered.

As used herein, therapeutic effect means an effect resulting from treatment of a subject that alters, typically improves or ameliorates the symptoms of a disease or condition or that cures a disease or condition. A therapeutically effective amount refers to the amount of a composition, molecule or compound which results in a therapeutic effect following administration to a subject.

As used herein, the term “subject” refers to an animal, including a mammal, such as a human being.

As used herein, a patient refers to a human subject exhibiting symptoms of a disease or disorder.

As used herein, “about the same” means within an amount that one of skill in the art would consider to be the same or to be within an acceptable range of error. For example, typically, for pharmaceutical compositions, within at least 1%, 2%, 3%, 4%, 5% or 10% is considered about the same. Such amount can vary depending upon the tolerance for variation in the particular composition by subjects.

As used herein, dosing regime refers to the amount of agent, for example, the composition containing an hyalruonan-degrading enzyme, for example a soluble hyaluronidase or other agent, administered, and the frequency of administration. The dosing regime is a function of the disease or condition to be treated, and thus can vary.

As used herein, frequency of administration refers to the time or interval between successive administrations of treatment. For example, frequency can be days, weeks or months. For example, frequency can be more than once weekly, for example, twice a week, three times a week, four times a week, five times a week, six times a week or daily. Frequency also can be one, two, three or four weeks. The particular frequency is a function of the particular disease or condition treated. Generally, frequency is more than once weekly, and generally is twice weekly.

As used herein, a “cycle of administration” refers to the repeated schedule of the dosing regime of administration of the agent, such as a hyaluronan-degrading enzyme, that is repeated over successive administrations. For example, an exemplary cycle of administration is a 28 day cycle with administration twice weekly for three weeks, followed by one-week of discontinued dosing.

As used herein, when referencing dosage based on mg/kg of the subject, an average human subject is considered to have a mass of about 70 kg-75 kg, such as 70 kg and a body surface area (BSA) of 1.73.

As used herein, a “therapeutically effective amount” or a “therapeutically effective dose” refers to the quantity of an agent, compound, material, or composition containing a compound that is at least sufficient to produce a therapeutic effect. Hence, it is the quantity necessary for preventing, curing, ameliorating, arresting or partially arresting a symptom of a disease or disorder.

As used herein, a corticosteroid refers to a group of hormones that are naturally, synthetically or recombinantly generated, and that have an activity of a natural hormone that is secreted by the adrenal cortex. Corticosteroids can influence biological process, including, but not limited to, carbohydrate and protein metabolism, maintenance of serum glucose levels, electrolyte and water balance, and functions of the cardiovascular system, the skeletal muscle, the kidneys, and other organs. Corticosteroids include glucocorticoids, mineralcorticoids and corticotropins.

As used herein, a glucocorticoid is a corticosteroid that exhibits anti-inflammatory activity, and that also can affect glucose utilization, fat metabolism and bone development. Exemplary hormones that exhibit a glucocorticoid activity include, but are not limited to cortisone, hydrocortisone, dexamethasone, prednisone, prednisolone, betamethasone and triamcinolone.

As used herein, “adverse effect” or “side effect” refers to a harmful, deleterious and/or undesired effect of administering an agent, such as a polymer- (e.g. PEG) conjugated hyaluronan-degrading enzyme, such as PEGPH20, to a subject. Exemplary of side effects are musculoskeletal side effects. Side effects or adverse effects are graded on toxicity and various toxicity scales exist providing definitions for each grade. Exemplary of such scales are toxicity scales of the National Cancer Institute Common Toxicity Criteria version 2.0, the World Health Organization or Common Terminology Criteria for Adverse Events (CTCAE) scale. Generally, the scale is as follows: Grade 1=mild side effects; Grade 2=moderate side effects; Grade 3=Severe side effects; Grade 4=Life Threatening or Disabling side-effects; Grade 5=Fatal. Assigning grades of severity is within the experience of a physician or other health care professional.

As used herein, “musculoskeletal effect” or “musculoskeletal side effect” refers to effects on the system of muscles, tendons, ligaments, bones, joints and associated tissues. Musculoskeletal side effects include muscle and joint pain, stiffness of upper and lower extremities, cramping, myositis, muscle soreness and tenderness over the entire body, weakness, fatigue and a decrease in range of motion at knee and elbow joints. It is within the level of a skilled physician to assign grades of severity of observed or measured musculoskeletal side effects based on toxicity scales. It also is within the level of a skilled physician to assign a dose limiting toxicity (DLT) to an observed musculoskeletal side effect.

As used herein, “ameliorating,” “preventing,” or “reducing” a side effect or adverse event, or variations thereof, refers to lessening adverse effects or side effects, whether permanent or temporary, lasting or transient. For purposes herein, a side effect of an administered hyaluronan-degrading enzyme, such as a musculoskeletal side effect, is deemed ameliorated by a corticosteroid when there is a reduction or lessening in the grade of severity measured on a toxicity scale for the side effect in the presence of the corticosteroid compared to in its absence. In one example, a side effect is ameliorated when the observed or measured toxicity of an administered anti-hyaluronan agent (observed following single dosage administration, multiple dosage administration or by virtue of the dosage regime) of Grade 3 or higher is reduced to a Grade 1 or Grade 2 in the presence of a corticosteroid. In another example, a side effect is ameliorated when the DLT of an administered anti-hyaluronan agent is eliminated or increased following administration of the corticosteroid. For example, a side effect is ameliorated when a DLT of 0.05 mg/mL from an administered anti-hyaluronan agent is eliminated or increased by administration of a corticosteroid to the subject, such that the same dose or a higher dose of an anti-hyaluronan agent can be administered with reduced side effect or when the DLT is increased to greater than 0.05 mg/mL, such as 0.5 mg/mL.

As used herein, unit dose form refers to physically discrete units suitable for human and animal subjects and packaged individually as is known in the art.

As used herein, a single dosage formulation refers to a formulation as a single dose.

As used herein, formulation for direct administration means that the composition does not require further dilution for administration.

As used herein, an “article of manufacture” is a product that is made and sold. As used throughout this application, the term is intended to encompass anti-hyaluronan agents, for example hyaluronan-degrading enzyme, such as hyaluronidase, and second agent compositions contained in articles of packaging. For example, a second agent is a corticosteroid.

As used herein, fluid refers to any composition that can flow. Fluids thus encompass compositions that are in the form of semi-solids, pastes, solutions, aqueous mixtures, gels, lotions, creams and other such compositions.

As used herein, a combination, such as a combination of compositions provided herein, refers to an association of elements of the combination.

As used herein, a kit refers to a combination of components, such as a combination of the compositions herein and another item for a purpose including, but not limited to, reconstitution, activation, and instruments/devices for delivery, administration, diagnosis, and assessment of a biological activity or property. Kits optionally include instructions for use.

As used herein, a cellular extract or lysate refers to a preparation or fraction which is made from a lysed or disrupted cell.

As used herein, animal includes any animal, such as, but not limited to primates, including humans, gorillas and monkeys; rodents, such as mice and rats; fowl, such as chickens; ruminants, such as goats, cows, deer and sheep; pigs; and other animals. Non-human animals exclude humans as the contemplated animal. The hyaluronidases provided herein are from any source, including animal, plant, prokaryotic and fungal. Most hyaluronidases are of animal origin, including mammalian origin. Generally, hyaluronidases are of human origin.

As used herein, a control refers to a sample that is substantially identical to the test sample, except that it is not treated with a test parameter, or, if it is a plasma sample, it can be from a normal volunteer not affected with the condition of interest. A control also can be an internal control.

As used herein, the singular forms “a,” “an” and “the” include plural referents unless the context clearly dictates otherwise. Thus, for example, reference to a compound comprising or containing “an extracellular domain” includes compounds with one or a plurality of extracellular domains.

As used herein, ranges and amounts can be expressed as “about” a particular value or range. About also includes the exact amount. Hence “about 5 bases” means “about 5 bases” and also “5 bases.”

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. 11:1726 (1972)).

### B. METHODS FOR TREATING INSULIN RESISTANCE

Provided herein are methods of treating, ameliorating or reversing insulin resistance. In particular, the methods provided herein include methods of administering a polymer-conjugated hyaluronan-degrading enzyme, for example a hyaluronidase, such as PEGPH20, for use in the treatment, amelioration or reversal of insulin resistance. The methods provided herein can be used to reduce the hyaluronic acid (HA) content in the extracellular matrix (ECM) of insulin resistant tissue, for example in the ECM of muscle. The methods provided herein can be used in the treatment or prevention of any disease or condition associated with insulin resistance, such as diabetes or cardiovascular disease, by reducing, preventing or reversing the onset of disease.

1. Normal Glucose/Insulin Action

A continuous supply of glucose is necessary to ensure proper function and survival of all organs. In response to an elevation in plasma glucose and amino acids, for example, after consumption of a meal, insulin is released from the β cells of the islets of Langerhans in the pancreas. In an effort to maintain an ambient plasma glucose concentration of between ˜80 and 140 mg/dL, the pancreatic β cells will secrete whatever amount of insulin is required to maintain this concentration. As blood glucose levels are lowered, insulin output is reduced, with the result that blood glucose is maintained at approximately 5 mmol/L (90 mg/dL).

In individuals with normal metabolism, insulin stimulates glucose uptake, utilization, and storage in insulin sensitive tissues, while suppressing hepatic (liver) glucose production and release. Once secreted, insulin stimulates the transport of glucose into muscle and fat cells by increasing the concentration of the glucose transporter isoform Glut4 at the cell surface. This results in a 10- to 40-fold increase in cellular glucose uptake. Thus, the action of insulin to lower blood glucose levels results from suppression of hepatic glucose production and increased glucose uptake into muscle and fat. Insulin is also involved in the inhibition of lipolysis, the breakdown of lipids involving hydrolysis of triglycerides into free fatty acids (FFA), in adipocytes.

The primary targets for insulin are skeletal and cardiac muscle, adipose (fat) tissue, and the liver. Muscle has long been considered the major site of insulin-stimulated glucose uptake in vivo, with adipose tissue contributing relatively little to total body glucose disposal. For example, skeletal muscle plays an integral role in regulating whole-body homeostasis, with approximately 70-80% of ingested glucose taken up by skeletal muscle and either stored as glycogen or oxidized for energy. Skeletal muscle oxidative capacity can be a key predictor of whole-body insulin action.

2. Insulin Resistance

Insulin resistance is a physiological condition characterized by an inadequate response by insulin-sensitive tissues, such as the liver, skeletal muscle and adipose tissue, to normal circulating concentrations of insulin, resulting in increased blood glucose levels (i.e. hyperglycemia). In individuals with insulin resistance, insulin becomes less effective at lowering blood sugars by failing to inhibit hepatic glucose production, lipolysis, and skeletal muscle glucose uptake, leading to relative hyperglycemia and increased plasma levels of FFA.

A connection exists between obesity, reduced skeletal muscle fatty acid oxidation, and insulin resistance. Insulin-stimulated glucose transport is markedly decreased in the skeletal muscle of obese individuals and in patients with type 2 diabetes. Since skeletal muscle is the primary site of glucose uptake in the human body, its inability to utilize glucose in response to insulin could explain the reduced disposal of glucose observed in insulin-resistant individuals.

There are a number of contributing factors responsible for insulin resistance. Insulin resistance can result from a combination of genetic and environmental factors, and is a common condition associated with obesity, aging, and a sedentary lifestyle. For example, obesity is regarded as a pathological condition with abnormal fat accumulation as a result of relatively excessive calorie intake due to genetic and environmental conditions, and is a target or medical treatment. The occurrence of obesity and insulin resistance in the general population has resulted in it being considered to be an epidemic and a huge health problem. Insulin resistant individuals typically have elevated FFA concentrations compared to healthy individuals. Elevated FFA levels are not directly responsible for insulin resistance, but in obesity-induced insulin resistance, skeletal muscle is faced with increasing amounts of lipid which it is unable to oxidize. As a consequence, lipids accumulate within the muscle cell, and play a role in the development of insulin resistance. Insulin resistance also is associated with defects in the vasculature of tissues, including skeletal muscle. To be effective, insulin must enter the bloodstream from pancreatic β-cells where it circulates to various tissues and cells and exits the bloodstream to reach target tissues to mediate its effects (Richards et al. (2010) Endocrine Rev. 31:343-363). Insulin normally stimulates vasodilation, thereby effecting capillary or microvascular recruitment, in order to increase nutritive blood flow and promote transport of insulin and glucose to the muscle interstitium (Richards et al. (2010) Endocrine Rev. 31:343-363). Tissues from obese/insulin-resistant subjects, including skeletal muscle, generally have fewer capillaries and more large vessels as compared with lean subjects. For example, insulin resistance is associated with a decreased blood flow, which can be due to decreased capillarization in skeletal muscle (Lind et al. (1993) Am. Heart. J. 125:1494-1497). The effect of decreased tissue vascularization, and in particular muscle vascularization, reduces the ability of insulin and other metabolites and nutrients to reach tissue cells where they can be utilized. Indeed, it has been demonstrated that the movement of insulin within the muscle interstitium is impaired in insulin resistance (Richards et al. (2010) Endocrine Rev. 31:343-363).

In response to the relative hyperglycemia, there is a compensatory response by the pancreatic β cells, which secrete more insulin. The more resistant an individual is to insulin-mediated glucose disposal, the greater will be the degree of compensatory hyperinsulinemia, a condition in which there are excess levels of insulin circulating in the blood than required relative to the level of glucose. The excess insulin increases skeletal muscle glucose uptake and inhibits hepatic glucose production in order to maintain normal blood glucose levels (normoglycemia). Thus, insulin resistant individuals maintain normoglycemia through overproduction and secretion of insulin. Long-term insulin resistance and overproduction and secretion of insulin eventually leads to pancreatic β cell failure, resulting initially in pre-diabetes and glucose intolerance and later progressing to hyperglycemia and type 2 diabetes. Besides being a fundamental aspect of the etiology of type 2 diabetes, insulin resistance is also an early marker for a number of diseases and conditions including hypertension, dyslipidemia, fatty liver, atherosclerosis, cardiovascular disease, the metabolic syndrome, and polycystic ovarian disease.

3. Hyaluronan and Insulin Resistance

It is found herein that hyaluronan (HA) is increased in the ECM of insulin resistant tissue, in particular in skeletal muscle, and is associated with insulin resistance. The extracellular matrix (ECM) is a complex structural entity surrounding and supporting the cells in tissues. The ECM is constituted by diverse composite structures, which determine the physical characteristics of tissues and many of the biological properties of cells embedded in it. The ECM not only provides strength and physical support for tissues and organisms, but also exerts profound influences on the embedded cells, playing an essential role in organogenesis, growth, function, and many human diseases. The ECM is a reservoir for a wide range of biomolecules such as growth factors, enzymes, cytokines and hormones. In addition, the ECM communicates directly or indirectly with the intracellular signaling pathways that direct cells to carry out specific functions. The ECM also provides a mesh through which cells and other macromolecules can move.

The major components of the ECM are fibrous proteins that provide tensile strength (e.g. various collagens and elastin), multi-adhesive matrix proteins (e.g. fibronectin, laminin, and tenascin) that attach cells to diverse components of the ECM, and proteoglycans (PGs) that are largely responsible for the volume of the ECM. Different combinations of these components tailor the strength of the ECM for different purposes.

Hyaluronan (or hyaluronic acid, hyaluronate, HA), is a large, negatively charged, unbranched polymer. It is a non-sulfated glycosaminoglycan composed of repeating polymeric disaccharides of D-glucuronic acid and N-acetyl-D-glucosamine linked by a glucuronidic β(1,3) bond ([-β(1,4)-GlcUA-β(1,3)-GlcNAc-]ₙ). In normal physiological conditions, HA is made up of 2,000-25,000 disaccharides, corresponding to polysaccharides with relative molecular masses of 10⁶-10⁷ and polymer lengths of 2-25 μm. Although HA belongs to the family of glycosaminoglycans, it differs from other glycosaminoglycans in many ways. Other glycosaminoglycans are made as proteoglycans that are synthesized and assembled in the rough endoplasmic reticulum and Golgi apparatus, and are secreted in a similar way to other glycoproteins. HA, however, is synthesized as an unmodified polysaccharide and extruded through the plasma membrane onto the cell surface or into the ECM while it is being synthesized.

HA is widely distributed and abundant in the ECM, especially in skeletal tissue. After synthesis and extrusion, HA forms a highly viscous gel, creating a barrier to bulk fluid flow through the interstitial collagenous matrix due to its viscosity and ability to retain water, thus making the ECM resilient to compression. HA has multiple functions, such as space filling, hydration, lubrication of joints, and providing a matrix through which cells can migrate. Due to HA's ability to regulate the porosity and malleability of the ECM and its interactions with proteoglycans and other extracellular macromolecules that are important in the assembly of extracellular and pericellular matrices, HA is an important contributor and regulator of ECM remodeling.

HA also is an ECM component that normally regulates vascular integrity (Lennon et al. (2011) Am. J. Cardiovasc. Dis. 1:200-213). The predominant form of HA associated with the vasculature in vivo is a high molecular weight HA that associates with other ECM components, including proteoglycans and other glycosaminoglycans and other serum proteins, to make up endothelial glycocalyx located on the luminal side of the endothelium of blood vessels. The endothelial glycocalyx exhibits vasculoprotective properties such as regulating vascular permeability. The HA is aqueous and exhibits viscous and elastic properties that contribute to the filtering function of the endothelial glycocalyx.

It is found herein that HA is increased in insulin resistant skeletal muscle. Due to the role of HA in many cellular processes, including in vascular integrity, it is believed that its aberrant accumulation in skeletal muscle is associated with the development and progression of insulin resistance and associated diseases and conditions characterized by insulin resistance. For example, the role of accumulated or increased HA to decreased vascularization is confirmed by studies herein that show that removing HA using a hyaluronan-degrading enzyme results in increased muscle vascularization as assessed by the vascular marker CD31. This demonstrates that increased HA in skeletal muscle likely acts as a physical barrier and as a persistent factor in skeletal muscle insulin resistance due to decreased muscle vascularization and decreased accessibility of glucose and insulin.

4. Targeting HA by Hyaluronan-Degrading Enzyme to Reverse Insulin Resistance

Based on the findings herein that HA is increased in insulin resistant skeletal muscle and is associated with insulin resistance, a method for reversing or preventing insulin resistance by targeting removal of HA is provided herein. Hyaluronan-degrading enzymes, such as hyaluronidases, are a family of enzymes that degrade HA by cleaving the hyaluronan disaccharide polymers. Hyaluronan-degrading enzymes generally include enzymes that cleave the β-1→4 glycosidic bond in the hyaluronan chain or polymer. Hyaluronan-degrading enzymes also include enzymes that catalyze the cleavage of the β1→6 glycosidic bond in the hyaluronan chain or polymer. Thus, the matrix-associated HA polymers are degraded to small tetrasaccharide and hexasaccharide products. Hyaluronan-degrading enzymes, such as hyaluronidases (e.g. PH20) that degrade hyaluronan can reduce the hyaluronan such that the tissue deflates, the blood vessels expand, and more blood can flow through the site. This results in a diminishment of the interstitial fluid pressure at the tissue site and an associated increase in vascular perfusion. Thus, reducing HA in skeletal muscle of insulin resistant subjects by a hyaluronan-degrading enzyme can improve access of all hormones and metabolites delivered by the vasculature, including insulin and glucose, and improve overall muscle health.

While hyaluronidase has been demonstrated to exhibit anti-diabetogenic effects, these effects have been noted in animal models of type 1 diabetes (Weiss et al. (2000) Proc. Natl. Acad. Sci. U.S.A. 97:285-290). This is the first demonstration that hyaluronan-degrading enzymes, such as hyaluronidase for example PH20, can be used to ameliorate or reduce or prevent insulin resistance, and diseases or conditions associated with insulin resistance, such as type 2 diabetes and cardiovascular disease.

Hence, provided herein are methods or uses of a hyaluronan-degrading enzyme for reversing or preventing insulin resistance in a subject. As shown herein, administration of the hyaluronan-degrading enzyme PH20 to insulin-resistant subjects resulted in a reduction in HA levels in muscle ECM by 80%, and is associated with a decrease in fat mass, a decrease in basal arterial insulin, improved insulin action as assessed by a higher glucose infusion rate (GIR) during a hyperinsulinemic euglycemic clamp, decrease in plasma non-esterified fatty acid (NEFA), and increased insulin signaling. In addition, the effect was not limited to the skeletal muscle, as insulin-induced suppression of liver glucose production also was increased. These results demonstrate that ECM HA is linked to insulin resistance, and that administration of a hyaluronan-degrading enzyme can reverse insulin resistance.

Hyaluronan-degrading enzymes, however, can exhibit a short half-life when administered systemically. For example, unmodified hyaluronan-degrading enzyme typically has a short half-life of enzymatic activity in blood of minutes, generally less than 5 minutes. Since the substrate HA is replaced with a half-life of approximately 5 hours, in order to achieve therapeutic activity extended half-life of a hyaluronan-degrading enzyme is required. Therefore, in the methods and uses herein of treating or reversing insulin resistance, and thereby preventing the onset of associated diseases and conditions, a hyaluronan-degrading enzyme conjugated to a polymer is administered. Such an enzyme exhibits greatly increased half-life of hours (e.g. 5 to 10 hours or more), while maintaining therapeutic efficacy in vivo. For example, it is found herein that administration of a polymer-conjugated hyaluronan-degrading enzyme exhibits sufficient therapeutic activity to remove HA in muscle ECM, which is maintained at a decreased level for up to 72 hours.

HA, however, normally exhibits a robust ability for regeneration as it is rapidly turned over in the body, with, for example, a half-life of 15-20 hours in the skin, due to the action of hyaluronidases. It is shown herein that the ability for resynthesis and regeneration of HA means that acute administration (e.g. one injection) of a polymer-conjugated hyaluronan-degrading enzyme does not affect insulin action. In contrast, chronic administration of a hyaluronan-degrading enzyme over the course of a dosage regime cycle, which effects a chronic reduction in HA and limited to no regeneration or resynthesis of HA, exhibits substantial effects on improving insulin action and reversing insulin resistance. These results show that chronic HA reduction results in a long-term adaptation of the muscle ECM to effect improved muscle vascularization and accessibility of metabolic substances such as glucose and insulin. In addition, HA reduction results in a marked decrease in fat mass. The decrease in fat mass could contribute to reversing insulin resistance.

The following sections describe exemplary polymer-conjugated hyaluronan-degrading enzyme for use in treating or reversing insulin resistance, and in preventing the occurrence of insulin resistant-associated diseases or conditions. Exemplary dosage regimes and methods are described.

### C. HYALURONAN-DEGRADING ENZYMES AND POLYMER-CONJUGATED HYALURONAN-DEGRADING ENZYMES

Provided herein are methods and uses of a hyaluronan-degrading enzyme for reducing or ameliorating insulin resistance, where the hyaluronan-degrading enzyme is modified to exhibit increased half-life compared to the hyaluronan-degrading enzyme that is not so modified. For example, the hyaluronan-degrading enzyme for use in the methods herein exhibits a half-life of at least 1 hour, and generally at least 2 hours, 3 hours, 4 hours, 5 hours, 6 hours, 7 hours, 8 hours, 9 hours, 10 hours, 11 hours, 12 hours or more. Generally, the provided compositions for use in the methods contain a hyaluronan-degrading enzyme, in particular a hyaluronidase, such as a soluble hyaluronidases (e.g. a PH20 or truncated PH20), that has been modified by conjugation to one or more polymeric molecule (polymer). The effect of polymer conjugation, such as with a PEG moiety, increases the half-life of the hyaluronan-degrading enzyme, for example, to promote prolonged/sustained treatment effects in a subject.

Hyaluronan (HA or hyaluronic acid) is a component of the extracellular matrix and a major constituent of the interstitial barrier. Hyaluronan-degrading enzymes, such as hyaluronidases, are a family of enzymes that degrade hyaluronic acid, which is an essential component of the extracellular matrix and a major constituent of the interstitial barrier. Hyaluronan-degrading enzymes act to degrade hyaluronan by cleaving hyaluronan polymers, which are composed of repeating disaccharides units, D-glucuronic acid (GlcA) and N-acetyl-D-glucosamine (GlcNAc), linked together via alternating β-1→4 and β1→3 glycosidic bonds. Hyaluronan chains can reach about 25,000 disaccharide repeats or more in length and polymers of hyaluronan can range in size from about 5,000 to 20,000,000 Da in vivo. By catalyzing the hydrolysis of hyaluronic acid, a major constituent of the interstitial barrier, hyaluronan-degrading enzymes lower the viscosity of hyaluronic acid, thereby increasing tissue permeability.

Accordingly, hyaluronan-degrading enzymes for the uses and methods provided herein include any enzyme having the ability to catalyze the cleavage of a hyaluronan disaccharide chain or polymer. In some examples, the hyaluronan-degrading enzyme cleaves the β-1→4 glycosidic bond in the hyaluronan chain or polymer. In other examples, the hyaluronan-degrading enzyme catalyzes the cleavage of the β1→6 glycosidic bond in the hyaluronan chain or polymer.

Hyaluronan-degrading enzymes include hyaluronidases, as well as other enzymes such as chondrotinases and lyases that have the ability to cleave hyaluronan. Further, hyaluronan-degrading enzymes also include soluble forms thereof that can be expressed and secreted from cells. As described below, hyaluronan-degrading enzymes exist in membrane-bound or soluble forms that are secreted from cells. For purposes herein, soluble hyaluronan-degrading enzymes are provided for use in the methods and uses herein. Thus, where hyaluronan-degrading enzymes include a glycosylphosphatidylinositol (GPI) anchor and/or are otherwise membrane-anchored or insoluble, such hyaluronan-degrading enzymes can be provided in soluble form by truncation or deletion of the GPI anchor to render the enzyme secreted and soluble. Thus, hyaluronan-degrading enzymes include truncated variants, e.g. truncated to remove all or a portion of a GPI anchor. Exemplary of such soluble hyaluronidases are soluble PH20 hyaluronides, such as any set forth in U.S. Pat. No. 7,767,429; U.S. Publication Nos. US 2004/0268425 or US 2010/0143457.

Hyaluronan-degrading enzymes provided herein also include variants of any hyaluronan-degrading enzyme, such as any hyaluronidase or soluble hyaluronidase, for example a PH20, that is known to one of skill in the art or described herein. For example, hyaluronan-degrading enzymes can contain one or more variations in its primary sequence, such as amino acid substitutions, additions and/or deletions. A variant of a hyaluronan-degrading enzyme generally exhibits at least or about 60%, 70%, 80%, 90%, 91%, 92%, 93%, 94%, 95%, 96%, 97%, 98%, 99% or more sequence identity compared to the hyaluronan-degrading enzyme not containing the variation. Any variation can be included in the hyaluronan-degrading enzyme for the purposes herein, provided the enzyme retains hyaluronidase activity, such as at least or about 5%, 10%, 15%, 20%, 25%, 30%, 35%, 40%, 45%, 50%, 55%, 60%, 65%, 70%, 75%, 80%, 85%, 90%, 95% or more of the activity of a hyaluronan-degrading enzyme not containing the variation (as measured by in vitro and/or in vivo assays well known in the art and described herein). For example, exemplary of hyaluronan-degrading enzymes, including those that can be conjugated to a polymer, are any set forth in any of SEQ ID NOS:1, 2, 4-9, 47, 48, 150-170, 183-189 and 199-210, or any that exhibit at least or about 60%, 70%, 80%, 90%, 91%, 92%, 93%, 94%, 95%, 96%, 97%, 98%, 99% or more sequence identity to any of SEQ ID NOS:1, 2, 4-9, 47, 48, 150-170, 183-189 and 199-210.

Various forms of hyaluronan-degrading enzymes, including hyaluronidases, have been prepared and approved for therapeutic use in subjects, including humans. For example, animal-derived hyaluronidase preparations include Vitrase® (ISTA Pharmaceuticals), a purified ovine testicular hyaluronidase, Amphadase® (Amphastar Pharmaceuticals), a bovine testicular hyaluronidase and Hydase™ (Prima Pharm Inc.), a bovine testicular hyaluronidase. Hylenex® (Halozyme Therapeutics) is a human recombinant hyaluronidase produced by genetically engineered Chinese Hamster Ovary (CHO) cells containing nucleic acid encoding soluble forms of PH20, designated rHuPH20 (see, e.g. U.S. Publication No. US 2004/0268425 and U.S. Pat. No. 7,767,429). It is understood that any hyaluronidase preparation can be used in the combinations, methods and uses provided herein (see, e.g. U.S. Pat. Nos. 2,488,564; 2,488,565; 2,676,139; 2,795,529; 2,806,815, 2,808,362; 5,747,027; 5,827,721; 7,767,429; U.S. Publication No. US 2004/0268425; and International PCT Application No. WO 2005/118799), or any provided herein.

A non-limiting description of exemplary hyaluronan-degrading enzymes, such as hyaluronidase enzymes or soluble hyaluronidase enzymes, for example PH20, for use in the methods provided herein are described below. Generally, such hyaluronan-degrading enzymes include those that are conjugated to a polymer.

1. Hyaluronidases

Hyaluronidases are members of a large family of hyaluronan-degrading enzymes. There are three general classes of hyaluronidases: mammalian-type hyaluronidases, bacterial hyaluronidases and hyaluronidases from leeches, other parasites and crustaceans. Such enzymes can be used in the compositions and methods provided herein.

a. Mammalian-Type Hyaluronidases

Mammalian-type hyaluronidases (EC 3.2.1.35) are endo-β-N-acetylhexosaminidases that hydrolyze the β-1→4 glycosidic bond of hyaluronan into various oligosaccharide lengths such as tetrasaccharides and hexasaccharides. These enzymes have both hydrolytic and transglycosidase activities, and can degrade hyaluronan and chondroitin sulfates (CS), generally C4-S and C6-S. Hyaluronidases of this type include, but are not limited to, from cows (bovine) (SEQ ID NOS:10, 11, 64, 203 and 204 and nucleic acid molecules set forth in SEQ ID NOS:190-192), sheep (Ovis aries) (SEQ ID NO:26, 27, 63 and 65, nucleic acid molecules set forth in SEQ ID NOS:66 and 193-194), yellow jacket wasp (SEQ ID NOS:12 and 13), honey bee (SEQ ID NO:14), white-face hornet (SEQ ID NO:15), paper wasp (SEQ ID NO:16), mouse (SEQ ID NOS:17-19, 32, 205), pig (SEQ ID NOS:20-21), rat (SEQ ID NOS:22-24, 31, 206), rabbit (SEQ ID NO:25, 207), orangutan (SEQ ID NO:28), cynomolgus monkey (SEQ ID NO:29, 202), guinea pig (SEQ ID NO:30, 208), chimpanzee (SEQ ID NO:101, 199, 200), rhesus monkey (SEQ ID NO:102, 201), fox (SEQ ID NO:209 and 210) and human hyaluronidases (SEQ ID NOS:1-2, 36-39). The above hyaluronidases include PH20 hyaluronidases. Also, BH55 hyaluronidase is of this type as described in U.S. Pat. Nos. 5,747,027 and 5,827,721. Exemplary of hyaluronidases in the compositions and methods provided herein are soluble hyaluronidases.

Mammalian hyaluronidases can be further subdivided into those that are neutral active, predominantly found in testes extracts, and acid active, predominantly found in organs such as the liver. Exemplary neutral active hyaluronidases include PH20, including but not limited to, PH20 derived from different species such as ovine (SEQ ID NOS:27, 63 and 65), bovine (SEQ ID NO:11 and 64) and human (SEQ ID NO:1). Human PH20 (also known as SPAM 1 or sperm surface protein PH20), is generally attached to the plasma membrane via a glycosylphosphatidyl inositol (GPI) anchor. It is naturally involved in sperm-egg adhesion and aids penetration by sperm of the layer of cumulus cells by digesting hyaluronic acid.

Besides human PH20 (also termed SPAM 1), five hyaluronidase-like genes have been identified in the human genome, HYAL1, HYAL2, HYAL3, HYAL4 and HYALP1. HYALP1 is a pseudogene, and HYAL3 (SEQ ID NO:38) has not been shown to possess enzyme activity toward any known substrates. HYAL4 (precursor polypeptide set forth in SEQ ID NO:39) is a chondroitinase and exhibits little activity towards hyaluronan. HYAL1 (precursor polypeptide set forth in SEQ ID NO:36) is the prototypical acid-active enzyme and PH20 (precursor polypeptide set forth in SEQ ID NO:1) is the prototypical neutral-active enzyme. Acid-active hyaluronidases, such as HYAL1 and HYAL2 (precursor polypeptide set forth in SEQ ID NO:37) generally lack catalytic activity at neutral pH (i.e. pH 7). For example, HYAL1 has little catalytic activity in vitro over pH 4.5 (Frost et al. (1997) Anal. Biochem. 251:263-269). HYAL2 is an acid-active enzyme with a very low specific activity in vitro. The hyaluronidase-like enzymes also can be characterized by those which are generally attached to the plasma membrane via a glycosylphosphatidyl inositol (GPI) anchor such as human HYAL2 and human PH20 (Danilkovitch-Miagkova et al. (2003) Proc. Natl. Acad. Sci. U.S.A. 100(8):4580-4585), and those which are generally soluble such as human HYAL1 (Frost et al. (1997) Biochem. Biophys. Res. Commun. 236(1):10-15).

### PH20

PH20, like other mammalian hyaluronidases, is an endo-β-N-acetyl-hexosaminidase that hydrolyzes the β1→4 glycosidic bond of hyaluronic acid into various oligosaccharide lengths such as tetrasaccharides and hexasaccharides. It has both hydrolytic and transglycosidase activities and can degrade hyaluronic acid and chondroitin sulfates, such as C4-S and C6-S. PH20 is naturally involved in sperm-egg adhesion and aids penetration by sperm of the layer of cumulus cells by digesting hyaluronic acid. PH20 is located on the sperm surface, and in the lysosome-derived acrosome, where it is bound to the inner acrosomal membrane. Plasma membrane PH20 has hyaluronidase activity only at neutral pH, while inner acrosomal membrane PH20 has activity at both neutral and acid pH. In addition to being a hyaluronidase, PH20 also appears to be a receptor for HA-induced cell signaling, and a receptor for the zona pellucida surrounding the oocyte.

Exemplary PH20 proteins, including precursor and mature forms, include, but are not limited to, human (precursor polypeptide set forth in SEQ ID NO:1, mature polypeptide set forth in SEQ ID NO:2), chimpanzee (SEQ ID NO:101, 199, 200), rhesus monkey (SEQ ID NO:102, 201) bovine (SEQ ID NOS:11 and 64, 203, 204), rabbit (SEQ ID NO:25, 207), ovine PH20 (SEQ ID NOS:27, 63 and 65), cynomolgus monkey (SEQ ID NO:29, 202), guinea pig (SEQ ID NO:30, 208), rat (SEQ ID NO:31, 206), mouse (SEQ ID NO:32, 205) and fox (SEQ ID NO:209 and 210) PH20 polypeptides.

Bovine PH20 is a 553 amino acid precursor polypeptide (SEQ ID NO:11). Alignment of bovine PH20 with the human PH20 shows only weak homology, with multiple gaps existing from amino acid 470 through to the respective carboxy termini due to the absence of a GPI anchor in the bovine polypeptide (see, e.g. Frost (2007) Expert Opin. Drug. Deliv. 4:427-440). In fact, clear GPI anchors are not predicted in many other PH20 species besides humans. Thus, PH20 polypeptides produced from ovine and bovine naturally exist as soluble forms. Though bovine PH20 exists very loosely attached to the plasma membrane, it is not anchored via a phospholipase sensitive anchor (Lalancette et al. (2001) Biol. Reprod. 65(2):628-636). This unique feature of bovine hyaluronidase has permitted the use of the soluble bovine testes hyaluronidase enzyme as an extract for clinical use (Wydase®, Hyalase®).

The human PH20 mRNA transcript is normally translated to generate a 509 amino acid precursor polypeptide (SEQ ID NO:1) containing a 35 amino acid signal sequence at the N-terminus (amino acid residue positions 1-35) and a 19 amino acid glycosylphosphatidylinositol (GPI) anchor attachment signal sequence at the C-terminus (amino acid residue positions 491-509). The mature PH20 therefore, is a 474 amino acid polypeptide set forth in SEQ ID NO:2. Following transport of the precursor polypeptide to the ER and removal of the signal peptide, the C-terminal GPI-attachment signal peptide is cleaved to facilitate covalent attachment of a GPI anchor to the newly-formed C-terminal amino acid at the amino acid position corresponding to position 490 of the precursor polypeptide set forth in SEQ ID NO:1. Thus, a 474 amino acid GPI-anchored mature polypeptide with an amino acid sequence set forth in SEQ ID NO:2 is produced.

Human PH20 exhibits hyaluronidase activity at neutral and acid pH. In one aspect, human PH20 is the prototypical neutral-active hyaluronidase that is generally locked to the plasma membrane via a GPI anchor. In another aspect, PH20 is expressed on the inner acrosomal membrane where it has hyaluronidase activity at neutral and acid pH. It appears that PH20 contains two catalytic sites at distinct regions of the polypeptide: the Peptide 1 and Peptide 3 regions (Chem et al. (2001) Matrix Biol. 20:515-525). Evidence indicates that the Peptide 1 region of PH20, which corresponds to amino acid positions 107-137 of the mature polypeptide set forth in SEQ ID NO:2 and positions 142-172 of the precursor polypeptide set forth in SEQ ID NO:1, is required for enzyme activity at neutral pH. Amino acids at positions 111 and 113 (corresponding to the mature PH20 polypeptide set forth in SEQ ID NO:2) within this region appear to be important for activity, as mutagenesis by amino acid replacement results in PH20 polypeptides with 3% hyaluronidase activity or undetectable hyaluronidase activity, respectively, compared to the wild-type PH20 (Arming et al. (1997) Eur. J. Biochem. 247:810-814).

The Peptide 3 region, which corresponds to amino acid positions 242-262 of the mature polypeptide set forth in SEQ ID NO:2, and positions 277-297 of the precursor polypeptide set forth in SEQ ID NO:1, appears to be important for enzyme activity at acidic pH. Within this region, amino acids at positions 249 and 252 of the mature PH20 polypeptide appear to be essential for activity, and mutagenesis of either one results in a polypeptide essentially devoid of activity (Arming et al. (1997) Eur. J. Biochem. 247:810-814).

In addition to the catalytic sites, PH20 also contains a hyaluronan-binding site. Experimental evidence indicate that this site is located in the Peptide 2 region, which corresponds to amino acid positions 205-235 of the precursor polypeptide set forth in SEQ ID NO:1 and positions 170-200 of the mature polypeptide set forth in SEQ ID NO:2. This region is highly conserved among hyaluronidases and is similar to the heparin binding motif. Mutation of the arginine residue at position 176 (corresponding to the mature PH20 polypeptide set forth in SEQ ID NO:2) to a glycine results in a polypeptide with only about 1% of the hyaluronidase activity of the wild type polypeptide (Arming et al. (1997) Eur. J. Biochem. 247:810-814).

There are seven potential N-linked glycosylation sites in human PH20 at N82, N166, N235, N254, N368, N393, N490 of the polypeptide exemplified in SEQ ID NO:1. Because amino acids 36 to 464 of SEQ ID NO:1 appear to contain the minimally active human PH20 hyaluronidase domain, the N-linked glycosylation site N-490 is not required for proper hyaluronidase activity. There are six disulfide bonds in human PH20. Two disulfide bonds between the cysteine residues C60 and C351 and between C224 and C238 of the polypeptide exemplified in SEQ ID NO:1 (corresponding to residues C25 and C316, and C189 and C203 of the mature polypeptide set forth in SEQ ID NO:2, respectively). A further four disulfide bonds are formed between between the cysteine residues C376 and C387; between C381 and C435; between C437 and C443; and between C458 and C464 of the polypeptide exemplified in SEQ ID NO:1 (corresponding to residues C341 and C352; between C346 and C400; between C402 and C408; and between C423 and C429 of the mature polypeptide set forth in SEQ ID NO:2, respectively).

b. Bacterial Hyaluronidases

Bacterial hyaluronidases (EC 4.2.2.1 or EC 4.2.99.1) degrade hyaluronan and, to various extents, chondroitin sulfates and dermatan sulfates. Hyaluronan lyases isolated from bacteria differ from hyaluronidases from other sources (e.g. hyaluronoglucosaminidases, EC 3.2.1.35) by their mode of action. They are endo-β-N-acetylhexosaminidases that catalyze an elimination reaction, rather than hydrolysis, of the β1→4-glycosidic linkage between N-acetyl-beta-D-glucosamine and D-glucuronic acid residues in hyaluronan, yielding 3-(4-deoxy-β-D-gluc-4-enuronosyl)-N-acetyl-D-glucosamine tetra- and hexasaccharides, and disaccharide end products. The reaction results in the formation of oligosaccharides with unsaturated hexuronic acid residues at their nonreducing ends.

Exemplary hyaluronidases from bacteria for use in the compositions, combinations and methods provided include, but are not limited to, hyaluronan-degrading enzymes in microorganisms, including strains of Arthrobacter, Bdellovibrio, Clostridium, Micrococcus, Streptococcus, Peptococcus, Propionibacterium. Bacteroides, and Streptomyces. Particular examples of such strains and enzymes include, but are not limited to Arthrobacter sp. (strain FB24) (SEQ ID NO:67), Bdellovibrio bacteriovorus (SEQ ID NO:68), Propionibacterium acnes (SEQ ID NO:69), Streptococcus agalactiae ((SEQ ID NO:70); 18RS21 (SEQ ID NO:71); serotype Ia (SEQ ID NO:72); serotype III (SEQ ID NO:73), Staphylococcus aureus (strain COL (SEQ ID NO:74); strain MRSA252 (SEQ ID NOS:75 and 76); strain MSSA476 (SEQ ID NO:77); strain NCTC 8325 (SEQ ID NO:78); strain bovine RF122 (SEQ ID NOS:79 and 80); strain USA300 (SEQ ID NO:81), Streptococcus pneumoniae ((SEQ ID NO:82); strain ATCC BAA-255/R6 (SEQ ID NO:83); serotype 2, strain D39/NCTC 7466 (SEQ ID NO:84), Streptococcus pyogenes (serotype M1) (SEQ ID NO:85); serotype M2, strain MGAS 10270 (SEQ ID NO:86); serotype M4, strain MGAS 10750 (SEQ ID NO:87); serotype M6 (SEQ ID NO:88); serotype M12, strain MGAS2096 (SEQ ID NOS:89 and 90); serotype M12, strain MGAS9429 (SEQ ID NO:91); serotype M28 (SEQ ID NO:92); Streptococcus suis (SEQ ID NOS:93-95); Vibrio fischeri (strain ATCC 700601/ESI 14 (SEQ ID NO:96)), and the Streptomyces hyaluronolyticus hyaluronidase enzyme, which is specific for hyaluronic acid and does not cleave chondroitin or chondroitin sulfate (Ohya and Kaneko (1970) Biochim. Biophys. Acta 198:607).

c. Hyaluronidases from Leeches, Other Parasites and Crustaceans

Hyaluronidases from leeches, other parasites, and crustaceans (EC 3.2.1.36) are endo-β-glucuronidases that generate tetra- and hexasaccharide end-products. These enzymes catalyze hydrolysis of 1→3-linkages between β-D-glucuronate and N-acetyl-D-glucosamine residues in hyaluronate. Exemplary hyaluronidases from leeches include, but are not limited to, hyaluronidase from Hirudinidae (e.g. Hirudo medicinalis), Erpobdellidae (e.g. Nephelopsis obscura and Erpobdella punctata,), Glossiphoniidae (e.g. Desserobdella pitta, Helobdella stagnalis, Glossiphonia complanata, Placobdella ornata and Theromyzon sp.) and Haemopidae (Haemopis marmorata) (Hovingh et al. (1999) Comp. Biochem. Physiol. B Biochem. Mol. Biol. 124(3):319-26). An exemplary hyaluronidase from bacteria that has the same mechanism of action as the leech hyaluronidase is that from the cyanobacteria, Synechococcus sp. (strain RCC307, SEQ ID NO:97).

2. Other Hyaluronan-Degrading Enzymes

In addition to the hyaluronidase family, other hyaluronan-degrading enzymes can be used in the compositions and methods provided. For example, enzymes, including particular chondroitinases and lyases, that have the ability to cleave hyaluronan can be employed. Exemplary chondroitinases that can degrade hyaluronan include, but are not limited to, chondroitin ABC lyase (also known as chondroitinase ABC), chondroitin AC lyase (also known as chondroitin sulfate lyase or chondroitin sulfate eliminase) and chondroitin C lyase. Methods for production and purification of such enzymes for use in the compositions, combinations, and methods provided are known in the art (e.g. U.S. Pat. No. 6,054,569; Yamagata et al. (1968) J. Biol. Chem. 243(7):1523-1535; Yang et al. (1985) J. Biol. Chem. 160(30):1849-1857).

Chondroitin ABC lyase contains two enzymes, chondroitin-sulfate-ABC endolyase (EC 4.2.2.20) and chondroitin-sulfate-ABC exolyase (EC 4.2.2.21) (Hamai et al. (1997) J. Biol. Chem. 272(14):9123-30), which degrade a variety of glycosaminoglycans of the chondroitin-sulfate- and dermatan-sulfate type. Chondroitin sulfate, chondroitin-sulfate proteoglycan and dermatan sulfate are the preferred substrates for chondroitin-sulfate-ABC endolyase, but the enzyme also can act on hyaluronan at a lower rate. Chondroitin-sulfate-ABC endolyase degrades a variety of glycosaminoglycans of the chondroitin-sulfate- and dermatan-sulfate type, producing a mixture of Δ4-unsaturated oligosaccharides of different sizes that are ultimately degraded to Δ4-unsaturated tetra- and disaccharides. Chondroitin-sulfate-ABC exolyase has the same substrate specificity but removes disaccharide residues from the non-reducing ends of both polymeric chondroitin sulfates and their oligosaccharide fragments produced by chondroitin-sulfate-ABC endolyase (Hamai et al. (1997) J. Biol. Chem. 272:9123-9130). Exemplary chondroitin-sulfate-ABC endolyases and chondroitin-sulfate-ABC exolyases include, but are not limited to, those from Proteus vulgaris and Flavobacterium heparinum (the Proteus vulgaris chondroitin-sulfate-ABC endolyase is set forth in SEQ ID NO:98 (Sato et al. (1994) Appl. Microbiol. Biotechnol. 41(1):39-46).

Chondroitin AC lyase (EC 4.2.2.5) is active on chondroitin sulfates A and C, chondroitin and hyaluronic acid, but is not active on dermatan sulfate (chondroitin sulfate B). Exemplary chondroitinase AC enzymes from the bacteria include, but are not limited to, those from Flavobacterium heparinum and Victivallis vadensis, set forth in SEQ ID NOS:99 and 100, respectively, and Arthrobacter aurescens (Tkalec et al. (2000) Appl. Environ. Microbiol. 66(1):29-35; Ernst et al. (1995) Crit. Rev. Biochem. Mol. Biol. 30(5):387-444).

Chondroitinase C cleaves chondroitin sulfate C producing tetrasaccharide plus an unsaturated 6-sulfated disaccharide (delta Di-6S). It also cleaves hyaluronic acid producing unsaturated non-sulfated disaccharide (delta Di-OS). Exemplary chondroitinase C enzymes from the bacteria include, but are not limited to, those from Streptococcus and Flavobacterium (Hibi et al. (1989) FEMS Microbiol. Lett. 48(2):121-4; Michelacci et al. (1976) J. Biol. Chem. 251:1154-8; Tsuda et al. (1999) Eur. J. Biochem. 262:127-133).

3. Soluble Hyaluronan-Degrading Enzymes

Provided in the compositions, uses and methods herein are soluble hyaluronan-degrading enzymes, including soluble hyaluronidases. Soluble hyaluronan-degrading enzymes include any hyaluronan-degrading enzymes that are secreted from cells (e.g. CHO cells) upon expression and exist in soluble form. Such enzymes include, but are not limited to, soluble hyaluronidases, including non-human soluble hyaluronidases, including non-human animal soluble hyaluronidases, bacterial soluble hyaluronidases and human hyaluronidases, Hyal1, bovine PH20 and ovine PH20, allelic variants thereof and other variants thereof. For example, included among soluble hyaluronan-degrading enzymes are any hyaluronan-degrading enzymes that have been modified to be soluble. For example, hyaluronan-degrading enzymes that contain a GPI anchor can be made soluble by truncation of and removal of all or a portion of the GPI anchor. In one example, the human hyaluronidase PH20, which is normally membrane-anchored via a GPI anchor, can be made soluble by truncation of and removal of all or a portion of the GPI anchor at the C-terminus.

Soluble hyaluronan-degrading enzymes also include neutral active and acid active hyaluronidases. Depending on factors such as, but not limited to, the desired level of activity of the enzyme following administration and/or site of administration, neutral active and acid active hyaluronidases can be selected. In a particular example, the hyaluronan-degrading enzyme for use in the compositions and methods herein is a soluble neutral active hyaluronidase.

Exemplary of a soluble hyaluronidase is PH20 from any species, such as any set forth in any of SEQ ID NOS:1, 2, 11, 25, 27, 29-32, 63-65 and 101-102, or truncated forms thereof lacking all or a portion of the C-terminal GPI anchor, so long as the hyaluronidase is soluble (secreted upon expression) and retains hyaluronidase activity. Also included among soluble hyaluronidases are variants of any of SEQ ID NOS:1, 2, 11, 25, 27, 29-32, 63-65 and 101-102, or truncated forms thereof, that exhibit hyaluronidase activity. Variants include polypeptides having 60%, 70%, 80%, 90%, 91%, 92%, 93%, 94%, 95%, 96%., 97%, 98%, 99% or more sequence identity to any of SEQ ID NOS:1, 2, 11, 25, 27, 29-32, 63-65 and 101-102, mature (e.g. lacking the signal sequence) or truncated forms thereof. Amino acid variants include conservative and non-conservative mutations. It is understood that residues that are important or otherwise required for the activity of a hyaluronidase, such as any described above or known to those of skill in the art, are generally invariant and cannot be changed. These include, for example, active site residues. Thus, for example, amino acid residues 111, 113 and 176 (corresponding to residues in the mature PH20 polypeptide set forth in SEQ ID NO:2) of a human PH20 polypeptide, or soluble form thereof, are generally invariant and are not altered. Other residues that confer glycosylation and formation of disulfide bonds required for proper folding also can be invariant.

In some instances, the soluble hyaluronan-degrading enzyme is normally GPI-anchored (such as, for example, human PH20) and is rendered soluble by truncation at the C-terminus. Such truncation can remove all of the GPI anchor attachment signal sequence, or can remove only some of the GPI anchor attachment signal sequence. The resulting polypeptide, however, is soluble. In instances where the soluble hyaluronan-degrading enzyme retains a portion of the GPI anchor attachment signal sequence, 1, 2, 3, 4, 5, 6, 7 or more amino acid residues in the GPI-anchor attachment signal sequence can be retained, provided the polypeptide is soluble. Polypeptides containing one or more amino acids of the GPI anchor are termed extended soluble hyaluronan-degrading enzymes. One of skill in the art can determine whether a polypeptide is GPI-anchored using methods well known in the art. Such methods include, but are not limited to, using known algorithms to predict the presence and location of the GPI-anchor attachment signal sequence and ω-site, and performing solubility analyses before and after digestion with phosphatidylinositol-specific phospholipase C(PI-PLC) or D (PI-PLD).

Extended soluble hyaluronan-degrading enzymes can be produced by making C-terminal truncations to any naturally GPI-anchored hyaluronan-degrading enzyme such that the resulting polypeptide is soluble and contains one or more amino acid residues from the GPI-anchor attachment signal sequence (see, e.g. U.S. Pub. No. US 2010/0143457). Exemplary extended soluble hyaluronan-degrading enzymes that are C-terminally truncated but retain a portion of the GPI anchor attachment signal sequence include, but are not limited to, extended soluble PH20 (esPH20) polypeptides of primate origin, such as, for example, human and chimpanzee esPH20 polypeptides. For example, the esPH20 polypeptides can be made by C-terminal truncation of any of the mature or precursor polypeptides set forth in SEQ ID NOS:1, 2 or 101, or other variants thereof, including active fragment thereof, wherein the resulting polypeptide is soluble and retains one or more amino acid residues from the GPI-anchor attachment signal sequence. Variants include polypeptides having 60%, 70%, 80%, 90%, 91%, 92%, 93%, 94%, 95% or more sequence identity to any of SEQ ID NOS:1 or 2 and that retain hyaluronidase activity. The esPH20 polypeptides provided herein can be C-terminally truncated by 1, 2, 3, 4, 5, 6, 7, 8, 9, 10 or more amino acids compared to the wild type polypeptide, such as a polypeptide with a sequence set forth in SEQ ID NOS:1, 2 or 101, provided the resulting esPH20 polypeptide is soluble and retains 1 or more amino acid residues from the GPI-anchor attachment signal sequence.

Typically, for use in the compositions and methods herein, a soluble human hylauronan-degrading enzyme, such as a soluble human PH20, is used. Although hylauronan-degrading enzymes, such as PH20, from other animals can be utilized, such preparations are potentially immunogenic, since they are animal proteins. For example, a significant proportion of patients demonstrate prior sensitization secondary to ingested foods, and since these are animal proteins, all patients have a risk of subsequent sensitization. Thus, non-human preparations may not be suitable for chronic use. If non-human preparations are desired, it is contemplated herein that such polypeptides can be prepared to have reduced immunogenicity. Such modifications are within the level of one of skill in the art and can include, for example, removal and/or replacement of one or more antigenic epitopes on the molecule.

Hyaluronan-degrading enzymes, including hyaluronidases (e.g. PH20), used in the methods herein can be recombinantly produced or can be purified or partially-purified from natural sources, such as, for example, from testes extracts. Methods for production of recombinant proteins, including recombinant hyaluronan-degrading enzymes, are provided elsewhere herein and are well known in the art.

a. Soluble Human PH20

Exemplary of a soluble hyaluronidase is soluble human PH20, Soluble forms of recombinant human PH20 have been produced and can be used in the compositions and methods described herein. The production of such soluble forms of PH20 is described in U.S. Pub. Nos. US 2004/0268425; US 2005/0260186; US 2006/0104968; US 2010/0143457; and International PCT App. No. WO 2009/111066. For example, soluble PH20 polypeptides include C-terminally truncated variant polypeptides that include a sequence of amino acids in SEQ ID NOS:1 or 2, or have at least 91%, 92%, 93%, 94%, 95%, 95%, 97%, 98% sequence identity to a sequence of amino acids included in SEQ ID NO:1 or 2, retain hyaluronidase activity and are soluble. Included among these polypeptides are soluble PH20 polypeptides that completely lack all or a portion of the GPI-anchor attachment signal sequence.

Also included are extended soluble PH20 (esPH20) polypeptides that contain at least one amino acid of the GPI anchor. Thus, instead of having a GPI-anchor covalently attached to the C-terminus of the protein in the ER and being anchored to the extracellular leaflet of the plasma membrane, these polypeptides are secreted and are soluble. C-terminally truncated PH20 polypeptides can be C-terminally truncated by 1, 2, 3, 4, 5, 6, 7, 8, 9, 10, 11, 12, 13, 14, 15, 16, 17, 18, 19, 20, 25, 30, 35, 40, 45, 50, 55, 60 or more amino acids compared to the full length wild type polypeptide, such as a full length wild type polypeptide with a sequence set forth in SEQ ID NOS:1 or 2, or allelic or species variants or other variants thereof.

For example, soluble forms include, but are not limited to, C-terminal truncated polypeptides of human PH20 set forth in SEQ ID NO:1 having a C-terminal amino acid residue 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO:1, or polypeptides that exhibit at least 85% identity thereto. Soluble forms of human PH20 generally include those that contain amino acids 36-464 set forth in SEQ ID NO:1. For example, when expressed in mammalian cells, the 35 amino acid N-terminal signal sequence is cleaved during processing, and the mature form of the protein is secreted. Thus, the mature soluble polypeptides contain amino acids 36 to 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of SEQ ID NO:1. Table 3 provides non-limiting examples of exemplary C-terminally truncated PH20 polypeptides, including C-terminally truncated soluble PH20 polypeptides. In Table 3 below, the length (in amino acids) of the precursor and mature polypeptides, and the sequence identifier (SEQ ID NO) in which exemplary amino acid sequences of the precursor and mature polypeptides of the C-terminally truncated PH20 proteins are set forth, are provided. The wild-type PH20 polypeptide also is included in Table 3 for comparison. In particular, exemplary of soluble hyaluronidases are soluble human PH20 polypeptides that are 442, 443, 444, 445, 446 or 447 amino acids in length, such as set forth in any of SEQ ID NOS:4-9, or allelic or species variants or other variants thereof.

TABLE 3
Exemplary C-terminally truncated PH20 polypeptides

| | | | | Mature |
|---|---|---|---|---|
| | Precursor | Precursor | Mature | SEQ |
| Polypeptide | (amino acids) | SEQ ID NO | (amino acids) | ID NO |
| wildtype | 509 | 1 | 474 | 2 |
| SPAM1-SILF | 500 | 139 | 465 | 183 |
| SPAM-VSIL | 499 | 106 | 464 | 150 |
| SPAM1-IVSI | 498 | 140 | 463 | 184 |
| SPAM1-FIVS | 497 | 107 | 462 | 151 |
| SPAM1-MFIV | 496 | 141 | 461 | 185 |
| SPAM1-TMFI | 495 | 108 | 460 | 152 |
| SPAM1-ATMF | 494 | 142 | 459 | 186 |
| SPAM1-SATM | 493 | 109 | 458 | 153 |
| SPAM1-LSAT | 492 | 143 | 457 | 187 |
| SPAM1-TLSA | 491 | 110 | 456 | 154 |
| SPAM1-PSTL | 489 | 111 | 454 | 155 |
| SPAM1-SPST | 488 | 144 | 453 | 188 |
| SPAM1-STLS | 490 | 112 | 455 | 156 |
| SPAM1-ASPS | 487 | 113 | 452 | 157 |
| SPAM1-NASP | 486 | 145 | 451 | 189 |
| SPAM1-YNAS | 485 | 114 | 450 | 158 |
| SPAM1-FYNA | 484 | 115 | 449 | 159 |
| SPAM1-IFYN | 483 | 46 | 448 | 48 |
| SPAM1-QIFY | 482 | 3 | 447 | 4 |
| SPAM1-PQIF | 481 | 45 | 446 | 5 |
| SPAM1-EPQI | 480 | 44 | 445 | 6 |
| SPAM1-EEPQ | 479 | 43 | 444 | 7 |
| SPAM1-TEEP | 478 | 42 | 443 | 8 |
| SPAM1-ETEE | 477 | 41 | 442 | 9 |
| SPAM1-METE | 476 | 116 | 441 | 160 |
| SPAM1-PMET | 475 | 117 | 440 | 161 |
| SPAM1-PPME | 474 | 118 | 439 | 162 |
| SPAM1-KPPM | 473 | 119 | 438 | 163 |
| SPAM1-LKPP | 472 | 120 | 437 | 164 |
| SPAM1-FLKP | 471 | 121 | 436 | 165 |
| SPAM1-AFLK | 470 | 122 | 435 | 166 |
| SPAM1-DAFL | 469 | 123 | 434 | 167 |
| SPAM1-IDAF | 468 | 124 | 433 | 168 |
| SPAM1-CIDA | 467 | 40 | 432 | 47 |
| SPAM1-VCID | 466 | 125 | 431 | 169 |
| SPAM1-GVCI | 465 | 126 | 430 | 170 |

Generally, soluble forms of PH20 are produced using protein expression systems that facilitate correct N-glycosylation to ensure the polypeptide retains activity, since glycosylation is important for the catalytic activity and stability of hyaluronidases. Such cells include, for example, Chinese Hamster Ovary (CHO) cells (e.g. DG44 CHO cells). Other C-terminally truncated PH20 polypeptides, including precursor and mature forms, are set forth in any of SEQ ID NOS: 103-105, 127-138, 146-149 and 171-182.

b. rHuPH20

Recombinant soluble forms of human PH20 have been generated and can be used in the compositions and methods provided herein. The generation of such soluble forms of recombinant human PH20 are described, for example, in U.S. Pub. Nos. US 2004/0268425; US 2005/0260186; US 2006/0104968; US 2010/0143457; and International PCT App. No. WO 2009/111066. Exemplary of such polypeptides are those generated by expression of a nucleic acid molecule encoding amino acids 1-482 (set forth in SEQ ID NO:3). Such an exemplary nucleic acid molecule is set forth in SEQ ID NO:49. Post translational processing removes the 35 amino acid signal sequence, leaving a 447 amino acid soluble recombinant human PH20 (SEQ ID NO:4). As produced in the culture medium there is heterogeneity at the C-terminus such that the product, designated rHuPH20, includes a mixture of species that can include any one or more of SEQ ID NOS:4-9 in various abundance. Typically, rHuPH20 is produced in cells that facilitate correct N-glycosylation to retain activity, such as CHO cells (e.g. DG44 CHO cells).

4. Glycosylation of Hyaluronan-Degrading Enzymes

Glycosylation, including N- and O-linked glycosylation, of some hyaluronan-degrading enzymes, including hyaluronidases, can be important for their catalytic activity and stability. While altering the type of glycan modifying a glycoprotein can have dramatic affects on a protein's antigenicity, structural folding, solubility, and stability, most enzymes are not thought to require glycosylation for optimal enzyme activity. For some hyaluronidases, removal of N-linked glycosylation can result in near complete inactivation of the hyaluronidase activity. Thus, for such hyaluronidases, the presence of N-linked glycans is critical for generating an active enzyme.

N-linked oligosaccharides fall into several major types (oligomannose, complex, hybrid, sulfated), all of which have (Man) 3-GlcNAc-GlcNAc-cores attached via the amide nitrogen of Asn residues that fall within -Asn-Xaa-Thr/Ser-sequences (where Xaa is not Pro). Glycosylation at an -Asn-Xaa-Cys-site has been reported for coagulation protein C. In some instances, a hyaluronan-degrading enzyme, such as a hyaluronidase, can contain both N-glycosidic and O-glycosidic linkages. For example, PH20 has O-linked oligosaccharides as well as N-linked oligosaccharides. There are seven potential N-linked glycosylation sites at N82, N166, N235, N254, N368, N393, N490 of human PH20 exemplified in SEQ ID NO:1. Amino acid residues N82, N166 and N254 are occupied by complex type glycans whereas amino acid residues N368 and N393 are occupied by high mannose type glycans. Amino acid residue N235 is occupied by approximately 80% high mannose-type glycans and 20% complex-type glycans. As noted above, N-linked glycosylation at N490 is not required for hyaluronidase activity.

In some examples, the hyaluronan-degrading enzymes for use in the compositions and methods provided are glycosylated at one or all of the glycosylation sites. For example, for human PH20 or a soluble form thereof, 2, 3, 4, 5, or 6 of the N-glycosylation sites corresponding to amino acids N82, N166, N235, N254, N368, and N393 of SEQ ID NO:1 are glycosylated. In some examples, the hyaluronan-degrading enzymes are glycosylated at one or more native glycosylation sites. In other examples, the hyaluronan-degrading enzymes are modified at one or more non-native glycosylation sites to confer glycosylation of the polypeptide at one or more additional sites. In such examples, attachment of additional sugar moieties can enhance the pharmacokinetic properties of the molecule, such as improved half-life and/or improved activity.

In other examples, the hyaluronan-degrading enzymes for use in the compositions and methods provided herein are partially deglycosylated (or N-partially glycosylated polypeptides). For example, partially deglycosylated soluble PH20 polypeptides that retain all or a portion of the hyaluronidase activity of a fully glycosylated hyaluronidase can be used in the compositions, combinations and/or methods provided herein. Exemplary partially deglycosylated hyalurodinases include soluble forms of partially deglycosylated PH20 polypeptides from any species, such as any set forth in any of SEQ ID NOS:1, 2, 11, 25, 27, 29-32, 63, 65, and 101-102, or allelic variants, truncated variants, or other variants thereof. Such variants are known to one of skill in the art, and include polypeptides having 60%, 70%, 80%, 90%, 91%, 92%, 93%, 94%, 95% or more sequence identity to any of SEQ ID NOS:1, 2, 11, 25, 27, 29-32, 63, 65, and 101-102, or truncated forms thereof. The partially deglycosylated hyaluronidases provided herein also include hybrid, fusion and chimeric partially deglycosylated hyaluronidases, and partially deglycosylated hyaluronidase conjugates.

Glycosidases, or glycoside hydrolases, are enzymes that catalyze the hydrolysis of the glycosidic linkage to generate two smaller sugars. The major types of N-glycans in vertebrates include high mannose glycans, hybrid glycans and complex glycans. There are several glycosidases that result in only partial protein deglycosylation, including: EndoF1, which cleaves high mannose- and hybrid-type glycans; EndoF2, which cleaves biantennary complex-type glycans; EndoF3, which cleaves biantennary and more branched complex glycans; and EndoH, which cleaves high mannose- and hybrid-type glycans. Treatment of a hyaluronan-degrading enzyme, such as a soluble hyaluronidase, such as a soluble PH20, with one or all of these glycosidases can result in only partial deglycosylation and, therefore, retention of hyaluronidase activity.

Partially deglycosylated hyaluronan-degrading enzymes, such as partially deglycosylated soluble hyaluronidases, can be produced by digestion with one or more glycosidases, generally a glycosidase that does not remove all N-glycans but only partially deglycosylates the protein. For example, treatment of PH20 (e.g. a recombinant PH20 designated rHuPH20) with one or all of the above glycosidases (e.g. EndoF1, EndoF2 and/or EndoF3) results in partial deglycosylation. These partially deglycosylated PH20 polypeptides can exhibit hyaluronidase enzymatic activity that is comparable to the fully glycosylated polypeptides. In contrast, treatment of PH20 with PNGaseF, a glycosidase that cleaves all N-glycans, results in complete removal of all N-glycans and thereby renders PH20 enzymatically inactive. Thus, although all N-linked glycosylation sites (such as, for example, those at amino acids N82, N166, N235, N254, N368, and N393 of human PH20, exemplified in SEQ ID NO:1) can be glycosylated, treatment with one or more glycosidases can render the extent of glycosylation reduced compared to a hyaluronidase that is not digested with one or more glycosidases.

The partially deglycosylated hyaluronan-degrading enzymes, including partially deglycosylated soluble PH20 polypeptides, can have 10%, 20%, 30%, 40%, 50%, 60%, 70% or 80% of the level of glycosylation of a fully glycosylated polypeptide. In one example, 1, 2, 3, 4, 5 or 6 of the N-glycosylation sites corresponding to amino acids N82, N166, N235, N254, N368 and N393 of SEQ ID NO:1 are partially deglycosylated, such that they no longer contain high mannose- or complex-type glycans, but rather contain at least an N-acetylglucosamine moiety. In some examples, 1, 2 or 3 of the N-glycosylation sites corresponding to amino acids N82, N166 and N254 of SEQ ID NO:1 are deglycosylated, that is, they do not contain a sugar moiety. In other examples, 3, 4, 5 or 6 of the N-glycosylation sites corresponding to amino acids N82, N166, N235, N254, N368 and N393 of SEQ ID NO:1 are glycosylated. Glycosylated amino acid residues minimally contain an N-acetylglucosamine moiety. Typically, the partially deglycosylated hyaluronan-degrading enzymes, including partially deglycosylated soluble PH20 polypeptides, exhibit hyaluronidase activity that is 10%, 20%, 30%, 40%, 50%, 60%, 70%, 80%, 90%, 100%, 110%, 120%, 130%, 140%, 150%, 200%, 300%, 400%, 500%, 1000% or more of the hyaluronidase activity exhibited by the fully glycosylated polypeptide.

5. Modified (Polymer-Conjugated) Hyaluronan-Degrading Enzymes

Covalent or other stable attachment (conjugation) of polymeric molecules, such as polyethylene glycol (PEGylation moiety (PEG)), to the hyaluronan-degrading enzymes, such as hyaluronidases, impart beneficial properties to the resulting hyaluronan-degrading enzyme-polymer composition. Such properties include improved biocompatibility, extension of protein (and enzymatic activity) half-life in the blood, cells and/or in other tissues within a subject, effective shielding of the protein from proteases and hydrolysis, improved biodistribution, enhanced pharmacokinetics and/or pharmacodynamics, and increased water solubility.

Exemplary polymers that can be conjugated to the hyaluronan-degrading enzyme, such as the hyaluronidase, include natural and synthetic homopolymers, such as polyols (i.e. poly-OH), polyamines (i.e. poly-NH₂) and polycarboxyl acids (i.e. poly-COOH), and further heteropolymers, i.e. polymers containing one or more different coupling groups, for example, hydroxyl groups and amine groups. Examples of suitable polymeric molecules include polymeric molecules selected from among polyalkylene oxides (PAO), such as polyalkylene glycols (PAG), including polyethylene glycols (PEG), methoxypolyethylene glycols (mPEG) and polypropylene glycols, PEG-glycidyl ethers (Epox-PEG), PEG-oxycarbonylimidazole (CDI-PEG) branched polyethylene glycols (PEGs), polyvinyl alcohol (PVA), poly(ethyleneimine) (PEI), linear polyamidoamines, polyacrylamide (PAAm), polydimethylacrylamide (PDAAm), polyvinyl alchol (PVA), polycarboxylates, polyvinylpyrrolidone (PVP), poly-D,L-amino acids, polyethylene-co-maleic acid anhydride, polystyrene-co-maleic acid anhydride, dextrans including carboxymethyl-chitosan, dextrin, dextrans, heparin, homologous albumin, celluloses, including methylcellulose, carboxymethylcellulose, ethylcellulose, hydroxyethylcellulose carboxyethylcellulose and hydroxypropylcellulose, hydrolysates of chitosan, starches such as hydroxyethyl-starches and hydroxypropyl-starches, glycogen, agaroses and derivatives thereof, guar gum, pullulan, inulin, xanthan gum, carrageenan, pectin, alginic acid hydrolysates and bio-polymers.

Typically, the polymers are polyalkylene oxides (PAO), such as polyethylene oxides, such as PEG, typically mPEG, which, in comparison to polysaccharides such as dextran and pullulan, have few reactive groups capable of cross-linking. Typically, the polymers are non-toxic polymeric molecules such as (m)polyethylene glycol (mPEG) which can be covalently conjugated to the hyaluronan-degrading enzyme, such as the hyaluronidase (e.g. to attachment groups on the protein's surface) using relatively simple chemistry.

PEGylation of therapeutics has been reported to increase resistance to proteolysis, increase plasma half-life, and decrease antigenicity and immunogenicity. Examples of PEGylation methodologies are known in the art (see for example, Lu and Felix (1994) Int. J. Peptide Protein Res. 43:127-138; Lu and Felix (1993) Peptide Res. 6:140-146; Felix et al. (1995) Int. J. Peptide Res. 46:253-264; Benhar et al. (1994) J. Biol. Chem. 269:13398-13404; Brumeanu et al. (1995) J. Immunol, 154:3088-3095; see also, Caliceti et al. (2003) Adv. Drug Deliv. Rev. 55(10):1261-1277 and Molineux (2003) Pharmacother. 23(8 Pt 2):3S-8S). PEGylation also can be used in the delivery of nucleic acid molecules in vivo. For example, PEGylation of adenovirus can increase stability and gene transfer (see, e.g. Cheng et al. (2003) Pharm. Res. 20(9):1444-1451).

Suitable polymeric molecules for attachment to the hyaluronan-degrading enzymes, including hyaluronidases, include, but are not limited to, polyethylene glycol (PEG) and PEG derivatives such as methoxy-polyethylene glycols (mPEG), PEG-glycidyl ethers (Epox-PEG), PEG-oxycarbonylimidazole (CDI-PEG), branched PEGs, and polyethylene oxide (PEO) (see, e.g. Roberts et al. (2002) Adv. Drug Deliv. Rev. 54:459-476; Harris and Zalipsky (eds.) “Poly(ethylene glycol), Chemistry and Biological Applications” ACS Symposium Series 680 (1997); Mehvar et al. (2000) J. Pharm. Pharmaceut. Sci. 3(1):125-136; Harris (2003) Nature Rev. Drug Discov. 2:214-221; and Tsubery (2004) J. Biol. Chem. 279(37):38118-24). The polymeric molecule can be of a molecular weight typically ranging from about 3 kDa to about 60 kDa. In some embodiments the polymeric molecule that is conjugated to a protein, such as a hyaluronidase, for example a PH20, has a molecular weight of between or about between 5 to 60 kDa, such as at least or about at least or 5, 10, 15, 20, 25, 30, 35, 40, 45, 50, 55, 60 or more than 60 kDa.

PEGylated Soluble Hyaluronan-Degrading Enzymes

The polymer-conjugated hyaluronan-degrading enzyme used in the methods herein can be a PEGylated hyaluronan-degrading enzyme, such as a PEGylated soluble hyaluronan-degrading enzyme. In one example, it is a PEGylated soluble hyaluronidase, e.g. PEGylated PH20. Various methods of modifying polypeptides by covalently attaching (conjugating) a PEG or PEG derivative (i.e. “PEGylation”) are known in the art (see, e.g. U.S. Pub. Nos. 2006/0104968 and 2004/0235734; U.S. Pat. Nos. 5,672,662 and 6,737,505). Techniques for PEGylation include, but are not limited to, specialized linkers and coupling chemistries (see, e.g. Roberts et al. (2002) Adv. Drug Deliv. Rev. 54:459-476), attachment of multiple PEG moieties to a single conjugation site (such as via use of branched PEGs; see, e.g. Guiotto et al. (2002) Bioorg. Med. Chem. Lett. 12:177-180), site-specific PEGylation and/or mono-PEGylation (see, e.g. Chapman et al. (1999) Nature Biotech. 17:780-783), and site-directed enzymatic PEGylation (see, e.g. Sato (2002) Adv. Drug Deliv. Rev. 54:487-504). Methods and techniques described in the art can produce proteins having 1, 2, 3, 4, 5, 6, 7, 8, 9, 10 or more than 10 PEG or PEG derivatives attached to a single protein molecule (see, e.g. U.S. Pub. No. 2006/0104968).

Numerous reagents for PEGylation have been described in the art. Such reagents include, but are not limited to, N-hydroxysuccinimidyl (NHS) activated PEG, succinimidyl mPEG, mPEG₂-N-hydroxysuccinimide, mPEG succinimidyl alpha-methylbutanoate, mPEG succinimidyl propionate, mPEG succinimidyl butanoate, mPEG carboxymethyl 3-hydroxybutanoic acid succinimidyl ester, homobifunctional PEG-succinimidyl propionate, homobifunctional PEG propionaldehyde, homobifunctional PEG butyraldehyde, PEG maleimide, PEG hydrazide, p-nitrophenyl-carbonate PEG, mPEG-benzotriazole carbonate, propionaldehyde PEG, mPEG butryaldehyde, branched mPEG₂ butyraldehyde, mPEG acetyl, mPEG piperidone, mPEG methylketone, mPEG “linkerless” maleimide, mPEG vinyl sulfone, mPEG thiol, mPEG orthopyridylthioester, mPEG orthopyridyl disulfide, Fmoc-PEG-NHS, Boc-PEG-NHS, vinylsulfone PEG-NHS, acrylate PEG-NHS, fluorescein PEG-NHS, and biotin PEG-NHS (see, e.g. Monfardini et al. (1995) Bioconj. Chem. 6:62-69; Veronese et al. (1997) J. Bioactive Compatible Polymers 12:197-207; U.S. Pat. No. 5,672,662; U.S. Pat. No. 5,932,462; U.S. Pat. No. 6,495,659; U.S. Pat. No. 6,737,505; U.S. Pat. No. 4,002,531; U.S. Pat. No. 4,179,337; U.S. Pat. No. 5,122,614; U.S. Pat. No. 5,324,844; U.S. Pat. No. 5,446,090; U.S. Pat. No. 5,612,460; U.S. Pat. No. 5,643,575; U.S. Pat. No. 5,766,581; U.S. Pat. No. 5,795,569; U.S. Pat. No. 5,808,096; U.S. Pat. No. 5,900,461; U.S. Pat. No. 5,919,455; U.S. Pat. No. 5,985,263; U.S. Pat. No. 5,990,237; U.S. Pat. No. 6,113,906; U.S. Pat. No. 6,214,966; U.S. Pat. No. 6,258,351; U.S. Pat. No. 6,340,742; U.S. Pat. No. 6,413,507; U.S. Pat. No. 6,420,339; U.S. Pat. No. 6,437,025; U.S. Pat. No. 6,448,369; U.S. Pat. No. 6,461,802; U.S. Pat. No. 6,828,401; U.S. Pat. No. 6,858,736; U.S. Pub. Nos. 2001/0021763; U.S. 2001/0044526; U.S. 2001/0046481; U.S. 2002/0052430; U.S. 2002/0072573; U.S. 2002/0156047; U.S. 2003/0114647; U.S. 2003/0143596; U.S. 2003/0158333; U.S. 2003/0220447; U.S. 2004/0013637; US 2004/0235734; U.S. 2005/0114037; U.S. 2005/0171328; U.S. 2005/0209416; International Pat. App. Nos. WO 2005/00360; WO 01/076640; WO 00/02017; WO 02/49673; WO 94/28024; WO 01/87925; and EP Pat. Nos. 1064951 and EP 0822199).

### D. METHODS OF PRODUCING NUCLEIC ACIDS AND ENCODED POLYPEPTIDES OF HYALURONAN-DEGRADING ENZYMES

Polypeptides of a hyaluronan-degrading enzyme, such as a soluble hyaluronidase, set forth herein, can be obtained by methods well known in the art for protein purification and recombinant protein expression. Any method known to those of skill in the art for identification of nucleic acids that encode desired genes can be used. Any method available in the art can be used to obtain a full length (i.e. encompassing the entire coding region) cDNA or genomic DNA clone encoding a hyaluronidase, such as from a cell or tissue source. Modified or variant soluble hyaluronidases can be engineered from a wildtype polypeptide, such as by site-directed mutagenesis.

Polypeptides can be cloned or isolated using any available methods known in the art for cloning and isolating nucleic acid molecules. Such methods include PCR amplification of nucleic acids and screening of libraries, including nucleic acid hybridization screening, antibody-based screening and activity-based screening.

Methods for amplification of nucleic acids can be used to isolate nucleic acid molecules encoding a desired polypeptide, including for example, polymerase chain reaction (PCR) methods. A nucleic acid-containing material can be used as a starting material from which a desired polypeptide-encoding nucleic acid molecule can be isolated. For example, DNA and mRNA preparations, cell extracts, tissue extracts, fluid samples (e.g. blood, serum, saliva), samples from healthy and/or diseased subjects can be used in amplification methods. Nucleic acid libraries also can be used as a source of starting material. Primers can be designed to amplify a desired polypeptide. For example, primers can be designed based on expressed sequences from which a desired polypeptide is generated. Primers can be designed based on back-translation of a polypeptide amino acid sequence. Nucleic acid molecules generated by amplification can be sequenced and confirmed to encode a desired polypeptide.

Additional nucleotide sequences can be joined to a polypeptide-encoding nucleic acid molecule, including linker sequences containing restriction endonuclease sites for the purpose of cloning the synthetic gene into a vector, for example, a protein expression vector or a vector designed for the amplification of the core protein coding DNA sequences. Furthermore, additional nucleotide sequences specifying functional DNA elements can be operatively linked to a polypeptide-encoding nucleic acid molecule. Examples of such sequences include, but are not limited to, promoter sequences designed to facilitate intracellular protein expression, and secretion sequences, for example heterologous signal sequences, designed to facilitate protein secretion. Such sequences are known to those of skill in the art. Additional nucleotide residue sequences such as sequences of bases specifying protein-binding regions also can be linked to enzyme-encoding nucleic acid molecules. Such regions include, but are not limited to, sequences of residues that facilitate or encode proteins that facilitate uptake of an enzyme into specific target cells, or otherwise alter pharmacokinetics of a product of a synthetic gene. For example, enzymes can be linked to PEG moieties.

In addition, tags or other moieties can be added, for example, to aid in detection or affinity purification of the polypeptide. For example, additional nucleotide residues sequences such as sequences of bases specifying an epitope tag or other detectable marker also can be linked to enzyme-encoding nucleic acid molecules. Exemplary of such sequences include nucleic acid sequences encoding a His tag (e.g. 6×His, HHHHHH; SEQ ID NO:54) or Flag Tag (DYKDDDDK; SEQ ID NO:55).

The identified and isolated nucleic acids can then be inserted into an appropriate cloning vector. A large number of vector-host systems known in the art can be used. Possible vectors include, but are not limited to, plasmids or modified viruses, but the vector system must be compatible with the host cell used. Such vectors include, but are not limited to, bacteriophages such as lambda derivatives, or plasmids such as pCMV4, pBR322 or pUC plasmid derivatives, or the Bluescript vector (Stratagene, La Jolla, Calif.). Other expression vectors include the HZ24 expression vector exemplified herein. The insertion into a cloning vector can, for example, be accomplished by ligating the DNA fragment into a cloning vector which has complementary cohesive termini. Insertion can be effected using TOPO cloning vectors (Invitrogen, Carlsbad, Calif.). If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized oligonucleotides encoding restriction endonuclease recognition sequences. In an alternative method, the cleaved vector and protein gene can be modified by homopolymeric tailing. Recombinant molecules can be introduced into host cells via, for example, transformation, transfection, infection, electroporation and sonoporation, so that many copies of the gene sequence are generated.

In specific embodiments, transformation of host cells with recombinant DNA molecules that incorporate the isolated protein gene, cDNA, or synthesized DNA sequence enables generation of multiple copies of the gene. Thus, the gene can be obtained in large quantities by growing transformants, isolating the recombinant DNA molecules from the transformants and, when necessary, retrieving the inserted gene from the isolated recombinant DNA.

1. Vectors and Cells

For recombinant expression of one or more of the desired proteins, such as any hyaluronan-degrading enzyme polypeptide described herein, the nucleic acid containing all or a portion of the nucleotide sequence encoding the protein can be inserted into an appropriate expression vector, i.e. a vector that contains the necessary elements for the transcription and translation of the inserted protein coding sequence. The necessary transcriptional and translational signals also can be supplied by the native promoter for enzyme genes, and/or their flanking regions.

Also provided are vectors that contain a nucleic acid encoding the enzyme. Cells containing the vectors also are provided. The cells include eukaryotic and prokaryotic cells, and the vectors are any suitable for use therein.

Prokaryotic and eukaryotic cells, including endothelial cells, containing the vectors are provided. Such cells include bacterial cells, yeast cells, fungal cells, Archea, plant cells, insect cells and animal cells. The cells are used to produce a protein thereof by growing the above-described cells under conditions whereby the encoded protein is expressed by the cell, and recovering the expressed protein. For purposes herein, for example, the enzyme can be secreted into the medium.

Provided are vectors that contain a sequence of nucleotides that encodes the hyaluronan-degrading enzyme polypeptide, in some examples a soluble hyaluronidase polypeptide, coupled to the native or heterologous signal sequence, as well as multiple copies thereof. The vectors can be selected for expression of the enzyme protein in the cell or such that the enzyme protein is expressed as a secreted protein.

A variety of host-vector systems can be used to express the protein coding sequence. These include but are not limited to mammalian cell systems infected with virus (e.g. vaccinia virus, adenovirus and other viruses); insect cell systems infected with virus (e.g. baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system used, any one of a number of suitable transcription and translation elements can be used.

Any methods known to those of skill in the art for the insertion of DNA fragments into a vector can be used to construct expression vectors containing a chimeric gene containing appropriate transcriptional/translational control signals and protein coding sequences. These methods can include in vitro recombinant DNA and synthetic techniques and in vivo recombinants (genetic recombination). Expression of nucleic acid sequences encoding protein, or domains, derivatives, fragments or homologs thereof, can be regulated by a second nucleic acid sequence so that the genes or fragments thereof are expressed in a host transformed with the recombinant DNA molecule(s). For example, expression of the proteins can be controlled by any promoter/enhancer known in the art. In a specific embodiment, the promoter is not native to the genes for a desired protein. Promoters which can be used include, but are not limited to, the SV40 early promoter (Bernoist and Chambon (1981) Nature 290:304-310), the promoter contained in the 3′ long terminal repeat of Rous sarcoma virus (Yamamoto et al. (1980) Cell 22:787-797), the herpes thymidine kinase promoter (Wagner et al. (1981) Proc. Natl. Acad. Sci. U.S.A. 78:1441-1445), the regulatory sequences of the metallothionein gene (Brinster et al. (19982) Nature 296:39-42); prokaryotic expression vectors such as the β-lactamase promoter (Jay et al. (1981) Proc. Natl. Acad. Sci. U.S.A. 78:5543) or the tac promoter (DeBoer et al. (1983) Proc. Natl. Acad. Sci. U.S.A. 80:21-25; see also, “Useful Proteins from Recombinant Bacteria” in Sci. Am. (1980) 242:79-94); plant expression vectors containing the nopaline synthetase promoter (Herrara-Estrella et al. (1984) Nature 303:209-213) or the cauliflower mosaic virus 35S RNA promoter (Gardner et al. (1981) Nucleic Acids Res. 9:2871), and the promoter of the photosynthetic enzyme ribulose bisphosphate carboxylase (Herrera-Estrella et al. (1984) Nature 310:115-120); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase 1 gene control region which is active in pancreatic acinar cells (Swift et al. (1984) Cell 38:639-646; Ornitz et al. (1986) Cold Spring Harbor Symp. Quant. Biol. 50:399-409; MacDonald (1987) Hepatology 7:425-515); insulin gene control region which is active in pancreatic beta cells (Hanahan et al. (1985) Nature 315:115-122), immunoglobulin gene control region which is active in lymphoid cells (Grosschedl et al. (1984) Cell 38:647-658; Adams et al. (1985) Nature 318:533-538; Alexander et al. (1987) Mol. Cell. Biol. 7:1436-1444), mouse mammary tumor virus control region which is active in testicular, breast, lymphoid and mast cells (Leder et al. (1986) Cell 45:485-495), albumin gene control region which is active in liver (Pinkert et al. (1987) Genes Dev. 1:268-276), alpha-fetoprotein gene control region which is active in liver (Krumlauf et al. (1985) Mol. Cell. Biol. 5:1639-1648; Hammer et al. (1987) Science 235:53-58), alpha-1 antitrypsin gene control region which is active in liver (Kelsey et al. (1987) Genes Dev. 1:161-171), beta globin gene control region which is active in myeloid cells (Magram et al. (1985) Nature 315:338-340; Kollias et al. (1986) Cell 46:89-94), myelin basic protein gene control region which is active in oligodendrocyte cells of the brain (Readhead et al. (1987) Cell 48:703-712), myosin light chain-2 gene control region which is active in skeletal muscle (Shani (1985) Nature 314:283-286), and gonadotrophic releasing hormone gene control region which is active in gonadotrophs of the hypothalamus (Mason et al. (1986) Science 234:1372-1378).

In a specific embodiment, a vector is used that contains a promoter operably linked to nucleic acids encoding a desired protein, or a domain, fragment, derivative or homolog, thereof, one or more origins of replication, and optionally, one or more selectable markers (e.g. an antibiotic resistance gene). Exemplary plasmid vectors for transformation of E. coli cells, include, for example, the pQE expression vectors (available from Qiagen, Valencia, Calif.; see also literature published by Qiagen describing the system). pQE vectors have a phage T5 promoter (recognized by E. coli RNA polymerase) and a double lac operator repression module to provide tightly regulated, high-level expression of recombinant proteins in E. coli, a synthetic ribosomal binding site (RBS II) for efficient translation, a 6×His tag coding sequence, t₀ and T1 transcriptional terminators, ColE1 origin of replication, and a beta-lactamase gene for conferring ampicillin resistance. The pQE vectors enable placement of a 6×His tag at either the N- or C-terminus of the recombinant protein. Such plasmids include pQE 32, pQE 30, and pQE 31 which provide multiple cloning sites for all three reading frames and provide for the expression of N-terminally 6×His-tagged proteins. Other exemplary plasmid vectors for transformation of E. coli cells, include, for example, the pET expression vectors (see, U.S. Pat. No. 4,952,496; available from Novagen, Madison, Wis.; see also, literature published by Novagen describing the system). Such plasmids include pET 11a, which contains the T7lac promoter, T7 terminator, the inducible E. coli lac operator, and the lac repressor gene; pET 12a-c, which contains the T7 promoter, T7 terminator, and the E. coli ompT secretion signal; and pET 15b and pET19b (Novagen, Madison, Wis.), which contain a His-Tag™ leader sequence for use in purification with a His column and a thrombin cleavage site that permits cleavage following purification over the column, the T7-lac promoter region and the T7 terminator.

Exemplary of a vector for mammalian cell expression is the HZ24 expression vector. The HZ24 expression vector was derived from the pCI vector backbone (Promega). It contains DNA encoding the Beta-lactamase resistance gene (AmpR), an F1 origin of replication, a Cytomegalovirus immediate-early enhancer/promoter region (CMV), and an SV40 late polyadenylation signal (SV40). The expression vector also has an internal ribosome entry site (IRES) from the ECMV virus (Clontech) and the mouse dihydrofolate reductase (DHFR) gene.

2. Expression

Hyaluronan-degrading enzyme polypeptides, including soluble hyaluronidase polypeptides, can be produced by any method known to those of skill in the art including in vivo and in vitro methods. Desired proteins can be expressed in any organism suitable to produce the required amounts and forms of the proteins, such as for example, needed for administration and treatment. Expression hosts include prokaryotic and eukaryotic organisms such as E. coli, yeast, plants, insect cells, mammalian cells, including human cell lines and transgenic animals. Expression hosts can differ in their protein production levels as well as the types of post-translational modifications that are present on the expressed proteins. The choice of expression host can be made based on these and other factors, such as regulatory and safety considerations, production costs and the need and methods for purification.

Many expression vectors are available and known to those of skill in the art and can be used for expression of proteins. The choice of expression vector will be influenced by the choice of host expression system. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector.

Hyaluronan-degrading enzyme polypeptides, such as soluble hyaluronidase polypeptides, also can be utilized or expressed as protein fusions. For example, an enzyme fusion can be generated to add additional functionality to an enzyme. Examples of enzyme fusion proteins include, but are not limited to, fusions of a signal sequence, a tag such as for localization, e.g. a his₆ tag or a myc tag, or a tag for purification, for example, a GST fusion, and a sequence for directing protein secretion and/or membrane association.

a. Prokaryotic Cells

Prokaryotes, especially E. coli, provide a system for producing large amounts of proteins. Transformation of E. coli is a simple and rapid technique well known to those of skill in the art. Expression vectors for E. coli can contain inducible promoters, such promoters are useful for inducing high levels of protein expression and for expressing proteins that exhibit some toxicity to the host cells. Examples of inducible promoters include the lac promoter, the trp promoter, the hybrid tac promoter, the T7 and SP6 RNA promoters and the temperature regulated XPL promoter.

Proteins, such as any provided herein, can be expressed in the cytoplasmic environment of E. coli. The cytoplasm is a reducing environment and for some molecules, this can result in the formation of insoluble inclusion bodies. Reducing agents such as dithiothreitol and β-mercaptoethanol and denaturants, such as guanidine-HCl and urea can be used to resolubilize the proteins. An alternative approach is the expression of proteins in the periplasmic space of bacteria which provides an oxidizing environment and chaperonin-like and disulfide isomerases and can lead to the production of soluble protein. Typically, a leader sequence is fused to the protein to be expressed which directs the protein to the periplasm. The leader is then removed by signal peptidases inside the periplasm. Examples of periplasmic-targeting leader sequences include the pelB leader from the pectate lyase gene and the leader derived from the alkaline phosphatase gene. In some cases, periplasmic expression allows leakage of the expressed protein into the culture medium. The secretion of proteins allows quick and simple purification from the culture supernatant. Proteins that are not secreted can be obtained from the periplasm by osmotic lysis. Similar to cytoplasmic expression, in some cases proteins can become insoluble and denaturants and reducing agents can be used to facilitate solubilization and refolding. Temperature of induction and growth also can influence expression levels and solubility, typically temperatures between 25° C. and 37° C. are used. Typically, bacteria produce aglycosylated proteins. Thus, if proteins require glycosylation for function, glycosylation can be added in vitro after purification from host cells.

b. Yeast Cells

Yeasts such as Saccharomyces cerevisae, Schizosaccharomyces pombe, Yarrowia lipolytica. Kluyveromyces laths and Pichia pastoris are well known yeast expression hosts that can be used for production of proteins, such as any described herein. Yeast can be transformed with episomal replicating vectors or by stable chromosomal integration by homologous recombination. Typically, inducible promoters are used to regulate gene expression. Examples of such promoters include GAL1, GAL7 and GALS and metallothionein promoters, such as CUP 1, AOX 1 or other Pichia or other yeast promoter. Expression vectors often include a selectable marker such as LEU2, TRP1, HIS3 and URA3 for selection and maintenance of the transformed DNA. Proteins expressed in yeast are often soluble. Co-expression with chaperonins such as Bip and protein disulfide isomerase can improve expression levels and solubility. Additionally, proteins expressed in yeast can be directed for secretion using secretion signal peptide fusions such as the yeast mating type alpha-factor secretion signal from Saccharomyces cerevisae and fusions with yeast cell surface proteins such as the Aga2p mating adhesion receptor or the Arxula adeninivorans glucoamylase. A protease cleavage site such as for the Kex-2 protease, can be engineered to remove the fused sequences from the expressed polypeptides as they exit the secretion pathway. Yeast also is capable of glycosylation at Asn-X-Ser/Thr motifs.

c. Insect Cells

Insect cells, particularly using baculovirus expression, are useful for expressing polypeptides such as hyaluronidase polypeptides. Insect cells express high levels of protein and are capable of most of the post-translational modifications used by higher eukaryotes. Baculovirus have a restrictive host range which improves the safety and reduces regulatory concerns of eukaryotic expression. Typical expression vectors use a promoter for high level expression such as the polyhedrin promoter of baculovirus. Commonly used baculovirus systems include the baculoviruses such as Autographa califormica nuclear polyhedrosis virus (AcNPV), and the Bombyx mori nuclear polyhedrosis virus (BmNPV) and an insect cell line such as Sf9 derived from Spodoptera frupperda, Pseudaletia unipuncta (A7S) and Danaus plexippus (DpN1). For high-level expression, the nucleotide sequence of the molecule to be expressed is fused immediately downstream of the polyhedrin initiation codon of the virus. Mammalian secretion signals are accurately processed in insect cells and can be used to secrete the expressed protein into the culture medium. In addition, the cell lines Pseudaletia unipuncta (A7S) and Danaus plexippus (DpN1) produce proteins with glycosylation patterns similar to mammalian cell systems.

An alternative expression system in insect cells is the use of stably transformed cells. Cell lines such as the Schneider 2 (S2) and Kc cells (Drosophila melanogaster) and C7 cells (Aedes albopictus) can be used for expression. The Drosophila metallothionein promoter can be used to induce high levels of expression in the presence of heavy metal induction with cadmium or copper. Expression vectors are typically maintained by the use of selectable markers such as neomycin and hygromycin.

d. Mammalian Cells

Mammalian expression systems can be used to express proteins including hyaluronan-degrading enzyme polypeptides, such as soluble hyaluronidase polypeptides. Expression constructs can be transferred to mammalian cells by viral infection such as adenovirus or by direct DNA transfer such as liposomes, calcium phosphate, DEAE-dextran and by physical means such as electroporation and microinjection. Expression vectors for mammalian cells typically include an mRNA cap site, a TATA box, a translational initiation sequence (Kozak consensus sequence) and polyadenylation elements. IRES elements also can be added to permit bicistronic expression with another gene, such as a selectable marker. Such vectors often include transcriptional promoter-enhancers for high-level expression, for example the SV40 promoter-enhancer, the human cytomegalovirus (CMV) promoter and the long terminal repeat of Rous sarcoma virus (RSV). These promoter-enhancers are active in many cell types. Tissue and cell-type promoters and enhancer regions also can be used for expression. Exemplary promoter/enhancer regions include, but are not limited to, those from genes such as elastase I, insulin, immunoglobulin, mouse mammary tumor virus, albumin, alpha fetoprotein, alpha I antitrypsin, beta globin, myelin basic protein, myosin light chain 2, and gonadotropic releasing hormone gene control. Selectable markers can be used to select for and maintain cells with the expression construct. Examples of selectable marker genes include, but are not limited to, hygromycin B phosphotransferase, adenosine deaminase, xanthine-guanine phosphoribosyl transferase, aminoglycoside phosphotransferase, dihydrofolate reductase (DHFR) and thymidine kinase. For example, expression can be performed in the presence of methotrexate to select for only those cells expressing the DHFR gene. Fusion with cell surface signaling molecules such as TCR-ζ and Fc_{ε}RI-γ can direct expression of the proteins in an active state on the cell surface.

Many cell lines are available for mammalian expression including mouse, rat human, monkey, chicken and hamster cells. Exemplary cell lines include but are not limited to CHO, Balb/3T3, HeLa, MT2, mouse NSO (nonsecreting) and other myeloma cell lines, hybridoma and heterohybridoma cell lines, lymphocytes, fibroblasts, Sp2/0, COS, NIH3T3, HEK293, 293S, 2B8, and HKB cells. Cell lines also are available adapted to serum-free media which facilitates purification of secreted proteins from the cell culture media. Examples include CHO-S cells (Invitrogen, Carlsbad, Calif., cat #11619-012) and the serum free EBNA-1 cell line (Pham et al. (2003) Biotechnol. Bioeng. 84:332-342). Cell lines also are available that are adapted to grow in special media optimized for maximal expression. For example, DG44 CHO cells are adapted to grow in suspension culture in a chemically defined, animal product-free medium.

e. Plants

Transgenic plant cells and plants can be used to express proteins such as any described herein. Expression constructs are typically transferred to plants using direct DNA transfer such as microprojectile bombardment and PEG-mediated transfer into protoplasts, and with agrobacterium-mediated transformation. Expression vectors can include promoter and enhancer sequences, transcriptional termination elements and translational control elements. Expression vectors and transformation techniques are usually divided between dicot hosts, such as Arabidopsis and tobacco, and monocot hosts, such as corn and rice. Examples of plant promoters used for expression include the cauliflower mosaic virus promoter, the nopaline synthetase promoter, the ribose bisphosphate carboxylase promoter and the ubiquitin and UBQ3 promoters. Selectable markers such as hygromycin, phosphomannose isomerase and neomycin phosphotransferase are often used to facilitate selection and maintenance of transformed cells. Transformed plant cells can be maintained in culture as cells, aggregates (callus tissue) or regenerated into whole plants. Transgenic plant cells also can include algae engineered to produce hyaluronidase polypeptides. Because plants have different glycosylation patterns than mammalian cells, this can influence the choice of protein produced in these hosts.

3. Purification Techniques

Method for purification of polypeptides, including hyaluronan-degrading enzyme polypeptides (e.g. soluble hyaluronidase polypeptides) or other proteins, from host cells will depend on the chosen host cells and expression systems. For secreted molecules, proteins are generally purified from the culture media after removing the cells. For intracellular expression, cells can be lysed and the proteins purified from the extract. When transgenic organisms such as transgenic plants and animals are used for expression, tissues or organs can be used as starting material to make a lysed cell extract. Additionally, transgenic animal production can include the production of polypeptides in milk or eggs, which can be collected, and if necessary, the proteins can be extracted and further purified using standard methods in the art.

Proteins, such as soluble hyaluronidase polypeptides, can be purified using standard protein purification techniques known in the art including but not limited to, SDS-PAGE, size fraction and size exclusion chromatography, ammonium sulfate precipitation and ionic exchange chromatography, such as anion exchange chromatography. Affinity purification techniques also can be utilized to improve the efficiency and purity of the preparations. For example, antibodies, receptors and other molecules that bind hyaluronidase enzymes can be used in affinity purification. Expression constructs also can be engineered to add an affinity tag to a protein such as a myc epitope, GST fusion or His₆ and affinity purified with myc antibody, glutathione resin and Ni-resin, respectively. Purity can be assessed by any method known in the art including gel electrophoresis and staining and spectrophotometric techniques. Purified rHuPH20 compositions, as provided herein, typically have a specific activity of at least 70,000 to 100,000 Units/mg, for example, about 120,000 Units/mg. The specific activity can vary upon modification, such as with a polymer. For example, polymer-conjugated hyaluronan-degrading enzymes typically have a specific activity of between or between about 15,000 to 60,000 Units/mg, such as for example, 30,000 Units/mg to 50,000 Units/mg, and generally at least or about at least 40,000 Units/mg.

4. PEGylation of Hyaluronan-Degrading Enzyme Polypeptides

Polyethylene glycol (PEG) has been widely used in biomaterials, biotechnology and medicine primarily because PEG is a biocompatible, nontoxic, water-soluble polymer that is typically nonimmunogenic (Zhao and Harris (1997) ACS Symposium Series 680:458-472). In the area of drug delivery, PEG derivatives have been widely used in covalent attachment (i.e. “PEGylation”) to proteins to reduce immunogenicity, proteolysis and kidney clearance and to enhance solubility (Zalipsky (1995) Adv. Drug Del. Rev. 16:157-182). Similarly, PEG has been attached to low molecular weight, relatively hydrophobic drugs to enhance solubility, reduce toxicity and alter biodistribution. Typically, PEGylated drugs are injected as solutions.

A closely related application is synthesis of crosslinked degradable PEG networks or formulations for use in drug delivery since much of the same chemistry used in design of degradable, soluble drug carriers can also be used in design of degradable gels (Sawhney et al. (1993) Macromolecules 26:581-587). It also is known that intermacromolecular complexes can be formed by mixing solutions of two complementary polymers. Such complexes are generally stabilized by electrostatic interactions (polyanion-polycation) and/or hydrogen bonds (polyacid-polybase) between the polymers involved, and/or by hydrophobic interactions between the polymers in an aqueous surrounding (Krupers et al. (1996) Eur. Polym. J. 32:785-790). For example, mixing solutions of polyacrylic acid (PAAc) and polyethylene oxide (PEO) under the proper conditions results in the formation of complexes based mostly on hydrogen bonding. Dissociation of these complexes at physiologic conditions has been used for delivery of free drugs (i.e. non-PEGylated). In addition, complexes of complementary polymers have been formed from both homopolymers and copolymers.

Numerous reagents for PEGylation have been described in the art. Such reagents include, but are not limited to, N-hydroxysuccinimidyl (NHS) activated PEG, succinimidyl mPEG, mPEG₂-N-hydroxysuccinimide, mPEG succinimidyl alpha-methylbutanoate, mPEG succinimidyl propionate, mPEG succinimidyl butanoate, mPEG carboxymethyl 3-hydroxybutanoic acid succinimidyl ester, homobi functional PEG-succinimidyl propionate, homobi functional PEG propionaldehyde, homobi functional PEG butyraldehyde, PEG maleimide, PEG hydrazide, p-nitrophenyl-carbonate PEG, mPEG-benzotriazole carbonate, propionaldehyde PEG, mPEG butryaldehyde, branched mPEG₂ butyraldehyde, mPEG acetyl, mPEG piperidone, mPEG methylketone, mPEG “linkerless” maleimide, mPEG vinyl sulfone, mPEG thiol, mPEG orthopyridylthioester, mPEG orthopyridyl disulfide, Fmoc-PEG-NHS, Boc-PEG-NHS, vinylsulfone PEG-NHS, acrylate PEG-NHS, fluorescein PEG-NHS, and biotin PEG-NHS (see, e.g. Monfardini et al. (1995) Bioconj. Chem. 6:62-69; Veronese et al. (1997) J. Bioactive Compatible Polymers 12:197-207; U.S. Pat. No. 5,672,662; U.S. Pat. No. 5,932,462; U.S. Pat. No. 6,495,659; U.S. Pat. No. 6,737,505; U.S. Pat. No. 4,002,531; U.S. Pat. No. 4,179,337; U.S. Pat. No. 5,122,614; U.S. Pat. No. 5,324,844; U.S. Pat. No. 5,446,090; U.S. Pat. No. 5,612,460; U.S. Pat. No. 5,643,575; U.S. Pat. No. 5,766,581; U.S. Pat. No. 5,795,569; U.S. Pat. No. 5,808,096; U.S. Pat. No. 5,900,461; U.S. Pat. No. 5,919,455; U.S. Pat. No. 5,985,263; U.S. Pat. No. 5,990,237; U.S. Pat. No. 6,113,906; U.S. Pat. No. 6,214,966; U.S. Pat. No. 6,258,351; U.S. Pat. No. 6,340,742; U.S. Pat. No. 6,413,507; U.S. Pat. No. 6,420,339; U.S. Pat. No. 6,437,025; U.S. Pat. No. 6,448,369; U.S. Pat. No. 6,461,802; U.S. Pat. No. 6,828,401; U.S. Pat. No. 6,858,736; U.S. 2001/0021763; U.S. 2001/0044526; U.S. 2001/0046481; U.S. 2002/0052430; U.S. 2002/0072573; U.S. 2002/0156047; U.S. 2003/0114647; U.S. 2003/0143596; U.S. 2003/0158333; U.S. 2003/0220447; U.S. 2004/0013637; US 2004/0235734; WO0500360; U.S. 2005/0114037; U.S. 2005/0171328; U.S. 2005/0209416; EP 1064951; EP 0822199; WO 01076640; WO 0002017; WO 0249673; WO 9428024; and WO 0187925).

In one example, the polyethylene glycol has a molecular weight ranging from about 3 kD to about 50 kD, and typically from about 5 kD to about 30 kD. Covalent attachment of the PEG to the drug (known as “PEGylation”) can be accomplished by known chemical synthesis techniques. For example, the PEGylation of protein can be accomplished by reacting NHS-activated PEG with the protein under suitable reaction conditions.

While numerous reactions have been described for PEGylation, those that are most generally applicable confer directionality, utilize mild reaction conditions, and do not necessitate extensive downstream processing to remove toxic catalysts or bi-products. For instance, monomethoxy PEG (mPEG) has only one reactive terminal hydroxyl, and thus its use limits some of the heterogeneity of the resulting PEG-protein product mixture. Activation of the hydroxyl group at the end of the polymer opposite to the terminal methoxy group is generally necessary to accomplish efficient protein PEGylation, with the aim being to make the derivatised PEG more susceptible to nucleophilic attack. The attacking nucleophile is usually the epsilon-amino group of a lysyl residue, but other amines also can react (e.g. the N-terminal alpha-amine or the ring amines of histidine) if local conditions are favorable. A more directed attachment is possible in proteins containing a single lysine or cysteine. The latter residue can be targeted by PEG-maleimide for thiol-specific modification. Alternatively, PEG hydrazide can be reacted with a periodate oxidized hyaluronan-degrading enzyme and reduced in the presence of NaCNBH₃. More specifically, PEGylated CMP sugars can be reacted with a hyaluronan-degrading enzyme in the presence of appropriate glycosyl-transferases. One technique is the “PEGylation” technique where a number of polymeric molecules are coupled to the polypeptide in question. When using this technique the immune system has difficulties in recognizing the epitopes on the polypeptide's surface responsible for the formation of antibodies, thereby reducing the immune response. For polypeptides introduced directly into the circulatory system of the human body to give a particular physiological effect (i.e. pharmaceuticals) the typical potential immune response is an IgG and/or IgM response, while polypeptides which are inhaled through the respiratory system (i.e. industrial polypeptide) potentially can cause an IgE response (i.e. allergic response). One of the theories explaining the reduced immune response is that the polymeric molecule(s) shield(s) epitope(s) on the surface of the polypeptide responsible for the immune response leading to antibody formation. Another theory or at least a partial factor is that the heavier the conjugate is, the more reduced immune response is obtained.

Typically, to make the PEGylated hyaluronan-degrading enzymes provided herein, including the PEGylated hyaluronidases, PEG moieties are conjugated, via covalent attachment, to the polypeptides. Techniques for PEGylation include, but are not limited to, specialized linkers and coupling chemistries (see, e.g. Roberts et al. (2002) Adv. Drug Deliv. Rev. 54:459-476), attachment of multiple PEG moieties to a single conjugation site (such as via use of branched PEGs; see, e.g. Guiotto et al. (2002) Bioorg. Med. Chem. Lett. 12:177-180), site-specific PEGylation and/or mono-PEGylation (see, e.g. Chapman et al. (1999) Nature Biotech. 17:780-783), and site-directed enzymatic PEGylation (see, e.g. Sato (2002) Adv. Drug Deliv. Rev. 54:487-504). Methods and techniques described in the art can produce proteins having 1, 2, 3, 4, 5, 6, 7, 8, 9, 10 or more than 10 PEG or PEG derivatives attached to a single protein molecule (see, e.g. U.S. Pub. No. 2006/0104968).

As an exemplary illustration of the PEGylation of an illustrative method for making PEGylated hyaluronan-degrading enzymes, such as PEGylated hyaluronidases, PEG aldehydes, succinimides and carbonates have each been applied to conjugate PEG moieties, typically succinimidyl PEGs, to rHuPH20. For example, rHuPH20 has been conjugated with exemplary succinimidyl monoPEG (mPEG) reagents including mPEG-Succinimidyl Propionates (mPEG-SPA), mPEG-Succinimidyl Butanoates (mPEG-SBA), and (for attaching “branched” PEGs) mPEG2-N-Hydroxylsuccinimide. These PEGylated succinimidyl esters contain different length carbon backbones between the PEG group and the activated cross-linker, and either a single or branched PEG group. These differences can be used, for example, to provide for different reaction kinetics and to potentially restrict sites available for PEG attachment to rHuPH20 during the conjugation process.

Succinimidyl PEGs (as above) containing either linear or branched PEGs can be conjugated to rHuPH20. PEGs can used to generate rHuPH20s reproducibly containing molecules having, on the average, between about three to six or three to six PEG molecules per hyaluronidase. Such PEGylated rHuPH20 compositions can be readily purified to yield compositions having specific activities of approximately 25,000 or 30,000 Unit/mg protein hyaluronidase activity, and being substantially free of non-PEGylated rHuPH20 (less than 5% non-PEGylated).

Using various PEG reagents, exemplary versions of hyaluronan-degrading enzymes, in particular soluble human recombinant hyaluronidases (e.g. rHuPH20), can be prepared, for example, using mPEG-SBA (30 kD), mPEG-SMB (30 kD), and branched versions based on mPEG2-NHS (40 kD) and mPEG2-NHS (60 kD). PEGylated versions of rHuPH20 have been generated using NHS chemistries, as well as carbonates, and aldehydes, using each of the following reagents: mPEG2-NHS-40K branched, mPEG-NHS-10K branched, mPEG-NHS-20K branched, mPEG2-NHS-60K branched; mPEG-SBA-5K, mPEG-SBA-20K, mPEG-SBA-30K; mPEG-SMB-20K, mPEG-SMB-30K; mPEG-butyrldehyde; mPEG-SPA-20K, mPEG-SPA-30K; and PEG-NHS-5K-biotin. PEGylated hyaluronidases have also been prepared using PEG reagents available from Dowpharma, a division of Dow Chemical Corporation: including hyaluronidases PEGylated with Dowpharma's p-nitrophenyl-carbonate PEG (30 kDa) and with propionaldehyde PEG (30 kDa).

In one example, the PEGylation includes conjugation of mPEG-SBA, for example, mPEG-SBA-30K (having a molecular weight of about 30 kDa) or another succinimidyl esters of PEG butanoic acid derivative, to a soluble hyaluronidase. Succinimidyl esters of PEG butanoic acid derivatives, such as mPEG-SBA-30K readily couple to amino groups of proteins. For example, covalent conjugation of m-PEG-SBA-30K and rHuPH20 (which is approximately 60 KDa in size) provides stable amide bonds between rHuPH20 and mPEG, as shown in Scheme 1, below.

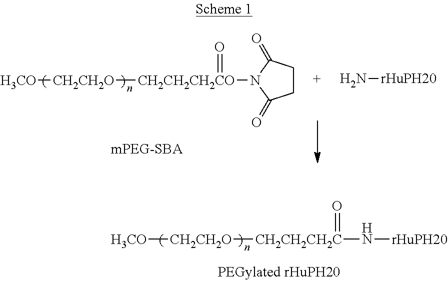

Typically, the mPEG-SBA-30K or other PEG is added to the hyaluronan-degrading enzyme, in some instances a hyaluronidase, at a PEG:polypeptide molar ratio of 10:1 in a suitable buffer, e.g. 130 mM NaCl/10 mM HEPES at pH 6.8 or 70 mM phosphate buffer, pH 7, followed by sterilization, e.g. sterile filtration, and continued conjugation, for example, with stirring, overnight at 4° C. in a cold room. In one example, the conjugated PEG-hyaluronan-degrading enzyme is concentrated and buffer-exchanged.

Other methods of coupling succinimidyl esters of PEG butanoic acid derivatives, such as mPEG-SBA-30K are known in the art (see, e.g. U.S. Pat. No. 5,672,662; U.S. Pat. No. 6,737,505; and U.S. Pub. No. 2004/0235734). For example, a polypeptide, such as a hyaluronan-degrading enzyme (e.g. a hyaluronidase), can be coupled to an NHS activated PEG derivative by reaction in a borate buffer (0.1 M, pH 8.0) for one hour at 4° C. The resulting PEGylated protein can be purified by ultrafiltration. Alternatively, PEGylation of a bovine alkaline phosphatase can be accomplished by mixing the phosphatase with mPEG-SBA in a buffer containing 0.2 M sodium phosphate and 0.5 M NaCl (pH 7.5) at 4° C. for 30 minutes. Unreacted PEG can be removed by ultrafiltration. Another method reacts polypeptide with mPEG-SBA in deionized water to which triethylamine is added to raise the pH to 7.2-9. The resulting mixture is stirred at room temperature for several hours to complete the PEGylation.

Methods for PEGylation of hyaluronan-degrading polypeptides, including, for example, animal-derived hyaluronidases and bacterial hyaluronan-degrading enzymes, are known to one of skill in the art. See, for example, European Patent No. EP 0400472, which describes the PEGylation of bovine testes hyaluorindase and chondroitin ABC lyase. Also, U.S. Publication No. 2006/014968 describes PEGylation of a human hyaluronidase derived from human PH20. For example, the PEGylated hyakironan-degrading enzyme generally contains at least 3 PEG moieties per molecule. For example, the hyaluronan-degrading enzyme can have a PEG to protein molar ratio between 5:1 and 9:1, for example, 7:1.

### E. PHARMACEUTICAL COMPOSITIONS AND FORMULATIONS

Provided herein are compositions of a polymer-conjugated hyaluronan-degrading enzyme. Such compositions can be used in the methods herein to reduce or ameliorate insulin resistance or to prevent or ameliorate a disease or condition associated with insulin resistance. The composition can be formulated for parenteral delivery (i.e. for systemic delivery). For example, the compositions provided herein are formulated for subcutaneous delivery or for intravenous delivery. The compositions can be formulated for single dosage administration or for multiple dosage administration. The composition can be formulated for direct administration. The compositions can be provided as a liquid or lyophilized formulation. Where the compositions are provided in lyophilized form they can be reconstituted just prior to use by an appropriate buffer, for example, a sterile saline solution.

The compounds can be formulated into suitable pharmaceutical preparations such as solutions, suspensions, tablets, dispersible tablets, pills, capsules, powders, sustained release formulations or elixirs, for oral administrate, as well as transdermal patch preparation and dry powder inhalers. Typically, the compounds are formulated into pharmaceutical compositions using techniques and procedures well known in the art (see e.g. Ansel Introduction to Pharmaceutical Dosage Forms, Fourth Edition, 1985, 126). Generally, the mode of formulation is a function of the route of administration.

Compositions can be formulated for administration by any route known to those of skill in the art including intramuscular, intravenous, intradermal, intralesional, intraperitoneal injection, subcutaneous, intratumoral, epidural, nasal, oral, vaginal, rectal, topical, local, otic, inhalational, buccal (e.g. sublingual), and transdermal administration or any route. Other modes of administration also are contemplated. Administration can be local, topical or systemic depending upon the locus of treatment. Local administration to an area in need of treatment can be achieved by, for example, but not limited to, local infusion during surgery, topical application, e.g. in conjunction with a wound dressing after surgery, by injection, by means of a catheter, by means of a suppository, or by means of an implant.

Compositions also can be administered with other biologically active agents, either sequentially, intermittently or in the same composition. Administration also can include controlled release systems including controlled release formulations and device controlled release, such as by means of a pump.

The most suitable route in any given case depends on a variety of factors, such as the nature of the disease, the progress of the disease, the severity of the disease the particular composition which is used. For purposes herein, it is desired that a polymer-conjugated hyaluronan-degrading enzyme, such as a soluble hyaluronidase is administered such that a pharmaceutically available amount or level exists in the plasma. For example, compostions are administered sytemically, for example, via intravenous administration. Subutaneous methods also can be employed, although increased absorption times can be necessary to ensure equivalent bioavailability compared to intravenous methods. Pharmaceutical compositions can be formulated in dosage forms appropriate for each route of administration.

Administration methods can be employed to decrease the exposure of hyaluronan-degrading enzymes, e.g. soluble hyaluronidases, and other molecules to degradative processes, such as proteolytic degradation and immunological intervention via antigenic and immunogenic responses. Examples of such methods include local administration at the site of treatment.

1. Formulations

Pharmaceutically acceptable compositions are prepared in view of approvals for a regulatory agency or other agency prepared in accordance with generally recognized pharmacopeia for use in animals and in humans. Compositions can take the form of solutions, suspensions, emulsion, tablets, pills, capsules, powders, and sustained release formulations. A composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, and other such agents. The formulation should suit the mode of administration.

Pharmaceutical compositions can include carriers such as a diluent, adjuvant, excipient, or vehicle with which an enzyme or activator is administered. Examples of suitable pharmaceutical carriers are described in “Remington's Pharmaceutical Sciences” by E. W. Martin. Such compositions will contain a therapeutically effective amount of the compound, generally in purified form, together with a suitable amount of carrier so as to provide the form for proper administration to the patient. Such pharmaceutical carriers can be sterile liquids, such as water and oils, including those of petroleum, animal, vegetable or synthetic origin, such as peanut oil, soybean oil, mineral oil, and sesame oil. Water is a typical carrier when the pharmaceutical composition is administered intravenously. Saline solutions and aqueous dextrose and glycerol solutions also can be employed as liquid carriers, particularly for injectable solutions. Compositions can contain along with an active ingredient: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polyinylpyrrolidine, celluloses and derivatives thereof, povidone, crospovidones and other such binders known to those of skill in the art. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. A composition, if desired, also can contain minor amounts of wetting or emulsifying agents, or pH buffering agents, for example, acetate, sodium citrate, cyclodextrine derivatives, sorbitan monolaurate, triethanolamine sodium acetate, triethanolamine oleate, and other such agents.

In one example, pharmaceutical preparation can be in liquid form, for example, solutions, syrups or suspensions. Such liquid preparations can be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. almond oil, oily esters, or fractionated vegetable oils); and preservatives (e.g. methyl or propyl-p-hydroxybenzoates or sorbic acid). In another example, pharmaceutical preparations can be presented in lyophilized form for reconstitution with water or other suitable vehicle before use.

Pharmaceutically therapeutically active compounds and derivatives thereof are typically formulated and administered in unit dosage forms or multiple dosage forms. Each unit dose contains a predetermined quantity of therapeutically active compound sufficient to produce the desired therapeutic effect, in association with the required pharmaceutical carrier, vehicle or diluent. Unit dosage forms, include, but are not limited to, tablets, capsules, pills, powders, granules, sterile parenteral solutions or suspensions, and oral solutions or suspensions, and oil water emulsions containing suitable quantities of the compounds or pharmaceutically acceptable derivatives thereof. Unit dose forms can be contained ampoules and syringes or individually packaged tablets or capsules. Unit dose forms can be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses that are not segregated in packaging. Generally, dosage forms or compositions containing active ingredient in the range of 0.005% to 100% with the balance made up from non-toxic carrier can be prepared.

Pharmaceutical composition can be formulated in dosage forms appropriate for each route of administration.

a. Injectables, Solutions and Emulsions

Parenteral administration, generally characterized by injection, either subcutaneously, intramuscularly, intratumorally, intravenously or intradermally is contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain an activator in the form of a solvent such as pH buffering agents, metal ion salts, or other such buffers. The pharmaceutical compositions also may contain other minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see, e.g. U.S. Pat. No. 3,710,795) also is contemplated herein. The percentage of active compound contained in such parenteral compositions is highly dependent on the specific nature thereof, as well as the activity of the compound and the needs of the subject.

For example, a standard stabilized formulation of a polymer-conjugated hyaluronan-degrading enzyme, such as a polymer-conjugated soluble hyaluronidase as provided herein, is formulated with one or more of EDTA, NaCl, CaCl₂ histidine, lactose, albumin, Pluronic® F68, TWEEN® and/or other detergent or other similar agents. For example, compositions provided herein can contain one or more pH buffers (such as, for example, histidine, phosphate, or other buffers), or acidic buffer (such as acetate, citrate, pyruvate, Gly-HCl, succinate, lactate, maleate or other buffers), tonicity modifier (such as, for example, an amino acid, polyalcohol, NaCl, trehalose, other salts and/or sugars), stabilizer, chelating agent, such as ethylenediaminetetraacetic acid, ethylenediaminetetraacetate or calcium EDTA, oxygen scavenger, such as methionine, ascorbic acid/ascorbate, citric acid/citrate, or albumin, and/or a preservative, such as preservative containing an aromatic ring (e.g. phenol or cresol). Exemplary stabilizers that are useful for compositions containing a hyaluronan-degrading enzyme include detergents, such as polysorbates and proteins such as human serum albumin (SEQ ID NO:211). Exemplary concentrations of serum albumin that are useful in the compositions herein include 0.1 mg/mL to 1 mg/mL, such as at least or at least about or about or 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL or 1 mg/mL, but can be more or less. Polysorbates also can be present in the compositions at, for example, concentrations of or about between 0.001% to 0.1%, such as at least about or at least or about or 0.001%, 0.002%, 0.003%, 0.004%, 0.005%, 0.006%, 00.007%, 0.008%, 0.009%, 0.01%, 0.02%, 0.03%, 0.04%, 0.05%, 0.06%, 0.07%, 0.08%, 0.09% or 0.1%. A metal chelating agent, such as calcium EDTA (CaEDTA), also can be present, such as for example, at concentrations of between approximately 0.02 mM to 20 mM, such as at least about or at least or about or 0.02 mM, 0.04 mM, 0.06 mM, 0.08 mM, 0.1 mM, 0.2 mM, 0.3 mM, 0.4 mM, 0.5 mM, 0.6 mM, 0.7 mM, 0.8 mM, 0.9 mM, 1 mM, 5 mM, 10 mM, 15 mM, 20 mM or more. The pH and the osmolarity of the compositions can be adjusted by one of skill in the art to optimize the conditions for the desired activity and stability of the composition. In some examples, the compositions provided herein have an osmolarity of between 100 mOsm/kg to 500 mOsm/kg, such as at least or at least about or at or about 100 mOsm/kg, 120 mOsm/kg, 140 mOsm/kg, 160 mOsm/kg, 180 mOsm/kg, 200 mOsm/kg, 220 mOsm/kg, 240 mOsm/kg, 260 mOsm/kg, 280 mOsm/kg, 300 mOsm/kg, 320 mOsm/kg, 340 mOsm/kg, 360 mOsm/kg, 380 mOsm/kg, 400 mOsm/kg, 420 mOsm/kg, 440 mOsm/kg, 460 mOsm/kg, 500 or more mOsm/kg, and a pH of between or between about 6 to 8, such as 6 to 7.4, for example at or about 6, 6.2, 6.4, 6.6, 6.8, 7, 7.2, 7.4, 7.6, 7.8 or 8.

Generally, NaCl is provided in formulations containing a hyaluronan-degrading enzyme herein, for example, in an amount that is or is about 100 mM-150 mM or more. For example, an exemplary formulation can contain at or about 10 mM histidine and/or at or about 130 mM NaCl. Other formulations can contain in addition or alternatively lactose, for example, at or about 13 mg/ml. Additionally, an anti-bacterial or anti-fungal agent, including, but not limited to thiomersal, can be present in the formulation. Formulations can further contain Albumin, Pluronic® F68, TWEEN® and/or other detergent. The formulations are provided at a pH that is or is about 6.0 to 7.4, such as 6.0, 6.1., 6.2, 6.3, 6.4, 6.5, 6.6, 6.7, 6.8, 6.9, 7.0, 7.1, 7.2, 7.3 or 7.4, generally that is or is about pH 6.5. Concentrated formulations of hyaluronan-degrading enzyme for use herein are generally diluted in a saline solution or other salt buffered solution prior administration to maintain the appropriate salt concentration.

Injectables are designed for local and systemic administration. For purposes herein, local administration is desired for direct administration to the affected skeletal muscle associated with accumulated or excess hyaluronan. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous. If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers, which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEENs 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment. If administered intravenously, suitable carriers include physiological saline or phosphate buffered saline (PBS), and solutions containing thickening and solubilizing agents, such as glucose, polyethylene glycol, and polypropylene glycol and mixtures thereof.

The concentration of the pharmaceutically active compound is adjusted so that an injection provides an effective amount to produce the desired pharmacological effect. The exact dose depends on the age, weight and condition of the patient or animal as is known in the art. The unit-dose parenteral preparations are packaged in an ampoule, a vial or a syringe with a needle. The volume of liquid solution or reconstituted powder preparation, containing the pharmaceutically active compound, is a function of the disease to be treated and the particular article of manufacture chosen for package. All preparations for parenteral administration must be sterile, as is known and practiced in the art.

b. Lyophilized Powders

Of interest herein are lyophilized powders, which can be reconstituted for administration as solutions, emulsions and other mixtures. They may also be reconstituted and formulated as solids or gels. The lyophilized powders can be prepared from any of the solutions described above.

The sterile, lyophilized powder is prepared by dissolving a compound of a soluble hyaluronidase and/or second agent in a buffer solution. The buffer solution may contain an excipient which improves the stability or other pharmacological component of the powder or reconstituted solution, prepared from the powder. Subsequent sterile filtration of the solution followed by lyophilization under standard conditions known to those of skill in the art provides the desired formulation. Briefly, the lyophilized powder is prepared by dissolving an excipient, such as dextrose, sorbital, fructose, corn syrup, xylitol, glycerin, glucose, sucrose or other suitable agent, in a suitable buffer, such as citrate, sodium or potassium phosphate or other such buffer known to those of skill in the art. Then, a selected enzyme is added to the resulting mixture, and stirred until it dissolves. The resulting mixture is sterile filtered or treated to remove particulates and to insure sterility, and apportioned into vials for lyophilization. Each vial will contain a single dosage (e.g. 1 mg-1 g, generally 1-100 mg, such as 1-5 mg) or multiple dosages of the compound. The lyophilized powder can be stored under appropriate conditions, such as at about 4° C. to room temperature.

Reconstitution of this lyophilized powder with a buffer solution provides a formulation for use in parenteral administration. The precise amount depends upon the indication treated and selected compound. Such amount can be empirically determined.

c. Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation (see, e.g. U.S. Pat. Nos. 4,044,126; 4,414,209; and 4,364,923, which describe aerosols for delivery of a steroid useful for treatment inflammatory diseases, particularly asthma). These formulations for administration to the respiratory tract can be in the form of an aerosol or solution for a nebulizer, or as a microtine powder for insufflation, alone or in combination with an inert carrier such as lactose. In such a case, the particles of the formulation will typically diameters of less than 50 microns, preferably less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients also can be administered.

Formulations suitable for transdermal administration are provided. They can be provided in any suitable format, such as discrete patches adapted to remain in intimate contact with the epidermis of the recipient for a prolonged period of time. Such patches contain the active compound in optionally buffered aqueous solution of, for example, 0.1 to 0.2M concentration with respect to the active compound. Formulations suitable for transdermal administration also can be delivered by iontophoresis (see, e.g. Pharmaceutical Research 3(6):318 (1986)) and typically take the form of an optionally buffered aqueous solution of the active compound.

d. Compositions for Other Routes of Administration

Depending upon the condition treated other routes of administration, such as topical application, transdermal patches, oral and rectal administration also are contemplated herein. For example, pharmaceutical dosage forms for rectal administration are rectal suppositories, capsules and tablets for systemic effect. Rectal suppositories include solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 mg. Tablets and capsules for rectal administration are manufactured using the same pharmaceutically acceptable substance and by the same methods as for formulations for oral administration.

Formulations suitable for rectal administration can be provided as unit dose suppositories. These can be prepared by admixing the active compound with one or more conventional solid carriers, for example, cocoa butter, and then shaping the resulting mixture.

For oral administration, pharmaceutical compositions can take the form of, for example, tablets or capsules prepared by conventional means with pharmaceutically acceptable excipients such as binding agents (e.g. pregelatinized maize starch, polyvinyl pyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycolate); or wetting agents (e.g. sodium lauryl sulphate). The tablets can be coated by methods well-known in the art.

Formulations suitable for buccal (sublingual) administration include, for example, lozenges containing the active compound in a flavored base, usually sucrose and acacia or tragacanth; and pastilles containing the compound in an inert base such as gelatin and glycerin or sucrose and acacia.

Pharmaceutical compositions also can be administered by controlled release formulations and/or delivery devices (see, e.g. in U.S. Pat. Nos. 3,536,809; 3,598,123; 3,630,200; 3,845,770; 3,847,770; 3,916,899; 4,008,719; 4,687,610; 4,769,027; 5,059,595; 5,073,543; 5,120,548; 5,354,566; 5,591,767; 5,639,476; 5,674,533 and 5,733,566).

Various delivery systems are known and can be used to administer selected compositions, such as but not limited to, encapsulation in liposomes, microparticles, microcapsules, recombinant cells capable of expressing the compound, receptor mediated endocytosis, and delivery of nucleic acid molecules encoding a soluble hyaluronidase or other agent such as retrovirus delivery systems.

Hence, in certain embodiments, liposomes and/or nanoparticles also can be employed with administration of compositions herein. Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 angstroms containing an aqueous solution in the core.

Phospholipids can form a variety of structures other than liposomes when dispersed in water, depending on the molar ratio of lipid to water. At low ratios, the liposomes form. Physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

Liposomes interact with cells via different mechanisms: endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. Varying the liposome formulation can alter which mechanism is operative, although more than one can operate at the same time. Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use herein, and such particles can be easily made.

2. Formulation Amounts

The compositions can be formulated for single dosage administration or for multiple dosage administration. The agents can be formulated for direct administration.

In the compositions provided herein, the polymer-conjugated hyaluronan-degrading enzyme is formulated in an amount for direct administration in a range between or about between 0.5 μg to 50 mg, such as 100 μg to 1 mg, 1 mg to 20 mg, 100 μg to 5 mg, 0.5 μg to 1450 μg, 1 μg to 1000 μg, 5 μg to 1250 μg, 10 μg to 750 μg, 50 μg to 500 μg, 0.5 μg to 500 μg, 500 μg to 1450 μg. For example, the polymer-conjugated hyaluronan-degrading enzyme is formulated in an amount for direct administration in a range between or about between 15 Units (U) or 150 Units (U) to 60,000 Units per dose, 300 U to 30,0000 U, 500 U to 25,000 U, 500 U to 10,000 U, 150 U to 15,000 U, 150 U to 5000 U, 500 U to 1000 U, 5000 U to 45,000 U 10,000 U to 50,000 U or 20,000 U to 60,000 U, for example at least or about at least or about or 15 U, 50 U, 100 U, 200 U, 300 U; 400 U; 500 U; 600 U; 700 U; 800 U; 900 U; 1,000 U; 1250 U; 1500 U; 2000 U; 3000 U; 4000 U; 5,000 U; 6,000 U; 7,000 U; 8,000 U; 9,000 U; 10,000 U; 20,000 U; 30,000 U; 40,000 U; or 50,000 U. The polymer-conjugated hyaluronan-degrading enzyme can be provided as a stock solution at or about 50 U/mL to 15,000 U/mL, such as 10 U/mL to 500 U/mL, 1000 U/mL to 15,000 U/mL, 100 U/mL to 5,000 U/mL, 500 U/mL to 5,000 U/mL or 100 U/mL to 400 U/mL, for example at least or at least about or about or 50 U/mL, 100 U/mL, 150 U/mL, 200 U/mL, 400 U/mL, 500 U/mL, 1000 U/mL, 2000 Units/mL, 3000 U/mL, 4000 U/mL, 5000 U/mL, 6000 U/mL, 7000 U/mL, 8000 U/mL, 9000 U/mL, 10,000 U/mL, 11,000 U/mL, 12,000 U/mL, or 12,800 U/mL. The volume of the composition can be 0.5 mL to 1000 mL, such as 0.5 mL to 100 mL, 0.5 mL to 10 mL, 1 mL to 500 mL, 1 mL to 10 mL, such as at least or about at least or about or 0.5 mL, 1 mL, 2 mL, 3 mL, 4 mL, 5 mL, 6 mL, 7 mL, 8 mL, 9 mL, 10 mL, 15 mL, 20 mL, 30 mL, 40 mL, 50 mL or more. The composition is generally formulated so that the polymer-conjugated hyaluronan-degrading enzyme is not administered in volumes greater than about 50 mL, and typically is administered in a volume of 5-30 mL, generally in a volume that is not greater then about 10 mL. For larger volumes, the time of infusion can be adapted to facilitate delivery of the larger volume. For example, infusion time can be at least 1 minute, 5 minutes, 10 minutes, 20 minutes, 30 minutes, 40 minutes, 50 minutes, 1 hour or more.

3. Packaging and Articles of Manufacture

Also provided are articles of manufacture containing packaging materials, any pharmaceutical composition or combination provided herein, and a label that indicates that the compositions and combinations are to be used for treatment of cancers, such as stromal tumor cancers or solid tumor cancers. Exemplary of articles of manufacture are containers including single chamber and dual chamber containers. The containers include, but are not limited to, tubes, bottles and syringes. The containers can further include a needle for subcutaneous administration.

In one example, the article of manufacture contains a pharmaceutical composition containing the polymer-conjugated hyaluronan-degrading enzyme. In another example, the article of manufacture contains pharmaceutical compositions containing the polymer-conjugated hyaluronan-degrading enzyme and a further agent (e.g. therapeutic agent). In this example, the agents can be provided together or separately, for packaging as articles of manufacture.

The articles of manufacture provided herein contain packaging materials. Packaging materials for use in packaging pharmaceutical products are well known to those of skill in the art. See, for example, U.S. Pat. Nos. 5,323,907; 5,052,558; and 5,033,252, each of which is incorporated herein in its entirety. Examples of pharmaceutical packaging materials include, but are not limited to, blister packs, bottles; tubes, inhalers, pumps, bags, vials, containers, syringes, bottles, and any packaging material suitable for a selected formulation and intended mode of administration and treatment.

The choice of package depends on the agents, and whether such compositions will be packaged together or separately. In general, the packaging is non-reactive with the compositions contained therein. In other examples, some of the components can be packaged as a mixture. In other examples, all components are packaged separately. Thus, for example, the components can be packaged as separate compositions that, upon mixing just prior to administration, can be directly administered together. Alternatively, the components can be packaged as separate compositions for administration separately.

The components can be packaged in a container. The components are separately packaged in the same container. Generally, examples of such containers include those that have an enclosed, defined space that contains the polymer-conjugated hyaluronan-degrading enzyme, and a separate enclosed, defined space containing the other components or component such that the subsequent areas are separated by a readily removable membrane which, upon removal, permits the components to mix, or which permits the components to be separately administered.

Any container or other article of manufacture is contemplated, so long as the agents are separated from the other components prior to administration. For suitable embodiments see, e.g. containers described in U.S. Pat. Nos. 3,539,794 and 5,171,081.

Selected compositions including articles of manufacture thereof also can be provided as kits. Kits can include a pharmaceutical composition described herein and an item for administration provided as an article of manufacture. The kit can, optionally, include instructions for application including dosages, dosing regimens and instructions for modes of administration. Kits also can include a pharmaceutical composition described herein and an item for diagnosis.

### F. METHODS OF ASSESSING ACTIVITY, BIOAVAILABILITY AND PHARMACOKINETICS

Hyaluronan-degrading enzymes, such as polymer-conjugated hyaluronan-degrading enzyme, for example a hyaluronidase (e.g. PH20 or PEGPH20) can be used to reduce HA levels and can act as a therapeutic agent for the treatment of insulin-resistant-associated diseases and conditions. Assays can be used to assess the in vitro and in vivo activity of a hyaluronan-degrading enzyme. Included among such assays are those that assess the pharmacokinetic and pharmacodynamic properties of a hyaluronan-degrading enzyme, including bioavailability, and tolerability. The activity of a hyaluronan-degrading enzyme can be assessed to determine the effect on insulin action. Assays can also be used to determine insulin action as a result of treatment with a hyaluronan-degrading enzyme. Assays to assess the biological activity of both insulin and a hyaluronan-degrading enzyme are well known in the art. Such assays can be used, for example, to determine appropriate dosages of a hyaluronan-degrading enzyme and the frequency of dosing, for treatment of insulin resistance. This section provides a description of methods that can be used to assess efficacy, responsiveness, tolerability and/or pharmacokinetics of a polymer-conjugated hyaluronan-degrading enzyme therapy.

1. In Vitro Assays

a. Hyaluronidase Activity

The activity of a hyaluronan-degrading enzyme can be assessed using methods well known in the art. Such assays can include, but are not limited to, measuring amounts of HA in tissue, such as by immunohistochemistry; measurements of soluble HA in plasma; measurements of HA catabolites in blood or urine; or measurements of hyaluronidase activity in plasma. The Examples below exemplify these assays. Other assays such as measurements of pharmacokinetics, methods for which are well known to those of skill in the art, can be used to assess the pharmacokinetic parameters of HA administration.

For example, the USP XXII assay for hyaluronidase determines hyaluronidase activity indirectly by measuring the amount of undegraded hyaluronic acid, or hyaluronan (HA), substrate remaining after the enzyme is allowed to react with the HA for 30 min at 37° C. (USP XXII-NF XVII (1990) 644-645, United States Pharmacopeia Convention, Inc., Rockville, Md.). A Hyaluronidase Reference Standard (USP) or National Formulary (NF) Standard Hyaluronidase solution can be used in an assay to ascertain the activity, in units, of any hyaluronidase. In one example, activity is measured using a microturbididy assay. This is based on the formation of an insoluble precipitate when HA binds with serum albumin. The activity is measured by incubating hyaluronidase with sodium hyaluronate (hyaluronic acid) for a set period of time (e.g. 10 minutes) and then precipitating the undigested sodium hyaluronate with the addition of acidified serum albumin. The turbidity of the resulting sample is measured at 640 nm after an additional development period. The decrease in turbidity resulting from hyaluronidase activity on the sodium hyaluronate substrate is a measure of hyaluronidase enzymatic activity.

In another example, hyaluronidase activity is measured using a microtiter assay in which residual biotinylated HA is measured following incubation with hyaluronidase (see, e.g. Frost and Stem (1997) Anal. Biochem. 251:263-269 and U.S. Pub. No. 2005/0260186). The free carboxyl groups on the glucuronic acid residues of HA are biotinylated, and the biotinylated HA substrate is covalently coupled to a microtiter plate. Following incubation with hyaluronidase, the residual biotinylated HA substrate is detected using an avidin-peroxidase reaction and compared to that obtained following reaction with hyaluronidase standards of known activity.

In another example, blood and urine can be collected at different time points throughout HA administration and assayed for catabolites of hyaluronan. The presence of catabolites is indicative of the degradation of hyaluronan and is thus a measure of the activity of hyaluronidase. Plasma enzyme also can be assessed and measured over time following administration. For example, HA catabolites, which are HA-disaccharide breakdown products, can be assessed using high-performance liquid chromatography (HPLC) to separate and measure saccharide peak areas.

Other assays various assays to assess hyaluronidase activity, including effects on HA synthesis or degradation, are known to one of skill in the art, including but not limited to any described herein or known in the art, for example, in vitro assays that measure HA degradation (see, e.g. Frost and Stem (1997) Anal. Biochem. 251:263-269); staining tissue or other samples for HA, such as by using an HA-binding protein or other anti-HA reagent (see, e.g. Nishida et al. (1999) J. Biol. Chem. 274:21893-21899); particle exclusion assay (Nishida et al. (1999); Morohashi et al. (2006) Biochem Biophys. Res. Comm. 345:1454-1459); or measuring or assessing HAS mRNA expression for an has gene (Nishida et al. (1999)). Other assays to measure hyaluronidase activity are known in the art and can be used in the methods provided herein (see, e.g. Delpech et al. (1995) Anal. Biochem. 229:35-41; Takahashi et al. (2003) Anal. Biochem. 322:257-263).

The ability of a modified hyaluronan-degrading enzyme, such as a polymer-conjugated hyaluronan-degrading enzyme, for example a hyaluronidase, including pegylated hyaluronidase, to exhibit therapeutic activity for reducing or prevening insulin resistance can be assessed using any one or more of the assays described above. For example, the ability of a polymer-conjugated hyaluronan-degrading enzyme, such as a hyaluronidase, for example PH20 (e.g. PEGPH20) to reduce hyaluronan levels or content can be assessed using any one or more of the assays above in vitro, ex vivo and/or in vivo. In one example, a polymer-conjugated hyaluronan-degrading enzyme can be administered to a subject with insulin resistance, or an appropriate animal model, and the effect on hyaluronan levels and insulin action can be assessed and compared to subjects or animal models not administered a hyaluronan-degrading enzyme.

b. Insulin Action

The ability of insulin to stimulate glucose uptake, such as in muscle, and suppress glucose production, for example hepatic glucose production, can be assessed in vitro. For example, in vitro assays well known in the art can be performed to assess the ability of insulin to stimulate glucose uptake or affect any other of its typical metabolic outcomes. In one method used to measure insulin-stimulated glucose uptake, adipocytes are incubated with labeled glucose, such as 2-deoxy-D-[2,6³-H]glucose or D[U-¹⁴C]glucose with or without insulin. The incorporated radioactivity is then measured to determine the amount of glucose uptake in the presence or absence of insulin (Louveau et al. (2004) J. Endocrinol. 181:271-280; Duttaroy et al. (2005) Diabetes 54:251-258). In vitro assays to assess glucose production in H4IIE cells in the presence of insulin also can be performed (Wang et al. (2000) J. Biochem. 275:14717-14721; Duttaroy et al. (2005) Diabetes 54:251-258).

In another method, in vitro quantitative measurement of insulin in human serum and plasma can be measured using a solid phase enzyme-linked immunosorbant assay (ELISA). In the assay, wells coated with a monoclonal antibody with high activity for insulin are incubated with the sample, control, and an enzyme conjugate, an antibody linked to horseradish peroxidase, which will form a sandwich complex bound to the well. Unbound conjugates are then washed off with wash buffer. The amount of bound peroxidase is proportional to the concentration of the insulin present in the sample. Upon addition of the substrate and chromogen, the intensity of color will develop in proportion to the concentration of insulin in the sample.

2. In Vivo Animal Models

Animal models can be used to assess the effects of a hyaluronan-degrading enzyme or polymer-conjugated hyaluronan-degrading enzyme as provided herein on HA levels or content. The ability of insulin to stimulate glucose uptake, such as in muscle, and suppress glucose production, for example hepatic glucose production, also can be assessed in vivo. Animal models can be used to assess the effects of administration of a hyaluronan-degrading enzyme on insulin action, for example in an animal that is insulin resistant. In addition, animal models can be used to assess the pharmacokinetics or tolerability of the hyaluronan-degrading enzyme or modified hyaluronan-degrading enzyme.

Animal models can include, but are not limited to, mice, rats, rabbits, dogs, guinea pigs and non-human primate models, such as cynomolgus monkeys or rhesus macaques. Animal models include genetic models as well as diet-induced insulin-resistant models. For example, diet-induced insulin-resistant models include those in which, prior to administering a hyaluronidase-degrading enzyme, diet-induced insulin resistance can be established in suitable test animals, e.g. mice. In some examples, mice, such as C57/BL67 mice, are fed a high fat diet, such as a diet that contains 60% calories as fat, over an extended period of time, for example two weeks, to establish an animal model of insulin resistance. Exemplary of an animal model of diet-induced insulin resistance involves the development of insulin resistance in animals such as C57/BL67 mice after two weeks on a high fat (60% calories as fat) diet.

Genetic models also can be used in which animals are rendered to be deficient in one or more genes that results in insulin resistance. Such genetically engineered mouse models (GEMM) can recapitulate the molecular and clinical features of a disease or disorder. For example, exemplary insulin resistant genetic mouse models can result from knockout or mutagenesis of the insulin receptor (Insr), knockout of type 1 insulin-like growth factor receptor (Igf1r), or mutations of genes encoding proteins in the insulin signaling pathway, such as Irs proteins (Nandi et al. (2003) Physiol. Rev. 84:623-647). These mice develop insulin resistance and can exhibit features similar to the human disorder.

The hyaluronan-degrading enzymes provided herein can be administered to the mice to assess effects on insulin resistance. For example, HA levels can be assessed or measured. In another example, the effects of administering a hyaluronan-degrading enzyme on glucose uptake or hepatic liver production can be assessed.

3. Ex Vivo Assays

Insulin activity and the effects of administration of a hyaluronan-degrading enzyme, such as a pegylated hyaluronidase, can be measured using various ex vivo techniques that are well known in the art.

In one example, insulin action can be assessed by measuring Akt signaling in muscle, such as in skeletal muscle, as described in Example 11. Protein expression of Akt and phosphorylated Akt can be assessed by Western blotting in muscle, for example skeletal muscle, after performing a biopsy to obtain a muscle sample, such as gastrocnemius or superficial vastus lateralus (SVL) muscle. The biopsy can be performed after treatment with a hyaluronan-degrading enzyme, and phosphorylation of Akt and total Akt can be assessed in the biopsied muscle. Increased phosphorylation and total Akt as compared to a control correlates to increased levels of insulin action.

In another example, collagen levels, such as collagen IV (ColIV) levels, and vascularization can be assessed in muscle, such as skeletal muscle, to assess the effect of administration of a hyaluronan-degrading enzyme on insulin action. Immunohistochemistry can be used to assess the level of collagen, for example ColIV, in biopsied skeletal muscle, such as gastrocnemius, as exemplified in Example 12. Vascularization can be assessed by determining the percent cardiac output to muscle, i.e. the skeletal muscle blood flow. Increased muscle vascularization can be shown by an increase in expression of the vascular marker CD31 and increased cardiac output to muscle, such as skeletal muscle, as compared to a control.

Glucose uptake in isolated muscle, such as isolated soleus or extensor digitorum longus (EDL) muscle, can be determined to assess the effects of administration of a hyaluronan-degrading enzyme on insulin action. [³H]2DG uptake can be measured in isolated muscle to determine insulin action (Jorgensen et al. (2004) J. Biol. Chem. 279:1070-1079). Example 13 exemplifies this assay.

In another example, adipose cells can be assessed to determine insulin action after administration of a hyaluronan-degrading enzyme, such as a pegylated hyaluronidase. These methods are exemplified in Example 14. Adipose cell size and distribution in adipose tissue can be measured and visualized after staining the tissue (Salans et al. (1968) J. Clin. Invest. 47:153-165). RNA can be isolated and assessed for expression of inflammation- and macrophage-specific genes (Livak et al. (2001) Methods. 25:402-408).

The ability of a polymer-conjugated hyaluronan-degrading enzyme, such as a hyaluronidase, including pegylated hyaluronidase, to affect any one or more of the markers associated with insulin resistance described above, or any other associated markers or phenotypes, can be assessed using any one or more of the assays described above. The methods can be used to assess insulin action in any subject with insulin resistance, including insulin resistance induced by diet, such as a high fat diet.

4. Pharmacokinetics, Pharmacodynamics and Tolerability

Pharmacokinetic or pharmacodynamic studies can be performed using animal models or can be performed during studies with patients to assess the pharmacokinetic properties of a hyaluronan-degrading enzyme, such as a hyaluronidase or modified hyaluronidase (e.g. PEGPH20). Animal models include, but are not limited to, mice, rats, rabbits, dogs, guinea pigs and non-human primate models, such as cynomolgus monkeys or rhesus macaques. In some instances, pharmacokinetic or pharmacodynamic studies are performed using healthy animals. In other examples, the studies are performed using animal models of a disease or disorder for which therapy with a hyaluronan-degrading enzyme is considered, such as animal models of any hyaluronan-associated disease or disorder, for example animal models of insulin resistance.

The pharmacokinetic properties of a hyaluronan-degrading enzyme, such as a modified hyaluronidase, can be assessed by measuring such parameters as the maximum (peak) concentration (Cₘₐₓ), the peak time (i.e. when maximum concentration occurs; Tₘₐₓ), the minimum concentration (i.e. the minimum concentration between doses; Cₘᵢₙ), the elimination half-life (T_{1/2}), and area under the curve (i.e. the area under the curve generated by plotting time versus concentration; AUC), following administration. The absolute bioavailability of the hyaluronidase can be determined by comparing the area under the curve of hyaluronidase following subcutaneous delivery (AUC_{sc}) with the AUC of hyaluronidase following intravenous delivery (AUCᵢᵥ). Absolute bioavailability (F), can be calculated using the formula: F=([AUC]_{sc}×dose_{sc})/([AUC]ᵢᵥ×doseᵢᵥ). A range of doses and different dosing frequency of dosing can be administered in the pharmacokinetic studies to assess the effect of increasing or decreasing concentrations hyaluronan-degrading enzyme, such as a hyaluronidase or modified hyaluronidase (e.g. PEGylated PH20) in the dose.

Studies to assess safety and tolerability also are known in the art and can be used herein. Following administration of the hyaluronan-degrading enzyme provided herein, the development of any adverse reactions can be monitored. Adverse reactions can include, but are not limited to, injection site reactions, such as edema or swelling, headache, fever, fatigue, chills, flushing, dizziness, urticaria, wheezing or chest tightness, nausea, vomiting, rigors, back pain, chest pain, muscle cramps, seizures or convulsions, changes in blood pressure and anaphylactic or severe hypersensitivity responses. Typically, a range of doses and different dosing frequencies are be administered in the safety and tolerability studies to assess the effect of increasing or decreasing concentrations of polymer-conjugated hyaluronan-degrading enzyme in the dose.

Exemplary procedures useful for assessing pharmacodynamic effects of administration of a hyaluronan-degrading enzyme on insulin action include glucose clamp techniques (Brehm et al. (2007) in Clin. Diabetes Res.: Methods and Techniques, Ed. Michael Rosen, pp. 43-76, Example 1; Ayala et al. (2006) Diabetes 55:390-397), as described in Examples 9 and 10. In the hyperinsulinemic euglycemic clamp procedure, exogenous insulin is infused to create hyperinsulinemic plasma insulin concentrations, while the plasma glucose concentration is kept constant at the euglycemic level by means of a variable exogenous glucose infusion. The glucose infusion rate (GIR) required to maintain constant glucose levels during the period of hyperinsulinemia provide a measure of the effect of the infused insulin on glucose metabolism. The GIR is a reflection of the amount of glucose being used by the body (i.e. more exogenous glucose needs to be infused to maintain normal blood glucose levels, when the body is using more glucose), and, therefore, the activity of the administered insulin (i.e. increased insulin action results in reduced endogenous glucose output and increased blood glucose utilization, resulting in an overall decline of blood glucose). Thus, such a procedure can be used to assess insulin action as well as to assess the pharmacokinetic and pharmacodynamic properties of a hyaluronan-degrading enzyme administered to treat insulin resistance. For example, properties such as basal fasting glucose, plasma insulin and plasma non-esterified fatty acid concentrations can be measured during the hyperinsulinemic euglycemic clamp to assess insulin activity. The GIR, endogenous glucose production (endoRₐ) and disappearance rate (R_{d}) and muscle glucose uptake index (R_{g}) in muscle, such as in the gastrocnemius and SVL, can be determined during and after the hyperinsulinemic euglycemic clamp to assess the effects of administration of a hyaluronan-degrading enzyme on insulin activity.

### G. THERAPEUTIC USES

Polymer-conjugated hyaluronan-degrading enzyme compositions provided herein, such as a polymer-conjugated hyaluronidase or polymer-conjugated PH20 (e.g. PEGPH20), can be used in methods of treating, ameliorating or reversing insulin resistance. Since many pathological conditions, including those induced by visceral fat obesity, are characterized by insulin resistance, the polymer-conjugated hyaluronan-degrading enzymes provided herein also can be used to prevent the onset or occurrence or ameliorate any metabolic syndrome characterized by insulin resistance, such as diabetes (e.g. type 2 diabetes), cardiovascular disease (e.g. heart disease), dyslipidemias, fatty liver or hypertension.

The therapeutic uses include administration of the polymer-conjugated hyaluronan-degrading enzyme composition to a subject that has insulin resistance. In particular examples, the polymer-conjugated hyaluronan-degrading enzyme is administered in a treatment regime with a corticosteroid in order to ameliorate musculoskeletal side effects that can result upon treatment with a polymer conjugated-hyaluronan-degrading enzyme. The polymer-conjugated hyaluronan-degrading enzyme can be administered alone as the only active ingredient for reducing insulin resistance (with or without a corticosteroid) or in combination with other treatments or agents to ameliorate or reduce insulin resistance. Current pharmaceutical treatments for insulin resistance, including metabolic and cardiovascular disorders, include combinations of lipid-lowering drugs, hypoglycemic drugs, anti-hypertensive agents, diet and exercise.

The methods herein include selecting subjects for treatment (e.g. prior to treatment) that exhibit signs or symptoms of insulin resistance. In some examples of methods herein, subjects also can be selected for that exhibit increased or elevated levels of hyaluronan in skeletal muscle. Such a selection method can select for patients that are likely to be responsive to treatment with a hyaluronan-degrading enzyme. Further, the methods herein include methods to monitor insulin resistance in order to assess and monitor the therapeutic effectiveness of the treatment.

In the methods herein for reducing or ameliorating insulin resistance, or for preventing or ameliorating an associated metabolic syndrome, the polymer-conjugated hyaluronan-degrading enzyme can be administered by any route of administration to effect systemic delivery of the enzyme. The polymer-conjugated hyaluronan-degrading enzyme can be administered subcutaneously, intravenously, intramuscularly or by any other systemic route. Typically, the polymer-hyaluronan-degrading enzyme composition is administered intravenously. In another example, the polymer-conjugated hyaluronan-degrading enzyme is administered locally, for example, intramuscularly.

In the methods provided herein, the polymer-conjugated hyaluronan-degrading enzyme is administered repeatedly to a subject in order to effect chronic reduction in skeletal muscle hyaluronan. The methods of treatment include repeated administration of the polymer-conjugated hyaluronan-degrading enzyme, for example, administration hourly, every several hours, three times daily, twice daily, once daily, every other day, every third day, every week, every other week, every third week, monthly, or other repeated administration. In some examples herein, the methods include continuous administration over a period of time. Typically, as described below, the dosage amount and frequency of administration is such to effect chronic reduction in hyaluronan, and to confer a therapeutic effect by reducing or ameliorating insulin resistance. Methods to assess insulin resistance, including signs and symptoms of insulin resistance, are known to one of skill in the art and can be monitored as described herein. The particular dosage and frequency of administration can be empirically determined.

A non-limiting description of exemplary methods of use a polymer-conjugated hyaluronan-degrading enzymes, such as hyaluronidase enzymes or soluble hyaluronidase enzyme, for example PH20 (e.g. PEGPH20), for treating insulin resistance and other associated diseases and conditions are described below. The description includes non-limiting examples of dosages, dosage regimes, combination treatments, and selection and monitoring techniques and procedures for use in practice of the methods herein. It is understood that the description set forth herein are exemplary only and are not intended to limit the scope thereof.

1. Diseases and Conditions

a. Insulin Resistance

Provided herein are methods of reducing, lessening or ameliorating insulin resistance in a subject by administering a polymer-conjugated hyaluronan-degrading enzyme to a subject. As discussed above, insulin resistance is a disorder of glucose metabolism, whereby cells do not respond to insulin leading to a defect in glucose uptake. Insulin resistant persons have a diminished ability to properly metabolize glucose and respond poorly, if at all, to insulin therapy. Generally, insulin resistance is attributable to obesity (visceral fat obesity) in which fat accumulates around the internal organs. In particular, provided herein are methods of using a polymer-conjugated hyaluronan-degrading (such as a hyaluronidase, for example a PH20 (e.g. PEGPH20)) for treating, ameliorating or reducing obesity-induced insulin resistance. Subjects or patients with insulin resistance can be administered a polymer-conjugated hyaluronan-degrading enzyme, such as a hyaluronidase, for example a PH20 (e.g. PEG PH20).

The methods and uses provided herein are for treating subjects that typically exhibit symptom(s) associated with insulin resistance. In particular, the methods herein can be used to treat obese patients, who generally exhibit signs and symptoms of insulin resistance. Generally, prior to treatment, patients are selected that exhibit one or more signs or symptoms associated with insulin resistance. It is within the level of a skilled physician to diagnose insulin resistance. Subjects that have insulin resistance, including obesity-induced insulin resistance, generally exhibit high blood sugar (hyperglycemia), weight gain, fat storage and difficulty losing weight, increased blood triglyceride levels (hyperlipemia) and increased hyperinsulinemia. Insulin resistance also can be associated with one or more other symptoms such as fatigue, inability to focus, intestinal bloating, increased blood pressure, increased pro-inflammatory cytokines, depression and/or increased hunger.

Selection of a subject having insulin resistance for treatment with a polymer-conjugated hyaluronan-degrading enzyme in the methods provided herein can be based on fasting insulin levels, fasting plasma glucose (FPG), glucose tolerance testing (GTT or OGTT), quantification using a hyperinsulinemic euglycemic clamp, an insulin tolerance test (ITT), an insulin sensitivity test (IST), continuous infusion of glucose with model assessment (CIGMA), the homeostatic model Assessment (HOMA) index (Matthews et al. (1985) Diabetologia 28:412-419), the Quantitative insulin sensitivity check index (QUICKI; Katz et al. (2000) J. Clin. Endocrinol. Metab. 85:2402-2410), McAuley's index (McAuley et al. (2001) Diabetes Care 24:460-464), Matsuda index (Matsuda and DeFronzo (1999) Diabetes Care 22:1462-1470), Belfiore index (Belfiore et al. (1998) Mol Gen Metab. 63:134-141), Cederholm index (Cederholm and Wibell (1990) Diabetes Res. Clin. Pract. 10:167-175), Gutt index (Gutt et al. (2000) Diabetes Res. Clin. Pract. 47:177-184), Avignon index (Avignon et al. (1999) Int. J. Obes. Relat. Metab. Disord. 23:512-517) or Stumvoll index (Stumvoll et al. (2001) Diabetes Care 24:796-797). Such techniques are well known to one of skill in the art (see, e.g. Borai et al. (2011) BMC Med. Res. Methodol. 11:158; Muniyappa et al. (2008) AJP: Endo. Metab. 294:E15-E26; Ascaso et al. (2003) Diabetes Care 26:3320-5; Radikova (2003) Endocrine Reg. 37:189-194).

In particular examples herein, subjects exhibiting insulin resistance are selected using a hyperinsulinemic euglycemic clamp during which serum glucose levels are “clamped” at a normal fasting concentration by providing a continuous infusion of insulin and simultaneously infusing a variable glucose. The rate of the glucose infusion is varied based on rapid feedback provided by frequently sampled serum glucose. One of skill in the art is familiar with hyperinsulinemic euglycemic clamp. In an exemplary example of the test, insulin is infused generally at a rate of 10-120 mU/m²/min and a glucose preparation (e.g. 20 to 50% glucose) is infused to maintain blood sugar levels between 5 and 5.5 mmol/L. The blood glucose is monitored so that a steady fasting glucose can be maintained. A glucose infusion rate (GINF or GIR) can be determined, which is the amount of glucose necessary to compensate for an increased insulin level without causing hypoglycemia or hyperglycemia. Blood sugar levels are monitored during the course of infusion, and GINF is typically determined during the last 30 minutes of the 2 hour infusion test as steady state values are generally obtained. A glucose disposal rate (GDR) also can be measured, which is derived from the GINF normalized to total body weight (mg/kg per minute). For example, subjects having insulin resistance can be selected for treatment with a polymer-conjugated hyaluronan-degrading enzyme in the methods herein to reduce or ameliorate insulin resistance based on a hyperinsulinemic euglycemic clamp test in which insulin is infused generally at a rate of about or approximately or at least 40 mU/m²/min, which is equivalent to about 1 mU/min per kg body wt. In such examples, subject are treated that have a GDR of less than 7.5 mg/kg per minute, and generally less than 7.0 mg/kg per minute, 6.5 mg/kg per minute, 6.0 mg/kg per minute, 5.5 mg/kg per minute, 5.0 mg/kg per minute, 4.5 mg/kg per minute, 4.0 mg/kg per minute, 3.5 mg/kg per minute, 3.0 mg/kg per minute or lower.

Subjects can be selected for treatment with a polymer-conjugated hyaluronan-degrading enzyme in the methods herein based on fasting plasma glucose (FPG) levels. Generally, blood glucose is measured in a subject who has not eaten for at least or about or approximately 8 hours. For example, provided herein is a method of treating, ameliorating or reducing insulin resistance in a subject by administering a hyaluronan-degrading enzyme conjugated to a polymer where the subject is selected based on a FPG of greater than normal, i.e. exhibits hyperglycemia. For example, subjects are treated that exhibit a FPG of greater than or approximately 100 mg/dL, 110 mg/dL, 120 mg/dL, 125 mg/dL, 130 mg/dL, 135 mg/dL, 140 mg/dL or higher.

Subjects can be selected for treatment with a polymer-conjugated hyaluronan-degrading enzyme in the methods herein based on fasting serum insulin levels. For example, provided herein is a method of treating, ameliorating or reducing insulin resistance in a subject by administering a hyaluronan-degrading enzyme conjugated to a polymer where the subject is selected based on a fasting serum insulin level of greater than normal, i.e. exhibits hyperinsulinemia. For example, subjects are treated that exhibit a fasting serum insulin of greater than or approximately 10 Units/mL, 15 Units/mL, 20 Units/mL, 25 Units/mL, 30 Units/mL, 35 Units/mL, 40 Units/mL or higher.

Subjects can be selected for treatment with a polymer-conjugated hyaluronan-degrading enzyme in the methods herein using an insulin sensitivity test (IST).

Subjects are infused with a defined glucose load and a fixed-rate infusion of insulin over approximately 3 hours. For example, insulin and 20% glucose can be infused at 32 and 267 mg/m²/min, respectively. A steady-state plasma glucose level (SSPG) is assessed after suppressing endogenous insulin and glucose secretion with octreotide (Sandostatin) or somatostatin. Blood glucose is monitored during the test, and values averaged during the last 30 minutes to determine the SSPG. In examples of method provided herein of treating, ameliorating or reducing insulin resistance, a hyaluronan-degrading enzyme conjugated to a polymer is administered to a subject having a fasting glucose concentration greater than 150 mg/dL, and generally greater than 160 mg/dL, 170 mg/dL, 180 mg/dL or greater.

Subjects can be selected for treatment with a polymer-conjugated hyaluronan-degrading enzyme in the methods herein using an insulin tolerance test (Akinmokum et al. (1992) Diabet. Med. 9:432-437). ITT is a test that indirectly measures insulin-stimulated uptake of glucose into skeletal muscle. Following a fast, subjects are given an intravenous bolus of regular insulin (e.g. 0.1 to 0.5 U/kg), and the decline in serum glucose is measured by sampling the blood following the bolus (e.g. within 3 to 30 minutes, such as 3 to 15 minutes). Rate constant for ITT (KITT) can be calculated using the formula KITT (%/min)=0.693/t(½), where t(½) is calculated from the slope of plasma glucose concentration during the sampling period (e.g. 3-15 minutes) after administration of intravenous insulin. Lower insulin-sensitivity index (Kitt) scores mean higher degrees of insulin resistance. A Kitt value below 2.5%/min is indicative of insulin resistance. For examples, in the methods herein of administering a polymer-conjugated hyaluronan-degrading enzyme to a subject for treating or ameliorating insulin resistance in a subject, subjects are selected that exhibit have a Kitt value of less than 2.5%/min, such as less than 2.2%/min, 2.0%/min, 1.8%/min, 1.7%/min, 1.6%/min, 1.5%/min, 1.4%/min, 1.3%/min, 1.2%/min, 1.1%/min, 1.0%/min or less.

Subjects or patients having insulin resistance also can be selected for treatment with a polymer-conjugated hyaluronan-degrading enzyme using a glucose tolerance test (GTT), which is a test assessing blood glucose levels approximately 2 hours after a fasting subject is dosed with glucose. The test can be performed by infusing glucose intravenously (IVGTT; e.g. about 0.3 g/kg of a 50% glucose) or administering an oral dose of glucose (OGTT; e.g. about 75 grams to 100 grams). Glucose (and sometimes insulin) can be measured at various intervals over 2 to 4 hours. World Health Guidelines (WHO) mandate that after 2 hours after the dose, blood glucose (glycemia) less than 7.8 mmol/L (140 mg/dL) is considered normal, while glycemia of greater than 7.8 mmol/L (140 mg/dL) is indicative of an impaired level. For examples, in the methods herein of administering a polymer-conjugated hyaluronan-degrading enzyme to a subject for treating or ameliorating insulin resistance in a subject, subjects are selected that exhibit glycemia of greater than 7.8 mmol/L, and generally greater than 8.0 mmol/L, 9.0 mmol/L, 10.0 mmol/L, 11.0 mmol/L, 12.0 mmol/L or greater. In other examples, in the methods herein, subjects are treated that exhibit glycemia of greater than 140 mg/dL, 150 mg/dL, 160 mg/dL, 170 mg/dL, 180 mg/dL, 190 mg/dL, 200 mg/dL or greater.

In another example, subjects or patients having insulin resistance also can be selected for treatment with a polymer-conjugated hyaluronan-degrading enzyme using a frequency sampled intravenous glucose tolerance test (FSIVGTT), in which an intravenous glucose bolus (e.g. about 0.3 g/kg) is administered followed by a 5 minute insulin infusion 20 minutes later. Blood samples are tested for glucose every two minutes for the first 20 minutes and samples are tested for glucose and insulin levels at later times (e.g. at or about 22, 24, 26, 28, 30, 33, 36, 40, 50, 60, 70, 80, 100, 120, 140, 160 and 180 minutes). In some examples of the test, tolbutamide can be given instead of insulin in the modified FSIVGTT to stimulate endogenous insulin secretion at this time. The data are then subjected to minimal model analysis using the computer program MINMOD to generate an index of insulin sensitivity (S₁; see, e.g. Muniyappa et al. (2008) AJP Endo. Metab. 294:E15-E26). The “TISPAG” software can be used to provide S₁ as wells as glucose effectiveness (available from the Department of Physiology, University of Montpellier). In some examples, a simplified procedure as above can be used, whereby only values of blood glucose for each subject are used for the calculations with a concomitant modification in the software to modify the input for insulin values by replacement with a fixed set of values (see, e.g. Perez-Martin et al. (2002) Horm. Metab. Res. 34:102-106). A lower S₁ value is indicative of insulin resistance. For example, in the methods herein of administering a polymer-conjugated hyaluronan-degrading enzyme to a subject for treating or ameliorating insulin resistance in a subject, subjects are selected that exhibit an S₁ (min₋₁/(μU/ml)·10⁻⁴) value of less than 5.0, and generally less than 4.5, 4.0, 3.5, 3.0, 2.5, 2.4, 2.3, 2.2, 2.1, 2.0, 1.9, 1.8, 1.7, 1.6, 1.5 or less.

In further examples herein, subjects for treatment with a polymer-conjugated hyaluronan-degrading enzyme are selected based on the homeostatic model assessment (HOMA) or HOMA2 index of insulin resistance (see, e.g. Matthews et al. (1985) Diabetologia 28:412-9; Rudenski et al. (1991) Metabolism 40:908-917; Wallace et al. (2004) Diabetes Care 27:1487-95; Levy et al. (1998) Diabetes Care 21:2191-2). The model is based on the presence of elevated fasting glucose levels and the presence of compensatory elevated fasting insulin levels by increased secretion of insulin by β cells, which correlate to the degree of insulin resistance. The model estimates beta cell function (%13) and insulin sensitivity (% S) from simultaneously measured fasting plasma glucose and fasting plasma RIA insulin values (or fasting specific insulin or C-peptide values). An insulin resistance score (HOMA-IR) can be computed with the formula: fasting plasma glucose (mmol/l) times fasting serum insulin (mU/1) divided by 22.5 or insulin (μU/ml)×glucose (mg/dL)/405. Low HOMA-IR values indicate high insulin sensitivity, whereas high HOMA-IR values indicate insulin resistance. Generally, computer-based or calculator-based programs are used to provide a HOMA score. Such models generally are calibrated to give % β and % S values of 100% in normal subjects. A calculator is publicly available and can be downloaded to calculate HOMA values (see e.g. www.dtu.ox.ac.uk/homacalculator/download.php). In examples of the methods herein of administering a polymer-conjugated hyaluronan-degrading enzyme to a subject for treating or ameliorating insulin resistance in a subject, subjects are selected that exhibit a HOMA score of greater than 2.2 or 2.3, and generally greater than 2.5, 2.6, 2.7, 2.8, 2.9, 3.0, 3.5, 4.0, 5.0, 6.0 or greater. For example, a HOMA score of between or about between 2.5 to 2.6 shows insulin resistance.

In a related example, subjects for treatment with a polymer-conjugated hyaluronan-degrading enzyme are selected based on quantitative insulin sensitivity check index (QUICKI) is related to FSIVGTT and is defined by the formula: 1/(log glucose [mg/dL]+log insulin [μU/ml]). In examples of the methods herein of administering a polymer-conjugated hyaluronan-degrading enzyme to a subject for treating or ameliorating insulin resistance in a subject, subjects are selected that exhibit a QUICKI index of less than 0.360, and generally less than 0.359, 0.358, 0.357, 0.355, 0.350, 0.345, 0.340, 0.335, 0.330, 0.325, 0.320, 0.315, 0.310, 0.305, 0.300 or less.

In a further example, subjects for treatment with a polymer-conjugated hyaluronan-degrading enzyme are selected based on the McAuley (McA) index (McAuley et al. (2001) Diabetes Care 24:460-464), in which scores are determined based on weighted combinations of fasting insulin, BMI and fasting triglycerides using the following formula: McA=exp (2.63−0.28 ln insulin [μU/ml]−0.31 ln triglycerides [mM/ml]). In examples of the methods herein of administering a polymer-conjugated hyaluronan-degrading enzyme to a subject for treating or ameliorating insulin resistance in a subject, subjects are selected that exhibit a McA index of less than 6.5, and generally less than 6.3, 6.0, 5.9, 5.8, 5.7, 5.6, 5.5, 5.0, 4.5, 4.0, 3.5, 3.0 or less.

Other methods for assessing insulin resistance, including for selecting subjects having insulin resistance, include any methods known to one of skill in the art. Exemplary of such methods are described in Section F. For example, subjects can be selected based on waist circumference, weight or serum triglycerides, serum HDL cholesterol, blood pressure and other routine clinical measurements (Stern et al. (2005) Diabetes 54:333-339). For example, commonly used criteria and cut-offs for insulin resistance include, for example, a waist circumference in males of greater than 102 cm and in females of greater than 88 cm; BMI of greater than 30 kg/m²; a triglyceride to HDL ratio of greater than 3.0, apolipoprotein B levels greater than 1.36 g/L; blood pressure greater than 135/85 mmHg; and C-reactive protein greater than 3.0 mg/L.

It is understood that the cut-off values provided herein above correlating the existence of insulin resistance are exemplary only. It is understood that values above or below the indicated values can be indicative of insulin resistance. Variation in the levels can occur due to differences in age, gender, nationality, weight and extent and severity of the condition. It is within the level of a skilled physician to identify patients that exhibit features of insulin resistance. Generally, more than one of any of the above parameters, if present, are indicative of the presence of insulin resistance.

b. Conditions and Diseases Associated with Insulin Resistance

Insulin resistance is associated with or can cause or contribute to the pathogenesis of diverse syndromes. In particular, insulin resistance is a component of the “metabolic syndrome,” which is a cluster of debilitating health care problems. Metabolic syndrome is often characterized by any of a number of metabolic disorders or risk factors, which are generally considered to most typify metabolic syndrome when more than one of these factors are present in a single individual. The factors include: central obesity (disproportionate fat tissue in and around the abdomen), atherogenic dyslipidemia (these include a family of blood fat disorders including, e.g. high triglycerides, low HDL cholesterol, and high LDL cholesterol that can foster plaque buildups in the vascular system, including artery walls), high blood pressure (130/85 mmHg or higher), insulin resistance or glucose intolerance (the inability to properly use insulin or blood sugar), a chronic prothrombotic state (e.g. characterized by high fibrinogen or plasminogen activator inhibitor [−1] levels in the blood), and a chronic proinflammatory state (e.g. characterized by higher than normal levels of high-sensitivity C-reactive protein in the blood). People with metabolic syndrome are at increased risk of coronary heart disease, other diseases related to plaque buildups in artery walls (e.g. stroke and peripheral vascular disease) and Type 2 Diabetes.

Exemplary of diseases and conditions associated with insulin resistance include, but are not limited to, type 2 diabetes mellitus, obesity, arterial hypertension, dyslipidemia, atherosclerosis, coronary disease, polycystic ovary syndrome. Generally, insulin resistance precedes the development of these syndromes, and can occur before the manifestation of such diseases or conditions. Hence, insulin resistance is an early marker of such diseases and conditions. By administering a polymer-conjugated hyaluronan-degrading enzyme to reduce or ameliorate insulin resistance, the methods herein effect prevention or amelioration of diseases or conditions that are associated with insulin resistance.

Hence, the methods herein of administering a polymer-conjugated hyaluronan-degrading enzyme can be used to prevent, treat or ameliorate diseases and conditions that are associated with insulin resistance, including, but not limited to, type 2 diabetes mellitus, obesity, arterial hypertension, dyslipidemia, atherosclerosis, coronary disease, polycystic ovary syndrome. Exemplary of diseases or conditions associated with insulin resistance are described below. The therapeutic uses described below are exemplary and do not limit the applications of the methods described herein.

i. Type 2 Diabetes

Provided herein is a method of preventing or ameliorating type 2 diabetes mellitus (T2DM) by administering a polymer-conjugated hyaluronan-degrading enzyme to a subject as described herein. Diabetes is defined as a state in which carbohydrate and lipid metabolism are improperly regulated by insulin. T2DM is the most common form and results from a combination of defects in insulin secretion and insulin action. Individuals with insulin resistance indicate a pre-diabetic state, are thus predisposed to developing T2DM, as the failure of insulin to stimulate glucose uptake in muscle appears to be a primary defect. For example, prediabetes or insulin resistance is characterized by FPG levels generally less than 125 mg/dL (e.g. 100 to 125 mg/dL), while T2DM is diagnosed if plasma glucose levels are greater than 126 mg/dL. In other examples, a GTT (e.g. IVGTT or OGTT) can be used to diagnose subjects, whereby a 2-hour plasma glucose result of greater than 140 mg/dL, and generally 140 mg/dL to 199 mg/dL indicates diabetes or insulin resistance, while a 2-hour plasma glucose result of greater than 200 mg/dL is indicative of T2DM.

Pancreatic β cells normally compensate for the insulin resistant state by increasing insulin secretion, which further aggravates insulin resistance. Eventually, the β cells can no longer compensate adequately for insulin resistance by secreting more insulin and thus fail to respond appropriately to glucose. This ultimately leads to the deterioration of glucose homeostasis and the development of glucose intolerance, the inability to properly dispose of glucose. Approximately 5%-10% of glucose-intolerant patients per year progress to T2DM, which continues to worsen as insulin resistance increases. Adipose and liver cells generate more fatty acids, the liver produces more glucose in an unregulated fashion, and the β cells undergo progressive decompensation, resulting in the late stages of the disease, where high doses of exogenous insulin may be required.

ii. Cardiovascular Disease Provided herein is a method of preventing or ameliorating cardiovascular disease (CVD) by administering a polymer-conjugated hyaluronan-degrading enzyme to a subject as described herein. CVD refers to several types of heart conditions, and is a major cause of morbidity and mortality in much of the world. The most common type is coronary artery disease, which can cause heart attacks, angina, heart failure, and arrhythmias. Other related conditions include stroke, aortic aneurysm and dissection, cardiomyopathy, peripheral arterial disease, and rheumatic heart disease. Hyperinsulinemia has been shown to be a predictor of CVD in insulin resistant individuals (McFarlane et al. (2001) J. Clin. Endocrinol. Metabol. 86(2):713-718; Ginsberg (2000) J. Clin. Invest. 106:453-458). Insulin resistance is associated with risk factors of CVD including an atherogenic lipid profile, endothelial dysfunction and increased risk of thrombosis. In particular, hyperinsulinemia is associated with hypertension, coronary artery disease (CAD), hypercoagulability and atherosclerosis.

In particular, persons with insulin resistance generally exhibit dyslipidemia. Dyslipidemia develops due to the inability of insulin-resistant fat cells to store triglycerides (TG). There is generally increased intracellular hydrolysis of TGs and release of fatty acids into the circulation. The effect is hypertriglyceridemia and an increase in the production of triglycerides and triglyceride-rich low density lipoproteins (LDL). Decreased levels of high-density lipoprotein (HDL) also exist in persons with insulin resistance. The dyslipidemia can contribute to the development of atherosclerotic plaques.

In addition to the dyslipidemia, insulin resistance also causes hypertension that can be a separate risk factor to heart disease. Abnormalities in vasodilation and blood flow are possible links between hypertension and insulin resistance. This has been proposed to be due to defects in insulin-stimulated nitrogen oxide (NO) by endothelial cells. Also, hyperinsulinemia can result in increased reabsorption of sodium and water by kidney tubular cells, which can be associated with hypertension. In addition, hyperinsulinemia also can result in hyperuricemia, which can be associated with cardiovascular disease.

Subjects with insulin resistance also exhibit an increase in factors involved in clotting that are associated with a risk of cardiovascular disease. Such factors include, but are not limited to, fibrinogen, factor VII and plasminogen activator inhibitor 1 (PAI-1). The dysregulation of these factors is due to hyperlipidemia and hyperinsulinemia that occurs in insulin resistance. Impaired insulin action on endothelial or vascular smooth muscle cells that occurs in insulin resistance also can have direct effects on artery wall thickening.

Iii. Polycystic Ovary Syndrome (PCOS)

Provided herein is a method of preventing or ameliorating polycystic ovary syndrome (PCOS) by administering a polymer-conjugated hyaluronan-degrading enzyme to a subject as described herein. PCOS is characterized by anovulation (irregular or absent menstrual periods) and hyperandrogenism with elevated serum testosterone and androstenedione. Due to the high levels of androgenic hormones, PCOS results in increased LH levels, anovulation, amenorrhea, recurrent pregnancy loss and infertility Hyperinsulinemia that accompanies insulin resistance produces the hyperandrogenism by increasing ovarian androgen production, such as testosterone, and decreasing the serum sex hormone binding globulin concentration.

2. Dosage and Dosage Regimen (Frequency and Cycle of Administration)

The polymer-conjugated hyaluronan-degrading enzyme, for example a hyaluronidase such as a PH20 (e.g. PEGPH20) is administered in an amount sufficient to exert a therapeutically useful effect. It is within the level of one of skill in the art to determine the precise amounts of polymer-conjugated hyaluronan-degrading enzyme to be administered to a subject. It is understood that the precise dosage and duration of treatment is a function of the particular subject (e.g. age of the individual treated, the weight of the individual), the route of administration, the disease or condition being treated, the severity or extent of the disease or condition being treated and other similar factors. Based on such factors, the precise dosage, and frequence of dosage, can be determined empirically using known testing protocols or by extrapolation from in vivo or in vitro test data and/or can be determined from known dosing regimes of the particular agent. For example, the amount of a polymer-conjugated hyaluronan-degrading enzyme, such as a soluble hyaluronidase, for example a PH20 (e.g. PEGPH20), to be administered for the treatment of a disease or condition, for example insulin resistance or a metabolic syndrome associated with insulin resistance, can be determined by standard clinical techniques. In addition, in vitro assays and animal models can be employed to help identify optimal dosage ranges.

Generally, dosage regimens are chosen to limit toxicity. It should be noted that the attending physician would know how to and when to terminate, interrupt or adjust therapy to lower dosage due to toxicity, or bone marrow, liver or kidney or other tissue dysfunctions. Conversely, the attending physician would also know how to and when to adjust treatment to higher levels if the clinical response is not adequate (precluding toxic side effects). It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope thereof.

For example, the polymer-conjugated hyaluronan-degrading enzyme, such as a hyaluronidase for example a PH20 (e.g. PEGPH20), is administered in a therapeutically effective amount to degrade, cleave or remove skeletal muscle-associated hyaluronan. Exemplary dosage range is at or about 50 Units to 50,000,000 Units of a hyaluronan-degrading enzyme conjugated to a polymer, or a functionally equivalent amount of another hyaluronan-degrading enzyme conjugated to a polymer. It is understood herein that a unit of activity is normalized to a standard activity, for example, an activity as measured in a microturbidity assay assaying hyaluronidase activity.

Thus, for example, a hyaluronan-degrading enzyme, such as a hyaluronidase for example a PH20, conjugated to polymer, for example, a PEG, can be administered at or about 10 to 50,000,000 Units, 10 to 40,000,000 Units, 10 to 36,000,000 Units, 10 to 12,000,000 Units, 10 to 1,200,000 Units, 10 to 1,000,000 Units, 10 to 500,000 Units, 100 to 100,000 Units, 500 to 50,000 Units, 1000 to 10,000 Units, 5000 to 7500 Units, 5000 Units to 50,000 Units, or 1,000 to 10,000 Units. Generally, a polymer-conjugated hyaluronan-degrading enzyme is administered to a subject in an amount that is between or about between 0.01 μg/kg to 25 mg/kg, such as 0.0005 mg/kg (0.5 μg/kg) to 25 mg/kg, 0.5 μg/kg to 10 mg/kg (320,000 U/kg), 0.02 mg/kg to 1.5 mg/kg, 0.01 μg/kg to 15 μg/kg, 0.05 mg/kg to 10 mg/kg, 0.75 μg/kg to 7.5 μg/kg or 1.0 μg/kg to 3.0 μg/kg. The polymer-conjugated hyaluronan-degrading enzyme can be administered, for example, at a dosage of at least or about at least 0.0005 mg/kg (of the subject), 0.0006 mg/kg, 0.0007 mg/kg, 0.0008 mg/kg, 0.0009 mg/kg, 0.001 mg/kg, 0.0016 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.016 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.15 mg/kg, 0.2 mg/kg, 0.25 mg/kg, 0.30 mg/kg, 0.35 mg/kg, 0.40 mg/kg, 0.45 mg/kg, 0.5 mg/kg, 0.55 mg·kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, or more is administered, to an average adult human subject, typically weighing about 70 kg to 75 kg.

A polymer-conjugated hyaluronan-degrading enzyme, such as a PEGylated hyaluronidase (e.g. PEGPH20), provided herein can be administered at between or about between 1 Unit/kg to 800,000 Units/kg, such as 10 to 800,000 Units/kg, 10 to 750,000 Units/kg, 10 to 700,000 Units/kg, 10 to 650,000 Units/kg, 10 to 600,000 Units/kg, 10 to 550,000 Units/kg, 10 to 500,000 Units/kg, 10 to 450,000 Units/kg, 10 to 400,000 Units/kg, 10 to 350,000 Units/kg, 10 to 320,000 Units/kg, 10 to 300,000 Units/kg, 10 to 280,000 Units/kg, 10 to 260,000 Units/kg, 10 to 240,000 Units/kg, 10 to 220,000 Units/kg, 10 to 200,000 Units/kg, 10 to 180,000 Units/kg, 10 to 160,000 Units/kg, 10 to 140,000 Units/kg, 10 to 120,000 Units/kg, 10 to 100,000 Units/kg, 10 to 80,000 Units/kg, 10 to 70,000 Units/kg, 10 to 60,000 Units/kg, 10 to 50,000 Units/kg, 10 to 40,000 Units/kg, 10 to 30,000 Units/kg, 10 to 20,000 Units/kg, 10 to 15,000 Units/kg, 10 to 12,800 Units/kg, 10 to 10,000 Units/kg, 10 to 9,000 Units/kg, 10 to 8,000 Units/kg, 10 to 7,000 Units/kg, 10 to 6,000 Units/kg, 10 to 5,000 Units/kg, 10 to 4,000 Units/kg, 10 to 3,000 Units/kg, 10 to 2,000 Units/kg, 10 to 1,000 Units/kg, 10 to 900 Units/kg, 10 to 800 Units/kg, 10 to 700 Units/kg, 10 to 500 Units/kg, 10 to 400 Units/kg, 10 to 300 Units/kg, 10 to 200 Units/kg, 10 to 100 Units/kg, 16 to 600,000 Units/kg, 16 to 500,000 Units/kg, 16 to 400,000 Units/kg, 16 to 350,000 Units/kg, 16 to 320,000 Units/kg, 16 to 160,000 Units/kg, 16 to 80,000 Units/kg, 16 to 40,000 Units/kg, 16 to 20,000 Units/kg, 16 to 16,000 Units/kg, 16 to 12,800 Units/kg, 16 to 10,000 Units/kg, 16 to 5,000 Units/kg, 16 to 4,000 Units/kg, 16 to 3,000 Units/kg, 16 to 2,000 Units/kg, 16 to 1,000 Units/kg, 16 to 900 Units/kg, 16 to 800 Units/kg, 16 to 700 Units/kg, 16 to 500 Units/kg, 16 to 400 Units/kg, 16 to 300 Units/kg, 16 to 200 Units/kg, 16 to 100 Units/kg, 160 to 12,800 Units/kg, 160 to 8,000 Units/kg, 160 to 6,000 Units/kg, 160 to 4,000 Units/kg, 160 to 2,000 Units/kg, 160 to 1,000 Units/kg, 160 to 500 Units/kg, 500 to 5000 Units/kg, 1000 to 100,000 Units/kg or 1000 to 10,000 Units/kg, of the mass of the subject to whom it is administered. In some examples, a hyaluronan-degrading enzyme, such as a polymer-conjugated hyaluronan-degrading enzyme, such as a PEGylated hyaluronidase (e.g. PEGPH20) can be administered at or about 1 Unit/kg to 1000 Units/kg, 1 Units/kg to 500 Units/kg or 10 Units/kg to 50 Units/kg.

Generally, where the specific activity of the PEGylated hyaluronidase is or is about 10,000 U/mg to 80,000 U/mg, such as 20,000 U/mg to 60,000 U/mg or 18,000 U/mg to 45,000 U/mg, generally at or about 1 Units/kg (U/kg), 2 U/kg, 3 U/kg, 4 U/kg, 5 U/kg, 6 U/kg, 7 U/kg, 8 U/kg, 8 U/kg 10 U/kg, 16 U/kg, 32 U/kg, 64 U/kg, 100 U/kg, 200 U/kg, 300 U/kg, 400 U/kg, 500 U/kg, 600 U/kg, 700 U/kg, 800 U/kg, 900 U/kg, 1,000 U/kg, 2,000 U/kg, 3,000 U/kg, 4,000 U/kg, 5,000 U/kg, 6,000 U/kg, 7,000 U/kg, 8,000 U/kg, 9,000 U/kg, 10,000 U/kg, 12,800 U/kg, 20,000 U/kg, 32,000 U/kg, 40,000 U/kg, 50,000 U/kg, 60,000 U/kg, 70,000 U/kg, 80,000 U/kg, 90,000 U/kg, 100,000 U/kg, 120,000 U/kg, 140,000 U/kg, 160,000 U/kg, 180,000 U/kg, 200,000 U/kg, 220,000 U/kg, 240,000 U/kg, 260,000 U/kg, 280,000 U/kg, 300,000 U/kg, 320,000 U/kg, 350,000 U/kg, 400,000 U/kg, 450,000 U/kg, 500,000 U/kg, 550,000 U/kg, 600,000 U/kg, 650,000 U/kg, 700,000 U/kg, 750,000 U/kg, 800,000 U/kg or more, per mass of the subject, is administered. For example, 60,000 U; 70,000 U; 80,000 U; 90,000 U; 100,000 U; 200,000 U; 300,000 U; 400,000 U; 500,000 U; 600,000 U; 700,000 U; 800,000 U; 900,000 U; 1,000,000 U; 1,500,000 U; 2,000,000 U; 2,500,000 U; 3,000,000 U; 3,500,000 U; 4,000,000 U or more is administered.

The polymer-conjugated hyaluronan-degrading enzyme can be administered intravenously, subcutaneously, intratumorally, intradermally, orally or by other routes of administration

The frequency and timing of administration, and the dosage amounts, can be administered periodically over a cycle of administration to maintain a continuous and/or long term effect of the hyaluronan-degrading enzyme for a desired length of time. Hence, chronic reduction of skeletal muscle-associated hyaluronan can be achieved. The compositions can be administered hourly, daily, weekly, monthly, yearly or once. The length of time of the cycle of administration can be empirically determined, and is dependent on the disease to be treated, the severity of the disease, the particular patient, and other considerations within the level of skill of the treating physician. The length of time of treatment with a composition provided herein can be one week, two weeks, one months, several months, one year, several years or more. The dosages can be divided into a plurality of dosages over the course of a cycle of administration during the course of treatment. For example, the frequency of administration of a polymer-conjugated hyaluronan-degrading enzyme, such as a hyaluronidase enzyme, for example a PH20 (e.g. PEGPH20) in a cycle of administration can be once weekly, twice weekly, three times a week, four times a week, five times a week, six times a week or seven times a week during a cycle of administration. Exemplary of a dosage regime herein is administration of a polymer-conjugated hyaluronan-degrading enzyme to a subject twice a week for four weeks (e.g. 28 days), which cycle is repeated at the discretion of the treating physician. If disease symptoms persist in the absence of discontinued treatment, treatment can be continued for an additional length of time. Over the course of treatment, evidence of disease and/or treatment-related toxicity or side effects can be monitored.

In addition, the cycle of administration can be tailored to add periods of discontinued treatment in order to provide a rest period from exposure to the agent. The length of time for the discontinuation of treatment can be for a predetermined time or can be empirically determined depending on how the patient is responding or depending on observed side effects. For example, the treatment can be discontinued for one week, two weeks, one month or several months. Generally, the period of discontinued treatment is built into a cycle of dosing regime for a patient.

For example, an exemplary dosing regime is a treatment cycle or cycle of administration of 28 days. The agent, such as the polymer-conjugated hyaluronan-degrading enzyme, can be administered once weekly, twice weekly or three times a week. For example, the agent can be administered for the first 3 weeks, once weekly, twice weekly or three times a week, followed by a one week without dosing. Thus, for example, a patient can be dosed with the agent twice weekly on days 1, 4, 8, 11, 15 and 18 (or days 0, 3, 6, 9, 12 and 15 or other variations), followed by a one-week of discontinued treat, over the course of the 28-day cycle. In another example, a patient can be dosed with the agent once weekly on days 1, 8 and 16 (or other variation of once weekly dosing, followed by a one-week of discontinued treatment over the course of the 28-day cycle. In another example, the agent can be administered for the full 28 days once weekly, twice weekly or three times a week. Thus, for example, a patient can be dosed with the agent twice weekly on days 1, 4, 8, 11, 15, 18, 21 and 24 (or days 0, 4, 7, 11, 14, 18, 21 or 25 or other variations of twice weekly dosing) for the full 28 day cycle. In other examples, a patient can be dosed with the agent once weekly on days 1, 8, 16 or 24 (or other variations of a once weekly dosing) for the full 28 day cycle. It is understood that the above description is for exemplification purposes only and that variations of the above can be employed.

It is within the level of one of skill in the art to determine the precise cycle of administration and dosing schedule. As noted above, the cycle of administration can be for any desired length of time. For example, the cycle of administration can be continued over the course of several months or years. Hence, the 28-day cycle of administration can be repeated for any length of time. It is within the level of skill of the treating physician to adopt a cycle of administration and dosing regime that meets the needs of the patient depending on personal considerations specific to the patient and disease to be treated.

3. Selecting Insulin Resistant Subjects Having Increased Skeletal Muscle Hyaluronan

The methods herein include steps for selecting subjects for treatment that have increased or elevated hyaluronan (HA) levels associated with skeletal muscle. Exemplary assays for detecting hyaluronan include direct and indirect assays for measuring HA level and/or relative HA levels in a skeletal muscle sample from a subject, assays for analyzing effects of hyaluronan-degrading enzymes on a skeletal muscle sample from the subject, and other parameters associated with hyaluronan levels such as hyaluronidase expression or activity, interstitial fluid pressure, vascular volume and water content. In general, any known assay for detection of proteins or nucleic acids in samples from subjects, or for assessing the effects of treatment on cells/tissues in vitro can be used. One of skill in the art is familiar with methods of detecting hyaluronan, which include, but are not limited to, immunohistochemistry methods or ELISA methods.

Subjects selected for treatment in the methods provided herein include subjects having elevated, aberrant or accumulated levels of hyaluronan in skeletal muscle compared to subjects not having the disease or condition (e.g. insulin resistance) or compared to normal tissues or samples that do not have elevated, aberrant or accumulated expression of HA. Any sample or tissue from a subject can be tested and compared to a normal sample or tissue. For example, hyaluronan levels can be measured from any source such as from a tissue (e.g. by biopsy), tumor, cells, or from blood, serum, urine or other body fluids. Generally, skeletal muscle samples are collected by biopsy and hyaluronan levels assessed and compared. In such examples, the marker is elevated or increased if the detected HA levels are elevated compared to normal subjects, e.g. elevated from between at or about 2-fold to at or about 9-fold, e.g. increased at least or at or about 2, 3, 4, 5, 6, 7, 8 or 9-fold elevation compared to normal subject.

In one example, the step for detecting hyaluronan markers is performed prior to treating a subject, for example, to determine whether the subject has elevated or increased hyaluronan levels that will be amenable to treatment with a hyaluronan-degrading enzyme as described herein. In this example, if the marker is detected (e.g. if it is determined that a skeletal muscle tissue sample or a fluid sample from the patient contains elevated hyaluronan expression or is responsive to hyaluronan-degrading enzyme), a treatment step is performed, where a polymer-conjugated hyaluronan-degrading enzyme is administered to the subject.

In another example, the step for detecting markers is performed after treating a subject, or during the course of treatment of the subject, (e.g. treatment with a polymer-conjugated hyaluronan-degrading enzyme (e.g. soluble modified hyaluronidase such as a PH20, for example a PEGPH20), for example, to determine whether the treatment with the hyaluronan-degrading enzyme is having an effect on treating the disease or condition. In one such example, the marker is not detected or is detected at an amount or relative level that is decreased compared to the amount/level prior to treatment, or compared to another sample, treatment can be discontinued or modified (i.e. dosage decreased or frequency of administration decreased). In another such example, if the marker is detected at the same level as prior to treatment or another sample, treatment can be continued or another treatment option can be selected. In a further example, if the marker is detected at an increased level as prior to treatment or another sample, treatment can be continued, treatment can be modified (e.g. dosage increased or frequency of administration increased), or another treatment, such as a combination therapy, is initiated.

The assays to detect hyaluronan markers include assays to measure amount (e.g. relative amount) of hyaluronan and/or hyaluronidase expression in a tissue (e.g. skeletal muscle such as gastrocnemius), cell and/or body fluid of a subject. Included amongst such assays are those that can detect HA expression, a hyaluronan synthase (e.g. HAS2) expression, and the presence of hyaluronan-degrading enzymes, such as hyaluronidases, for example, in samples from the subject.

Assays to detect protein and nucleic acid levels are well known in the art and can be used in the methods herein to measure hyaluronan, hyaluronan synthase or other protein and/or nucleic acid expression. Such assays include, but are not limited to, ELISA, SDS-PAGE, Western Blot, PCR, RT-PCR, immunohistochemistry, histology and flow cytometry. For example, a sample from a subject, such as a tissue sample (e.g. a biopsy of a skeletal muscle from a patient or animal model), a fluid (e.g. blood, urine, plasma, saliva or other sample), a cell or cellular sample, or extract, or other sample, can be stained with anti-HA antibodies or other HA binding protein (e.g. HABP), for example, using histological staining, such as immunohistochemistry (IHC) of fixed or frozen tissue sections, to determine the presence and extent of hyaluronan in the tissue or sample, or immunofluorescent cellular staining, pull-down assays, and flow cytometry. For example, tissue extracts (from diseased subjects and control or reference samples) can be coated on microwell plates, followed by incubation (e.g. 16 hours, room temperature) with a labeled (e.g. biotinylated) HA binding protein, washed and the HA-binding protein bound to the wells quantified using an avidin-biotin detection agent substrate. Such methods are well known in the art. In another example, the sample, e.g. biopsy, can be assayed by RT-PCR to assess the amount of HA mRNA.

In a further example, hyaluronan synthase 2 (HAS2) production and/or expression by cells in vitro, ex vivo or in vivo also can be assayed by, for example, ELISA, SDS-PAGE, Western Blot, PCR, RT-PCR, immunohistochemistry, histology or flow cytometry. In another example, the amount of hyaluronidase activity in a sample from the subject is determined, such as in the blood or plasma such as with a turbidity assay.

In another example, a cell or other tissue from a patient is isolated, e.g. a isolated muscle or skeletal muscle cell, and used in a study to determine whether the cell or tissue is responsive to treatment with the hyaluronan-degrading enzyme in vitro, for example, an assay to assess signaling, proliferation, metabolic uptake or growth of the cell or tissue. For example, skeletal muscle cells from a subject can be seeded on surface, such as an extracellular matrix or protein mixture, such as the mixture sold under the trade name Matrigel® (BD Biosciences). In this example, the hyaluronan-associated marker is the sensitivity of the cell or tissue to administration of hyaluronan-degrading enzyme. In this example, if any property, such as proliferation, growth or survival of the cells, is inhibited or blocked by addition of hyaluronan-degrading enzyme, it is determined that the subject may be amenable to treatment with hyaluronan-degrading enzyme containing compositions.

In addition to assays for determining hyaluronan expression levels, other assays can be used to select a subject for treatment, and/or to assess treatment efficacy and/or duration. For example, interstitial fluid pressure (IFP) can be assessed by methods and techniques well known to one of skill in the art. In one example, IFP can be measured using an appropriate probe or instrument. For example, a transducer-tipped catheter can be used to measure the IFP in muscle tissues or other tissues of interest. The catheter is passed through the inner bore of a surgical needle, which is then inserted into the muscle. The needle is withdrawn while the catheter is held in position. The IFP (mmHg) can then be measured using an appropriate data acquisition unit (Ozerdem et al. (2005) Microvasc. Res. 70:116-120). Other methods to measure IFP include the wick-in-needle method (Fadnes et al. (1977) Microvasc. Res. 14:27-36).

Skeletal muscle vascular volume changes also can be measured by methods and techniques well known to one of skill in the art. For example, vascular volume can be measured by, for example, using ultrasound imaging. This method employs hyper-echoic microbubbles to provide the strong ultrasound wave reflections that are detected. The microbubbles, when injected, such as intravenously, into a subject or animal model, become trapped in the vascular space due to their size. Assays to assess tissue water content, such as muscle tissue water content, also are known in the art. For example, samples from a muscle can be harvested, blotted, weighed and snap frozen before being lyophilized. The water weight is then reported as the tissue wet weight to dry (i.e. lyophilized) weight ratio.

For any of the detection methods, the marker (e.g. HA expression, responsiveness to hyaluronan-degrading enzyme, HA-synthase expression or hyaluronidase activity) typically is compared to a control sample, such that detection of the marker typically includes determining that the readout is elevated or reduced compared to the control sample. It is understood that the particular change, e.g. increase in or decrease in HA, is dependent on the assay used. For example, in an ELISA, the fold increase or decrease in absorbance at a particular wavelength or in quantity of protein (e.g. as determined by using a standard curve) can be expressed relative to a control. In a PCR assay, such as RT-PCR, expression levels are compared to control expression levels (e.g. expressed as fold change) using methods known to those of skill in the art, such as using standards. For example, when the amount of hyaluronan in a sample from a subject is being tested, tissue (e.g. skeletal muscle), cell (e.g. skeletal muscle cell), or fluid (e.g. blood) is determined to have increased or elevated HA if the amount of HA in the tissue, cell or fluid is elevated at or about 0.5-fold, 1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 20-fold, or more, compared to the control sample.

For example, the control sample can be another tissue, cell or body fluid, such as a normal tissue, cell or body fluid, for example, a tissue, cell or body fluid that is analogous to the sample being tested, but isolated from a different subject, such as a subject that is normal (i.e. does not exhibit insulin resistance, or does not have the type of disease or condition that the subject being tested has), or an analogous tissue from another subject that has a similar disease or condition, but whose disease is not as severe and/or expresses relatively less hyaluronan. Generally, for purposes of selecting patients exhibiting increased or elevated HA, the control sample is a fluid, tissue, extract (e.g. cellular or nuclear extract), nucleic acid or peptide preparation, cell line, biopsy, standard or other sample, with a known amount or relative amount of HA. For example, human skeletal muscle cell lines are known and available and can serve as control or reference samples. Exemplary of such cells lines include characterized lots of primary human skeletal muscle derived cells (HDkMDC; e.g. obtained from Cook Myosite, Pittsburgh, Pa.; Cell Applications, San Diego, Calif.; or other similar commercial provider or vendor or source). Cell lines also can be used (e.g. Caviedes et al. (1992) Biochimica et Biophysica Acta (BBA)—Molecular Cell Research, 1134:247-255). In other examples, typically for monitoring the course of treatment, the sample is a skeletal muscle tissue or cell, and is compared to a reference sample obtained from the same subject prior to treatment or at an earlier point in treatment.

4. Monitoring Subjects for Reduced Insulin Resistance

The polymer-conjugated hyaluronan-degrading enzymes provided herein reduce, lessen or ameliorate insulin resistance, and thereby also can prevent or ameliorate diseases and conditions associated with insulin resistance, including diabetes and cardiovascular disease. The insulin resistance or insulin sensitivity of the subject can be monitored over time to assess whether a reduction in insulin resistance has been achieved over the course of therapy with a hyaluronan-degrading enzyme, such as a hyaluronan-degrading enzyme provided herein conjugated to a polymer. Typically, the hyaluronan-degrading enzyme is administered until the insulin resistance is reduced by at least 5%, 10%, 20%, 30%, 40%, 50%, 60%, 70%, 80% or more.

In the methods herein, in order to assess a reduction in insulin resistance, the subjects can be monitored during the course of treatment for features or parameters associated with insulin resistance. Typically, one or more signs or symptoms from among hyperglycemia, body fat, weight, hyperlipemia and/or hyperinsulinemia can be monitored. An effective dosage amount or dosage regime is generally one that ameliorates, reduces, lessens or prevents one or more of hyperglycemia, body fat, weight, hyperlipemia and/or hyperinsulinemia in the subject compared to a control or reference. The control or reference can be a standard or can be a sample from a normal or healthy subject. In other instances, the control or reference can be a sample from the same subject prior to treatment or a sample taken during an earlier time of the dosage cycle (e.g. taken following the previous administration of a hyaluronan-degrading enzyme).

For example, any of the methods or tests described above to select subject having insulin resistance can be used to assess insulin resistance during the course of treatment. Hence, in the methods herein, the treatment can be monitored to confirm that the treatment is effective. i.e. it prevents, ameliorates or reduces the incidence of insulin resistance and/or the development of an associated metabolic syndrome. For example, therapeutic effectiveness of administered polymer-conjugated hyaluronan-degrading enzyme can be monitored using any of the above tests or methods, including but not limited to GTT, a hyperinsulinemic euglycemic clamp, ITT, IST, CIGMA, HOMA or QUICKI. It is understood that the method used for determining if a patient has insulin resistance and is a candidate for treatment with a polymer-conjugated hyaluronan-degrading enzyme does not need to be the same method that is used to monitor the treatment following administration of the polymer-hyaluronan-degrading enzyme. It is known to one of skill in the art that the tests and methods for assessing insulin resistance generally correlate and can be compared. Hence, in some examples, the test or method used to assess insulin resistance and select subjects for treatment is different than the test or method used to monitor treatment with an administered polymer-conjugated hyaluronan-degrading enzyme. In other instances, the test or method used to assess insulin resistance and select subjects for treatment is the same as the test or method used to monitor treatment with an administered polymer-conjugated hyaluronan-degrading enzyme.

Generally, in any of the tests, treatment of a polymer-conjugated hyaluronan-degrading enzyme is therapeutically effective if the index or parameter of insulin resistance is improved by greater than 5%, 10%, 20%, 30%, 40%, 50%, 60%, 70%, 80% or more compared to the same index or parameter prior to treatment or prior to an earlier time of the dosage cycle (e.g. taken following the previous administration of a hyaluronan-degrading enzyme).

In one example, a hyperinsulinemic euglycemic clamp can be effected on a subject during the course of treatment, and GDR can be monitored during the course of treatment to assess the therapeutic efficacy of an administered polymer-conjugated hyaluronan-degrading enzyme. Treatment can be continued until GDR is increased by greater than 5%, 10%, 20%, 30%, 40%, 50%, 60%, 70%, 80% or more. Generally, insulin resistance is ameliorated when GDR levels are greater than 7.0 mg/kg per minutes or higher, and generally greater than 7.5 mg/kg per minute, 7.6 mg/kg/min, 7.7 mg/kg per minute, 7.8 mg/kg per minute, 7.9 mg/kg per minute, 8.0 mg/kg per minute, 8.5 mg/kg per minute, 9.0 mg/kg per minute or higher.

In examples herein, fasting serum insulin level can be monitored during the course of treatment to confirm that the hyperinsulinemic is reduced to assess the therapeutic efficacy of an administered polymer-conjugated hyaluronan-degrading enzyme. Treatment can be continued until fast-insulin is reduced by greater than 5%, 10%, 20%, 30%, 40%, 50%, 60%, 70%, 80% or more. Generally, insulin resistance is ameliorated when fasting insulin levels are less than 12 Units/mL, 11 Units/mL, 10 Units/mL, 9 Units/mL, 8 Units/mL, 7 Units/mL, 6 Units/mL, 5 U/mL or less.

In other examples, an insulin sensitivity test can be used during the course of treatment to monitor treatment, and the SSPG can be monitored during the course of treatment to assess the therapeutic efficacy of an administered polymer-conjugated hyaluronan-degrading enzyme. In examples herein, treatment can be continued until SSPG is decreased by greater than 5%, 10%, 20%, 30%, 40%, 50%, 60%, 70%, 80% or more. Generally, insulin resistance is ameliorated when SSPG levels are less than 150 mg/dL, 140 mg/dL, 130 mg/dL, 120 mg/dL or less.

In an additional example, an insulin tolerance test can be used during the course of treatment to monitor treatment, and Kitt values obtained to assess the therapeutic efficacy of an administered polymer-conjugated hyaluronan-degrading enzyme. Treatment can be continued Kitt value is increased by greater than 5%, 10%, 20%, 30%, 40%, 50%, 60%, 70%, 80% or more. Generally, insulin resistance is ameliorated when Kitt values are at least or about at least 2.4%/min, such as at least 2.5%/min, 2.6%/min, 2.7%/min, 2.8%/min, 2.9%/min, 3.0%/min, 3.2%/min, 3.4%/min, 3.6%/min, 3.8%/min, 4.0%/min, 5.0%/min or greater.

In a further example, a glucose tolerance test can be used to monitor blood glucose during the course of treatment to assess the therapeutic efficacy of an administered polymer-conjugated hyaluronan-degrading enzyme. Treatment can be continued until blood glucose is reduced by greater than 5%, 10%, 20%, 30%, 40%, 50%, 60%, 70%, 80% or more. Generally, insulin resistance is ameliorated when blood glucose levels are within the normal range of less than 7.8 mmol/L or less than 140 mg/dL or less.

In an additional example, a homeostatic model assessment (HOMA) can be made as an index of insulin resistance to assess the therapeutic efficacy of an administered polymer-conjugated hyaluronan-degrading enzyme. Treatment can be continued until HOMA-IR is reduced by greater than 5%, 10%, 20%, 30%, 40%, 50%, 60%, 70%, 80% or more. Generally, insulin resistance is ameliorated when a HOMA score is less than 3.0, such as less than 2.8, 2.6, 2.5, 2.4, 2.3, 2.2, 2.1, 2.0, 1.5, 1.25, 1.0 or less.

In another example, a quantitative insulin sensitivity check index (QUICKI) can be made as determined as an index of insulin resistance to assess the therapeutic efficacy of an administered polymer-conjugated hyaluronan-degrading enzyme. Treatment can be continued until QUICKI is increased by greater than 5%, 10%, 20%, 30%, 40%, 50%, 60%, 70%, 80% or more. Generally, insulin resistance is ameliorated when a QUICKI score is greater than 3.3, such as greater than 3.4, 3.5, 3.6, 3.7, 3.8, 3.9, 4.0 or greater.

In a further example, a McAuley (McA) index score can be determined as an indicator of insulin resistance to assess the therapeutic efficacy of an administered polymer-conjugated hyaluronan-degrading enzyme. Treatment can be continued until McA is increased by greater than 5%, 10%, 20%, 30%, 40%, 50%, 60%, 70%, 80% or more. Generally, insulin resistance is ameliorated when a McA score is greater than 5.6, such as greater than 5.8, 6.0, 6.2, 6.5, 6.8, 7.0, 7.5, 8.0 or greater.

Other methods for assessing insulin resistance, including for monitoring treatment, include any methods known to one of skill in the art. Exemplary of such methods are described in Section F. For example, subjects can be monitored or evaluated for a change in body fat. Body fat measurements can be determined by a variety of techniques known to one of skill in the art, including, but not limited to determinations of skinfold thickness, bioelectrical impedance, air displacement plethysmography, underwater weighing, DEXA scans, measurement on a scale or calculation of body mass index (BMI). Generally, administered polymer-conjugated hyaluronan-degrading enzymes are effective if body fat is decreased by greater than 5%, 10%, 20%, 30%, 40%, 50%, 60%, 70%, 80% or more. Therapeutic effectiveness of also can be determined by a decrease in weight of the subject as determined by a standard scale. Generally, administered polymer-conjugated hyaluronan-degrading enzymes are effective if weight is decreased by greater than 5%, 10%, 20%, 30%, 40%, 50%, 60%, 70%, 80% or more.

5. Combination Treatments

The compositions provided herein can be used alone or in further combination with other therapies or treatments. The compositions provided herein can be further co-formulated or co-administered together with, prior to, intermittently with, or subsequent to, other therapeutic or pharmacologic agents or treatments, such as lipid-lowering drugs, hypoglycemic drugs, anti-hypertensive agents. In some cases, the use of a polymer-conjugated hyaluronan-degrading enzyme can be used in combination with a diet and/or exercise regime. Such agents also can include one or more agents to ameliorate, reduce or prevent side effects.

A preparation of a second agent or agents or treatment or treatments can be administered at once, or can be divided into a number of smaller doses to be administered at intervals of time. Selected agent/treatment preparations can be administered in one or more doses over the course of a treatment time for example over several hours, days, weeks, or months. In some cases, continuous administration is useful. It is understood that the precise dosage and course of administration depends on the indication and patient's tolerability. Generally, dosing regimes for second agents/treatments herein are known to one of skill in the art.

a. Other Agents or Treatments for Treating Insulin Resistance or Associated Diseases and Conditions

The methods provided herein of administering a polymer-conjugated hyaluronan-degrading enzyme for reducing insulin resistance or preventing or ameliorating diseases or conditions associated with insulin resistance, can further include treatment in combination with other agents or treatments for insulin resistance or the diseases or conditions associated with insulin resistance. For example, the agent or treatment can be one that also manages, ameliorates or reduces insulin resistance. In other examples, the further agent or treatment can be one that focuses on a separate (albeit related in some cases) risk factor or component of a disease or conditions associated with insulin resistance. For example, for treatment of CVD, the hypertension or dyslipidemia can be targeted in combination therapy herein by further treating with a lipid-lowering drugs or anti-hypertensive agent. For the treatment of diabetes, insulin can be administered. The particular additional agent or treatment depends on the particular disease or condition (e.g. CVD vs. T2DM). It is within the level of a skilled physician to select an appropriate combination therapy depending on the particular patient being treated (e.g. age and weight), the extent or severity of disease, the history of the patient and other relevant factors.

For example, the methods herein can include combination treatment effected by weight loss, exercise and/or dietary changes. For example, these dietary changes can include: maintaining a diet that limits carbohydrates to 50 percent or less of total calories; eating foods defined as complex carbohydrates, such as whole grain bread (instead of white), brown rice (instead of white), sugars that are unrefined, increasing fiber consumption by eating legumes (for example, beans), whole grains, fruits and vegetables, reducing intake of red meats and poultry, consumption of “healthy” fats, such as those in olive oil, flaxseed oil and nuts, and/or limiting alcohol intake. In some examples, bariatric surgery can be performed.

An insulin-sensitizing drug can be used in combination therapy with the polymer-conjugated hyaluronan-degrading enzymes herein for treating or reducing insulin resistance. Such agents include, but are not limited to, metformin (Glucophage), rosiglitazone (Avandia), pioglitazone (Actos) and troglitazone (Rezulin), and D-chiro-inositol. Such drugs can be used in combination therapy for ameliorating or reducing insulin resistance, or for preventing or reducing a disease or condition associated with insulin resistance accompanied by dyslipidemia, hyperinsulinemia, hypertension and/or atherosclerosis. Exemplary of such diseases and conditions include, but are not limited to T2DM, CVD and PCOS.

In other examples, for treatment of cardiovascular disease associated with insulin resistance, the methods provided herein of administering a polymer-conjugated hyaluronan-degrading enzyme for reducing insulin resistance or preventing or ameliorating diseases or conditions associated with insulin resistance, can further include treatment with a lipid-lowering drugs or anti-hypertensive agents. For example, treatment of blood pressure, and blood triglyceride levels can be controlled by a variety of available drugs, including but not limited to cholesterol modulating drugs or aspirin therapy. Cholesterol or lipid lowering drugs include, but are not limited to, statins (e.g. atorvastatin (Lipitor®), pravastatin (Pravachol®), lovastatin (Altoprev® and Mevacor®), fluvastatin (Lescol®), simivastatin (Zicor®) and rosuvastatin (Crestor®); resins (e.g. L-Cholest, cholestyramine (Prevalite® and Questran®), colesevelam (Welchol®) and colestipol (Colestid®); cholesterol absorption inhibitors (e.g. ezetimibe (Zetia®), simvastatin and ezetimibe combined (Vytorin®)); fibrates (e.g. fenofibrate (Tricor®), bezabifrate (Bezalip®), gemifibrozil (Lopid®)); and nicotinic acid (e.g. niacin).

Anti-hypertensive agents that can be used in combination therapy herein include, but are not limited to, agents acting on the renin-angiotensin system (e.g. renin inhibitors or Angiotensin II receptor antagonists), adrenergic receptor antagonists (e.g. alpha blockers, beta blockers or combinations thereof), calcium channel blockers (e.g. dihydropyridines or non-dihydropyridine), diuretics, ACE inhibitors, aldosterone antagonists, vasodilators and alpha-2 agonists. Exemplary of such agents include, but are not limited to, bumetanide, ethacrynic acid, furosemide, torsemide, epitizide, hydrochlorothiazide, chlorothiazide, bendroflumethiazide, indapamide, chlorthalidone, metolazone, amiloride, triamterene, spironolactone, atenolol, metoprolol, nadolol, oxprenolol, pindolol, propranolol, timolol, doxazosin, phentolamine, indoramin, phenoxybenzamine, prazosin, terazosin, tolazoline, bucindolol, carvedilol, labetalol, amlodipine, felodipine, isradipine, lercanidipine, nicardipine, nifedipine, nimodipine, nitrendipine, diltiazem, verapamil, captopril, enalapril, fosinopril, lisinopril, perindopril, quinapril, ramipril, trandolapril, benazepril, candesartan, eprosartan, irbesartan, losartan, olmesartan, telmisartan, valsartan, eplerenone, spironolactone, sodium nitroprusside, hydralazine, clonidine, guanabenz, methyldopa, moxonidine, guanethidine, and reserpine. Combination therapy also can include treatment with a conjugated linoleic acid (CLA).

If insulin resistance leads to T2DM, there are many treatments available for this disease. Exemplary combination agents are hypoglycemic drugs. Typically, treatment of T2DM includes insulin treatment. The insulin can be a intermediate insulin, long-acting insulin (e.g. insulin detemir (Levemir®), insulin glargine (Lantus®)) or a rapid-acting insulin analog (e.g. insulin lispro (Humalog®), insulin aspart (NovoLog®), insulin glulisine (Apidra®)). In particular examples, the insulin is a super-fast acting insulin composition (e.g. U.S. Published Patent Appl. No. US20090304665). Since T2DM patients are resistant to insulin, high doses can be required.

Other exemplary agents, for example for treatment of T2DM, include, but are not limited to, agents to augment insulin secretion, such as sulfonylureas; agents that bind to the IGF-1 receptor or insulin receptor (IR), such as IGF-1; agents that improve insulin sensitivity, such as a biguanide (e.g. metformin), a thiazolidinedione (e.g. troglitazone), vandate or vanadium salts and phenyloin. Other agents include, but are not limited to, sitagliptin (Januvia®), saxagliptin (Onglyza®), repaglinide (Prandin®), nateglinide (Starlix®), Exenatide (Byetta®) and liraglutide (Victoza®). In addition, other therapeutic agents that exert hypoglycemic activity include, for example, nutraceuticals or other natural health products including, but not limited to, buckwheat, magnesium supplementation, chromium picolinate, Caiapo (white sweet potato extract), flaxseed extract, American ginseng, oat bran, oat gum and vanadium.

In further examples of insulin resistance associated with polycystic ovary syndrome (PCOS), combination treatments include, but are not limited to, ovulation medications (e.g. clomiphene, letrozole, Follistime, Gonal-F), anti-androgen medications (e.g. birth control pills, spironolactone, flutamide or finasteride), ovarian drilling surgery and in vitro fertilization (IVF)

b. Corticosteroids

The polymer-conjugated hyaluronan-degrading enzyme compositions provided herein can be used alone or in further combination with one or more corticosteroids. A corticosteroid is administered in an amount that is therapeutically effective to ameliorate or reduce one or more adverse effects of administration of a polymer-conjugated hyaluronan-degrading enzymes or other agent, in particular, adverse musculoskeletal effects. A therapeutically effective amount is the dosage sufficient to ameliorate, prevent, eliminate or reduce one or more symptoms or adverse effects. Indicators of improvement or successful pretreatment include determination of the failure to manifest a relevant score on the CTCAE scale or a change in grading or severity on the CTCAE scale.

Corticosteroids are a class of steroid hormones that are produced in the adrenal cortex. Corticosteroids are involved in a wide range of physiologic systems such as stress response, immune response and regulation of inflammation, carbohydrate metabolism, protein catabolism, blood electrolyte levels, and behavior. These include glucocorticoids, which are anti-inflammatory agents with a large number of other functions and mineralocorticoids, which control salt and water balance primarily through action on the kidneys.

Glucocorticoids are a class of steroid hormones, e.g. corticosteroids, that bind to the glucocorticoid receptor. Glucocorticoids cause their effects by binding to the glucocorticoid receptor. The activated glucocorticoid complex in turn up-regulates the expression of anti-inflammatory proteins in the nucleus and represses the expression of pro-inflammatory proteins in the cytosol by preventing the translocation of other transcription factors from the cytosol into the nucleus.

Generally, any corticosteroid, e.g. glucocorticoid, can be used in the methods or combinations provided herein. The glucocorticoids include synthetic and non-synthetic glucocorticoids. Exemplary glucocorticoids include, but are not limited to: alclomethasones, algestones, beclomethasones (e.g. beclomethasone dipropionate), betamethasones (e.g. betamethasone 17-valerate, betamethasone sodium acetate, betamethasone sodium phosphate, betamethasone valerate), budesonides, clobetasols (e.g. clobetasol propionate), clobetasones, clocortolones (e.g. clocortolone pivalate), cloprednols, corticosterones, cortisones and hydrocortisones (e.g. hydrocortisone acetate), cortivazols, deflazacorts, desonides, desoximethasones, dexamethasones (e.g. dexamethasone 21-phosphate, dexamethasone acetate, dexamethasone sodium phosphate), diflorasones (e.g. diflorasone diacetate), diflucortolones, difluprednates, enoxolones, fluazacorts, flucloronides, fludrocortisones (e.g. fludrocortisone acetate), flumethasones (e.g. flumethasone pivalate), flunisolides, fluocinolones (e.g. fluocinolone acetonide), fluocinonides, fluocortins, fluocortolones, fluorometholones (e.g. fluorometholone acetate), fluperolones (e.g. fluperolone acetate), fluprednidenes, fluprednisolones, flurandrenolides, fluticasones (e.g. fluticasone propionate), formocortals, halcinonides, halobetasols, halometasones, halopredones, hydrocortamates, hydrocortisones (e.g. hydrocortisone 21-butyrate, hydrocortisone aceponate, hydrocortisone acetate, hydrocortisone buteprate, hydrocortisone butyrate, hydrocortisone cypionate, hydrocortisone hemisuccinate, hydrocortisone probutate, hydrocortisone sodium phosphate, hydrocortisone sodium succinate, hydrocortisone valerate), loteprednol etabonate, mazipredones, medrysones, meprednisones, methylprednisolones (methylprednisolone aceponate, methylprednisolone acetate, methylprednisolone hemisuccinate, methylprednisolone sodium succinate), mometasones (e.g. mometasone furoate), paramethasones (e.g. paramethasone acetate), prednicarbates, prednisolones (e.g. prednisolone 25-diethylaminoacetate, prednisolone sodium phosphate, prednisolone 21-hemisuccinate, prednisolone acetate; prednisolone farnesylate, prednisolone hemisuccinate, prednisolone-21 (beta-D-glucuronide), prednisolone metasulphobenzoate, prednisolone steaglate, prednisolone tebutate, prednisolone tetrahydrophthalate), prednisones, prednivals, prednylidenes, rimexolones, tixocortols, triamcinolones (e.g. triamcinolone acetonide, triamcinolone benetonide, triamcinolone hexacetonide, triamcinolone acetonide 21-palmitate, triamcinolone diacetate). These glucocorticoids and the salts thereof are discussed in detail, for example, in Remington's Pharmaceutical Sciences, A. Osol, ed., Mack Pub. Co., Easton, Pa. (16th ed. 1980).

In some examples, the glucocorticoid is selected from among cortisones, dexamethasones, hydrocortisones, methylprednisolones, prednisolones and prednisones. In a particular example, the glucocorticoid is dexamethasone.

The corticosteroid is provided in a therapeutically effective dose. Therapeutically effective concentration can be determined empirically by testing in known in vitro or in vivo (e.g. animal model) systems. For example, the amount of a selected corticosteroid to be administered to ameliorate the adverse effects can be determined by standard clinical techniques. In addition, animal models can be employed to help identify optimal dosage ranges. The precise dosage, which can be determined empirically, can depend on the particular therapeutic preparation, the regime and dosing schedule, the route of administration and the seriousness of the disease.

The concentration of a selected therapeutic agent in the composition depends on absorption, inactivation and excretion rates, the physicochemical characteristics, the dosage schedule, and amount administered as well as other factors known to those of skill in the art. For example, it is understood that the precise dosage and duration of treatment is a function of the disease or condition, the tissue being treated, the patient or subject and the particular polymer-conjugated hyaluronan-degrading enzyme, including amount and dosage regime. The dose of the corticosteroid also can vary depending on the age and health of the patient, the polymer-conjugated hyaluronan-degrading enzyme dosing (e.g. PEGylated hyaluronan-degrading enzyme dosing), potency of the corticosteroid, and the route of administration. For example, it is to be noted that concentrations and dosage values will vary with the therapeutic dose and dosage regime of the hyaluronan-degrading enzyme. Additionally, the corticosteroid can be administered daily, weekly, or monthly or over longer periods of time in order to achieve the desired results. The particular dosage volume can vary and is dependent on the dosage regime, frequency of administration and the desired rate of administration. It is to be noted that concentrations and dosage values can also vary with the age of the individual treated.

The precise dosage and duration of treatment can be determined empirically using known testing protocols or by extrapolation from in vivo or in vitro test data. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope thereof. Generally, dosage regimens are chosen to limit toxicity, and herein are chosen to ameliorate adverse side effects. It should be noted that the attending physician would know how to and when to terminate, interrupt or adjust therapy to lower dosage due to toxicity, or bone marrow, liver or kidney or other tissue dysfunctions. Conversely, the attending physician would also know how to and when to adjust treatment to higher levels if the clinical response is not adequate (precluding toxic side effects). Administration of a therapeutic agent should not exceed the maximum dosage levels established by the United States Food and Drug Administration or published in the Physician's Desk Reference.

Generally, the dose of corticosteroid administered is dependent upon the specific corticosteroid, as a difference in potency exists between different corticosteroids (see Table 4 below). The corticosteroid, or glucocorticoid, for example dexamethasone, can be given orally (tablets, liquid or liquid concentrate) PO, intravenously (IV) or intramuscularly. The corticosteroid is typically administered as a bolus, but many be administered over a period of time, as long as the dose is effective to ameliorate one or more side effects associated with administration of the polymer-conjugated hyaluronan-degrading enzyme, for example, a PEGylated hyaluronidase.

TABLE 4
Glucocorticoid administration

| Glucocorticoid (route) | Equivalent Potency (mg) |
|---|---|
| Hydrocortisone (IV or PO) | 20 |
| Prednisone | 5 |
| Prednisolone (IV or PO) | 5 |
| Methylprednisolone sodium succinate (IV) | 4 |
| Dexamethasone (IV or PO) | 0.5-0.75 |

The corticosteroid can be administered in any amount that is effective to ameliorate one or more side effects associated with administration of the polymer-conjugated hyaluronan-degrading enzyme. Thus, the corticosteroid, e.g. glucocorticoid, can be administered, for example, at an amount between at or about 0.1 and 100 mgs, per dose, 0.1 to 80 mgs, 0.1 to 60 mgs, 0.1 to 40 mgs, 0.1 to 30 mgs, 0.1 to 20 mgs, 0.1 to 15 mgs, 0.1 to 10 mgs, 0.1 to 5 mgs, 0.2 to 40 mgs, 0.2 to 30 mgs, 0.2 to 20 mgs, 0.2 to 15 mgs, 0.2 to 10 mgs, 0.2 to 5 mgs, 0.4 to 40 mgs, 0.4 to 30 mgs, 0.4 to 20 mgs, 0.4 to 15 mgs, 0.4 to 10 mgs, 0.4 to 5 mgs, 0.4 to 4 mgs, 1 to 20 mgs, 1 to 15 mgs or 1 to 10 mgs, to a 70 kg adult human subject. Typically, the corticosteroid, such as a glucocorticoid is administered at an amount between at or about 0.4 and 20 mgs, for example, at or about or at least 0.4 mgs, 0.5 mgs, 0.6 mgs, 0.7 mgs, 0.75 mgs, 0.8 mgs, 0.9 mgs, 1 mg, 2 mgs, 3 mgs, 4 mgs, 5 mgs, 6 mgs, 7 mgs, 8 mgs, 9 mgs, 10 mgs, 11 mgs, 12 mgs, 13 mgs, 14 mgs, 15 mgs, 16 mgs, 17 mgs, 18 mgs, 19 mgs or 20 mgs per dose, to an average adult human subject.

The corticosteroid can be administered, for example, at a dosage of at or about or at least 0.001 mg/kg (of the subject), 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.015 mg/kg, 0.02 mg/kg, 0.025 mg/kg, 0.03 mg/kg, 0.035 mg/kg, 0.04 mg/kg, 0.045 mg/kg, 0.05 mg/kg, 0.055 mg/kg, 0.06 mg/kg, 0.065 mg/kg, 0.07 mg/kg, 0.075 mg/kg, 0.08 mg/kg, 0.085 mg/kg, 0.09 mg/kg, 0.095 mg/kg, 0.1 mg/kg, 0.15 mg/kg, 0.2 mg/kg, 0.25 mg/kg, 0.30 mg/kg, 0.35 mg/kg, 0.40 mg/kg, 0.45 mg/kg, 0.50 mg/kg, 0.55 mg/kg, 0.60 mg/kg, 0.65 mg/kg, 0.70 mg/kg, 0.75 mg/kg, 0.80 mg/kg, 0.85 mg/kg, 0.90 mg/kg, 0.95 mg/kg, 1 mg/kg, 1.05 mg/kg, 1.1 mg/kg, 1.15 mg/kg, 1.20 mg/kg, 1.25 mg/kg, 1.3 mg/kg, 1.35 mg/kg or 1.4 mg/kg, to an average adult human subject, typically weighing about 70 kg to 75 kg.

The dosage administered administration can vary as long as administration of the corticosteroid ameliorates one or more adverse side effects associated with administration of the polymer-conjugated hyaluronan-degrading enzyme. In one example, the dosage of glucocorticoid, for example, dexamethasone, is adminstered in successively lower dosages per treatment cycle. Hence, in such treatment regimes, the dose of corticosteroid is tapered. For example, dexamethasone is administered prior to administration of a polymer-conjugated hyaluronan-degrading enzyme, at an initial dose of 4 mg, and upon each successive administration of the hyaluronan-dergrading enzyme, the dexamethasone dose is lowered, such that the dose is 3 mg for the next administration of the hyaluronan-degrading enzyme, e.g. PEGylated hyaluronidase, then 2 mg per administration of anti-hyaluronan agent, e.g. PEGylated hyaluronidase, and then 1 mg per administration of anti-hyaluronan agent, e.g. PEGylated hyaluronidase. Any dose is contemplated as long as the dose of the corticosteroid is effective to reduce one or more side effects associated with administration of the hyaluronan-degrading enzyme, e.g. a PEGylated hyaluronidase.

Time of administration can vary as long as administration of the corticosteroid ameliorates one or more adverse side effects associated with administration of the hyaluronan-degrading enzyme, such as a PEGylated hyaluronidase. The corticosteroid can be administered sequentially, intermittently, at the same time or in the same composition as hyaluronan-degrading enzyme, e.g. PEGylated hyaluronan-degrading enzyme. For example, the corticosteroid can be administered before, during, simultaneously with, or after administration of the hyaluronan-degrading enzyme, e.g. PEGylated hyaluronidase. In another example, the corticosteroid and hyaluronan-degrading enzyme, e.g. PEGylated hyaluronidase are administered intermittently. Generally, the corticosteroid is administered prior to administration of the hyaluronan-degrading enzyme, e.g. PEGylated hyaluronidase. For example, the corticosteroid, e.g. glucocorticoid, such as dexamethasone, can be administered at or about 0.5 minutes, 1 minute, 2 minutes, 3 minutes, 4 minutes, 5 minutes, 6 minutes, 7 minutes, 8 minutes, 9 minutes, 10 minutes, 15 minutes, 20 minutes, 25 minutes, 30 minutes, 45 minutes, 1 hour, 2 hours, 3 hours, 4 hours, 5 hours, 6 hours, 7 hours, 8 hours, 9 hours, 10 hours, 11 hours, 12 hours, 18 hours, 24 hours, 36 hours or more prior to administration of the anti-hyaluronan agent, for example a PEGylated hyaluronan-degrading enzyme.

In some examples, the corticosteroid is administered at the same time as administration of the polymer-conjugated hyaluronan-degrading enzyme, for example a PEGylated hyaluronan-degrading enzyme. In this example, the corticosteroid can be administered together with, or separately from, the hyaluronan-degrading enzyme, e.g. a PEGylated hyaluronidase. Typically, the corticosteroid is administered separately from the hyaluronan-degrading enzyme, for example a PEGylated hyaluronan-degrading enzyme. In other examples, the corticosteroid is administered at or about 0.5 minutes, 1 minute, 2 minutes, 3 minutes, 4 minutes, 5 minutes, 6 minutes, 7 minutes, 8 minutes, 9 minutes, 10 minutes, 15 minutes, 20 minutes, 25 minutes, 30 minutes, 45 minutes, 1 hour, 2 hours, 3 hours, 4 hours, 5 hours, 6 hours, 7 hours, 8 hours, 9 hours, 10 hours, 11 hours, 12 hours, 18 hours, 24 hours, 36 hours or more after administration of the hyaluronan-degrading enzyme, for example a PEGylated hyaluronan-degrading enzyme.

In one example, the corticosteroid is administered prior to administration of a polymer-conjugated hyaluronan-degrading enzyme, for example a PEGylated hyaluronidase. For example, the corticosteroid, e.g. glucocorticoid, for example, dexamethasone, is administered 1 hour prior to the administration of the hyaluronan-degrading enzyme, e.g. a PEGylated hyaluronidase. In another example, the corticosteroid is administered 5 minutes before the administration of the hyaluronan-degrading enzyme, e.g. a PEGylated hyaluronan-degrading enzyme. In another example, the corticosteroid is administered both prior to and after the administration of hyaluronan-degrading enzyme, e.g. a PEGylated hyaluronidase. In this example, the corticosteroid, such as dexamethasone, is administered one to five minutes immediately before administration of the polymer-conjugated hyaluronan-degrading enzyme, e.g. a PEGylated hyaluronan-degrading enzyme and eight hours after administration of the anti-hyaluronan agent, e.g. a PEGylated hyaluronan-degrading enzyme. In another example, a corticosteroid, such as dexamethasone, is administered one hour before administration of the hyaluronan-degrading enzyme, e.g. a PEGylated hyaluronan-degrading enzyme and eight to twelve hours after administration of the anti-hyaluronan agent, e.g. a PEGylated hyaluronan-degrading enzyme.

Any dosing regime is contemplated as long as the time of dosing of the corticosteroid ameliorates the one or more side effects associated with administration of the hyaluronan-degrading enzyme, for example a PEGylated hyaluronidase. In addition, the dose or dosing regime of corticosteroid is one that does not interfere or reduce the therapeutic effect of the polymer-conjugated hyaluronan-degrading enzyme in the methods provided herein, including in reducing or ameliorating insulin resistance or preventing or ameliorating a metabolic syndrome associated with insulin resistance.

### H. EXAMPLES

The following examples are included for illustrative purposes only and are not intended to limit the scope of the invention.

### Example 1

### Measurement of Hyaluronic Acid Content in Skeletal Muscle of Obese Mice

High-fat (HF) diet-induced obese (D10) mice were used as a model of skeletal muscle insulin resistance (Surwit et al. (1988) Diabetes 37:1163-1167). The effect of a high fat diet on the hyaluronic acid (HA) content of the extracellular matrix (ECM) of the skeletal muscle of male C57/BL6J mice was assessed. Male C57/BL6J mice were purchased from the Jackson Laboratory (Bar Harbor, Me.) and housed under temperature and humidity-controlled environmental conditions with a 12 hour light/dark cycle. All animal procedures were approved by the Vanderbilt Animal Care and Use Committee.

Mice were fed either a regular rodent chow (Purina) diet or a high fat (HF) diet containing 60% calories as fat (F3282, BioServ, Frenchtown, N.J.) in order to induce obesity. After maintaining the diet for 20 weeks, the mice were sacrificed following a 5-hour fast after the last feeding. The gastrocnemius were collected and the HA content was assessed by immunohistochemistry.

Immunohistochemistry was performed to assess HA content. Briefly, paraffin-embedded tissue sections (5 μm) were incubated for overnight at 4° C. with a primary antibody, biotinylated HA-binding protein (B-HABP, Catalog No. 400763-1A, Seikagaku, East Falmouth, Mass.), as a probe for HA detection and digital quantification. After washing to remove primary reagent, samples were incubated with a streptavidin-horseradish peroxidase (HRP) solution for 30 minutes, lightly counterstained with Mayer's hematoxylin, and detected with 3,3′-diaminobenzidine (DAB) using EnVision+HRP/DAB System (DakoCytomation, Carpenteria, Calif.) to produce localized, visible staining according to the manufacturer's instructions. The images were captured using a Q-Imaging Micropublisher camera (Surrey, BC, Canada) mounted on an Olympus upright microscope (Tokyo, Japan) and immunostaining was quantified by ImageJ Software (developed by the National Institute of Health). HA expression was measured by the integrated intensity of staining. The data were normalized to HA content in mice that were fed regular rodent chow.

The immunohistochemical stains showed a 2-fold increase in the HA content in the skeletal muscle of the mice fed for 20 weeks with a HF diet (n=6; p<0.05) as compared to the mice that were fed regular rodent chow (n=5), indicating that induced obesity increases the HA content in the ECM of insulin resistant mouse skeletal muscle.

### Example 2

### PH20-Expressing Cell Lines and Expression

### A. Generation of an Initial Soluble PH20-Expressing Cell Line

Chinese Hamster Ovary (CHO) cells were transfected with the HZ24 plasmid (set forth in SEQ ID NO:52). The HZ24 plasmid vector for expression of soluble

PH20 contains a pCI vector backbone (Promega), DNA encoding amino acids 1-482 of human PH20 hyaluronidase (SEQ ID NO:49), an internal ribosomal entry site (IRES) from the ECMV virus (Clontech), and the mouse dihydrofolate reductase (DHFR) gene. The pCI vector backbone also includes DNA encoding the Beta-lactamase resistance gene (AmpR), an f1 origin of replication, a Cytomegalovirus immediate-early enhancer/promoter region (CMV), a chimeric intron, and an SV40 late polyadenylation signal (SV40). The DNA encoding the soluble PH20 construct contains an NheI site and a Kozak consensus sequence prior to the DNA encoding the methionine at amino acid position 1 of the native 35 amino acid signal sequence of human PH20, and a stop codon following the DNA encoding the tyrosine corresponding to amino acid position 482 of the human PH20 hyaluronidase set forth in SEQ ID NO:1, followed by a BamHI restriction site. The construct pCI-PH20-IRES-DHFR-SV40pa (HZ24), therefore, results in an mRNA species driven by the CMV promoter that encodes amino acids 1-482 of human PH20 (set forth in SEQ ID NO:3) and amino acids 1-186 of mouse dihydrofolate reductase (set forth in SEQ ID NO:53) separated by the internal ribosomal entry site (IRES).

Non-transfected CHO cells growing in GIBCO Modified CD-CHO media for DHFR(−) cells, supplemented with 4 mM glutamine and 18 ml/L Plurionic F68/L (Gibco), were seeded at 0.5×10⁶ cells/mL in a shaker flask in preparation for transfection. Cells were grown at 37° C. in 5% CO₂ in a humidified incubator, shaking at 120 rpm. Exponentially growing non-transfected CHO cells were tested for viability prior to transfection.

Sixty million viable cells of the non-transfected CHO cell culture were pelleted and re-suspended to a density of 2×10⁷ cells in 0.7 mL of 2×transfection buffer (2×HeBS: 40 mM Hepes, pH 7.0, 274 mM NaCl, 10 mM KCl, 1.4 mM Na₂HPO₄, 12 mM dextrose). To each aliquot of re-suspended cells, 0.09 mL (250 μg) of the linear HZ24 plasmid (linearized by overnight digestion with Cla I (New England Biolabs) was added, and the cell/DNA solutions were transferred into 0.4 cm gap BTX (Gentronics) electroporation cuvettes at room temperature. A negative control electroporation was performed with no plasmid DNA mixed with the cells. The cell/plasmid mixes were electroporated with a capacitor discharge of either 330 V and 960 μF or 350 V and 960 μF.

The cells were removed from the cuvettes after electroporation and transferred into 5 mL of Modified CD-CHO media for DHFR(−) cells, supplemented with 4 mM glutamine and 18 mL/L Plurionic F68/L (Gibco), and allowed to grow in a well of a 6-well tissue culture plate without selection for 2 days at 37° C. in 5% CO₂ in a humidified incubator.

Two days post-electroporation, 0.5 mL of tissue culture media was removed from each well and tested for the presence of hyaluronidase activity using the microturbidity assay described in Example 4. Cells expressing the highest levels of hyaluronidase activity were collected from the tissue culture well, counted and diluted to 1×10⁴ to 2×10⁴ viable cells per mL. A 0.1 mL aliquot of the cell suspension was transferred to each well of five, 96-well round bottom tissue culture plates. One hundred microliters of CD-CHO media (GIBCO) containing 4 mM GlutaMAX™-1 supplement (GIBCO™, Invitrogen Corporation) and without hypoxanthine and thymidine supplements were added to the wells containing cells (final volume 0.2 mL).

Ten clones were identified from the 5 plates grown without methotrexate. Six of these HZ24 clones were expanded in culture and transferred into shaker flasks as single cell suspensions. Clones 3D3, 3E5, 2G8, 2D9, 1E11, and 4D10 were plated into 96-well round bottom tissue culture plates using a two-dimensional infinite dilution strategy in which cells were diluted 1:2 down the plate, and 1:3 across the plate, starting at 5000 cells in the top left hand well. Diluted clones were grown in a background of 500 non-transfected DG44 CHO cells per well, to provide necessary growth factors for the initial days in culture. Ten plates were made per subclone, with 5 plates containing 50 nM methotrexate and 5 plates without methotrexate.

Clone 3D3 produced 24 visual subclones (13 from the no methotrexate treatment, and 11 from the 50 nM methotrexate treatment). Significant hyaluronidase activity was measured in the supernatants from 8 of the 24 subclones (>50 Units/mL), and these 8 subclones were expanded into T-25 tissue culture flasks. Clones isolated from the methotrexate treatment protocol were expanded in the presence of 50 nM methotrexate. Clone 3D35M was further expanded in 500 nM methotrexate in shaker flasks and gave rise to clones producing in excess of 1,000 Units/mL hyaluronidase activity (clone 3D35M; or Gen1 3D35M). A master cell bank (MCB) of the 3D35M cells was then prepared.

B. Generation of a Second Generation Cell Line Expressing Soluble rHuPH20

The Gen1 3D35M cell line described in Example 2A was adapted to higher methotrexate levels to produce generation 2 (Gen2) clones. 3D35M cells were seeded from established methotrexate-containing cultures into CD CHO medium containing 4 mM GlutaMAX-1™ and 1.0 μM methotrexate. The cells were adapted to a higher methotrexate level by growing and passaging the cells 9 times over a period of 46 days in a 37° C., 7% CO₂-humidified incubator. The amplified population of cells was cloned out by limiting dilution in 96-well tissue culture plates containing medium with 2.0 μM methotrexate. After approximately 4 weeks, clones were identified and clone 3E10B was selected for expansion. 3EIOB cells were grown in CD CHO medium containing 4 mM GlutaMAX-1™ and 2.0 μM methotrexate for 20 passages. A master cell bank (MCB) of the 3E10B cell line was created and frozen and used for subsequent studies.

Amplification of the cell line continued by culturing 3EIOB cells in CD CHO medium containing 4 mM GlutaMAX-1™ and 4.0 μM methotrexate. After the 12^{th} passage, cells were frozen in vials as a research cell bank (RCB). One vial of the RCB was thawed and cultured in medium containing 8.0 μM methotrexate. After 5 days, the methotrexate concentration in the medium was increased to 16.0 μM, then 20.0 μM 18 days later. Cells from the 8^{th} passage in medium containing 20.0 μM methotrexate were cloned out by limiting dilution in 96-well tissue culture plates containing CD CHO medium containing 4 mM GlutaMAX-1™ and 20.0 μM methotrexate. Clones were identified 5-6 weeks later and clone 2B2 was selected for expansion in medium containing 20.0 μM methotrexate. After the 11th passage, 2B2 cells were frozen in vials as a research cell bank (RCB).

The resultant 2B2 cells are dihydrofolate reductase deficient (dhfr-) DG44 CHO cells that express soluble recombinant human PH20 (rHuPH20). The soluble PH20 is present in 2B2 cells at a copy number of approximately 206 copies/cell. Southern blot analysis of Spe I-, Xba I- and BamH I/Hind III-digested genomic 2B2 cell DNA using a PH20-specific probe revealed the following restriction digest profile: one major hybridizing band of ˜7.7 kb and four minor hybridizing bands (˜13.9, ˜6.6, ˜5.7 and ˜4.6 kb) with DNA digested with Spe I; one major hybridizing band of ˜5.0 kb and two minor hybridizing bands (˜13.9 and ˜6.5 kb) with DNA digested with Xba I; and one single hybridizing band of ˜1.4 kb observed using 2B2 DNA digested with BamH I/Hind III. Sequence analysis of the mRNA transcript indicated that the derived cDNA (SEQ ID NO:56) was identical to the reference sequence (SEQ ID NO:49) except for one base pair difference at position 1131, which was observed to be a thymidine (T) instead of the expected cytosine (C). This is a silent mutation, with no effect on the amino acid sequence.

### Example 3

### Production and Purification of Recombinant Human PH20 (rHuPH20)

### A. Production of Gen2-Soluble PH20 in 300 L Bioreactor Cell Culture

A vial of HZ24-2B2 cells (Example 2B) was thawed and expanded from shaker flasks through 36 L spinner flasks in CD-CHO media (Invitrogen, Carlsbad, Calif.) supplemented with 20 μM methotrexate and GlutaMAX-1™ (Invitrogen). Briefly, a vial of cells was thawed in a 37° C. water bath, media was added, and the cells were centrifuged. The cells were re-suspended in a 125 mL shake flask with 20 mL of fresh media and placed in a 37° C., 7% CO₂ incubator. The cells were expanded up to 40 mL in the 125 mL shake flask. When the cell density reached greater than 1.5×10⁶ cells/mL, the culture was expanded into a 125 mL spinner flask in a 100 mL culture volume. The flask was incubated at 37° C., 7% CO₂. When the cell density reached greater than 1.5×10⁶ cells/mL, the culture was expanded into a 250 mL spinner flask in 200 mL culture volume, and the flask was incubated at 37° C., 7% CO₂. When the cell density reached greater than 1.5×10⁶ cells/mL, the culture was expanded into a 1 L spinner flask in 800 mL culture volume and incubated at 37° C., 7% CO₂. When the cell density reached greater than 1.5×10⁶ cells/mL the culture was expanded into a 6 L spinner flask in 5000 mL culture volume and incubated at 37° C., 7% CO₂. When the cell density reached greater than 1.5×106 cells/mL the culture was expanded into a 36 L spinner flask in 32 L culture volume and incubated at 37° C., 7% CO₂.

A 400 L reactor was sterilized and 230 mL of CD-CHO media was added. Before use, the reactor was checked for contamination. Approximately 30 L cells were transferred from the 36 L spinner flasks to the 400 L bioreactor (Braun) at an inoculation density of 4.0×10⁵ viable cells per mL and a total volume of 260 L. Parameters were: temperature set point, 37° C.; impeller speed, 40-55 RPM; vessel pressure, 3 psi; air sparge, 0.5-1.5 L/min.; air overlay, 3 L/min. The reactor was sampled daily for cell counts, pH verification, media analysis, protein production and retention. Also, during the run, nutrient feeds were added. At 120 hrs (day 5), 10.4 L of Feed #1 Medium (4×CD-CHO+33 g/L glucose+160 mL/L Glutamax-1™+83 mL/L Yeastolate+33 mg/L rHulnsulin) was added. At 168 hours (day 7), 10.8 L of Feed #2 Medium (2×CD-CHO+33 g/L glucose+80 mL/L Glutamax-1™+167 mL/L Yeastolate+0.92 g/L sodium butyrate) was added, and culture temperature was changed to 36.5° C. At 216 hours (day 9), 10.8 L of Feed #3 Medium (1×CD-CHO+50 g/L Glucose+50 mL/L Glutamax-1™+250 mL/L Yeastolate+1.80 g/L sodium butyrate) was added, and culture temperature was changed to 36° C. At 264 hours (day 11), 10.8 L of Feed #4 Medium (lx CD-CHO+33 g/L glucose+33 mL/L Glutamax-1™+250 mL/L Yeastolate+0.92 g/L sodium butyrate) was added, and culture temperature was changed to 35.5° C. The addition of the feed media was observed to dramatically enhance the production of soluble rHuPH20 in the final stages of production. The reactor was harvested at 14 or 15 days or when the viability of the cells dropped below 40%. The process resulted in a final productivity of 17,000 Units/mL, with a maximal cell density of 12 million cells/mL. At harvest, the culture was sampled for mycoplasma, bioburden, endotoxin and viral in vitro and in vivo, Transmission Electron Microscopy (TEM) and enzyme activity.

The culture was pumped by a peristaltic pump through four Millistak filtration system modules (Millipore) in parallel, each containing a layer of diatomaceous earth graded to 4-8 μm and a layer of diatomaceous earth graded to 1.4-1.1 μm, followed by a cellulose membrane, then through a second single Millistak filtration system (Millipore) containing a layer of diatomaceous earth graded to 0.4-0.11 μm and a layer of diatomaceous earth graded to <0.1 μm, followed by a cellulose membrane, and then through a 0.22 μm final filter into a sterile single-use flexible bag with a 350 L capacity. The harvested cell culture fluid was supplemented with 10 mM EDTA and 10 mM Tris to a pH of 7.5. The culture was concentrated 10× with a tangential flow filtration (TFF) apparatus using four Sartoslice TFF 30 kDa molecular weight cut-off (MWCO) polyether sulfone (PES) filters (Sartorius), followed by a 10× buffer exchange with 10 mM Tris, 20 mM Na₂SO₄, pH 7.5, through a 0.22 μm final filter into a 50 L sterile storage bag.

The concentrated, diafiltered harvest was inactivated for virus. Prior to viral inactivation, a solution of 10% Triton X-100, 3% tri (n-butyl) phosphate (TNBP) was prepared. The concentrated, diafiltered harvest was exposed to 1% Triton X-100, 0.3% TNBP for 1 hour in a 36 L glass reaction vessel immediately prior to purification on the Q column.

### B. Purification of Gent-Soluble PH20

A Q Sepharose (Pharmacia) ion exchange column (9 L resin, H=29 cm, D=20 cm) was prepared. Wash samples were collected for a determination of pH, conductivity and endotoxin (LAL) assay. The column was equilibrated with 5 column volumes of 10 mM Tris, 20 mM Na₂SO₄, pH 7.5. Following viral inactivation, the concentrated, diafiltered harvest (Example 3A) was loaded onto the Q column at a flow rate of 100 cm/hr. The column was washed with 5 column volumes of 10 mM Tris, 20 mM Na₂SO₄, pH 7.5 and 10 mM Hepes, 50 mM NaCl, pH 7.0. The protein was eluted with 10 mM Hepes, 400 mM NaCl, pH 7.0, through a 0.22 μm final filter into sterile bag. The eluate sample was tested for bioburden, protein concentration and hyaluronidase activity. A₂₈₀ absorbance readings were taken at the beginning and end of the exchange.

Phenyl-Sepharose (Pharmacia) hydrophobic interaction chromatography was next performed. A phenyl-Sepharose (PS) column (19-21 L resin, H=29 cm, D=30 cm) was prepared. The wash was collected and sampled for pH, conductivity and endotoxin (LAL assay). The column was equilibrated with 5 column volumes of 5 mM potassium phosphate, 0.5 M ammonium sulfate, and 0.1 mM CaCl₂, pH 7.0. The protein eluate from the Q Sepharose column was supplemented with 2M ammonium sulfate, 1 M potassium phosphate and 1 M CaCl₂ stock solutions to yield final concentrations of 5 mM, 0.5 M and 0.1 mM, respectively. The protein was loaded onto the PS column at a flow rate of 100 cm/hr and the column flow-through collected. The column was washed with 5 mM potassium phosphate, 0.5 M ammonium sulfate and 0.1 mM CaCl₂, pH 7.0 at 100 cm/hr, and the wash was added to the collected flow-through. Combined with the column wash, the flow-through was passed through a 0.22 μm final filter into a sterile bag. The flow-through was sampled for bioburden, protein concentration and enzyme activity.

An aminophenyl boronate column (ProMedics) was prepared. The wash was collected and sampled for pH, conductivity and endotoxin (LAL assay). The column was equilibrated with 5 column volumes of 5 mM potassium phosphate and 0.5 M ammonium sulfate. The PS flow-through containing purified protein was loaded onto the aminophenyl boronate column at a flow rate of 100 cm/hr. The column was first washed with 5 mM potassium phosphate and 0.5 M ammonium sulfate, pH 7.0. The column was then washed with 20 mM bicine and 0.5 M ammonium sulfate, pH 9.0. The column was further washed with 20 mM bicine and 100 mM sodium chloride, pH 9.0. The protein was eluted with 50 mM Hepes, 100 mM NaCl, pH 6.9, and passed through a sterile filter into a sterile bag. The eluted sample was tested for bioburden, protein concentration and enzyme activity.

The hydroxyapatite (HAP) column (Biorad) was prepared. The wash was collected and tested for pH, conductivity and endotoxin (LAL assay). The column was equilibrated with 5 mM potassium phosphate, 100 mM NaCl and 0.1 mM CaCl₂, pH 7.0. The aminophenyl boronate-purified protein was supplemented to final concentrations of 5 mM potassium phosphate and 0.1 mM CaCl₂ and loaded onto the HAP column at a flow rate of 100 cm/hr. The column was washed with 5 mM potassium phosphate, pH 7, 100 mM NaCl and 0.1 mM CaCl₂. The column was next washed with 10 mM potassium phosphate, pH 7, 100 mM NaCl, and 0.1 mM CaCl₂. The protein was eluted with 70 mM potassium phosphate, pH 7.0, and passed through a 0.22 μm sterile filter into a sterile bag. The eluted sample was tested for bioburden, protein concentration and enzyme activity.

The HAP purified protein was then passed through a viral removal filter. The sterilized Viosart filter (Sartorius) was first prepared by washing with 2 L of 70 mM potassium phosphate, pH 7.0. Before use, the filtered buffer was sampled for pH and conductivity. The HAP-purified protein was pumped via a peristaltic pump through the 20 nM viral removal filter. The filtered protein in 70 mM potassium phosphate, pH 7.0, was passed through a 0.22 μm final filter into a sterile bag. The viral filtered sample was tested for protein concentration, enzyme activity, oligosaccharide, monosaccharide and sialic acid profiling. The sample also was tested for process related impurities.

### Example 4

### Determination of Hyaluronidase Activity of PH20

Hyaluronidase activity of soluble PH20 in samples such as cell cultures, plasma, purification fractions and purified solutions was determined using either a turbidimetric assay, which is based on the formation of an insoluble precipitate when hyaluronic acid binds with serum albumin, or a biotinylated-hyaluronic acid substrate assay, which measures the amount of enzymatically active rHuPH20 or PEGPH20 by the digestion of biotinylated hyaluronic acid (b-HA) substrate non-covalently bound to plastic, multi-well microtiter plates.

### A. Microturbidity Assay

Hyaluronidase activity of soluble rHuPH20 was measured by incubating soluble rHuPH20 with sodium hyaluronate (hyaluronic acid) for a set period of time (10 minutes) and then the undigested sodium hyaluronate was precipitated by the addition of acidified serum albumin. The turbidity of the resulting sample was measured at 640 nm after a 30 minute development period. The decrease in turbidity resulting from enzyme activity on the sodium hyaluronate substrate is a measure of the soluble rHuPH20 hyaluronidase activity. The method was performed using a calibration curve generated with dilutions of a soluble rHuPH20 assay working reference standard, and sample activity measurements were made relative to this calibration curve.

Dilutions of the sample were prepared in Enzyme Diluent Solutions. The Enzyme Diluent Solution was prepared by dissolving 33.0±0.05 mg of hydrolyzed gelatin in 25.0 mL of 50 mM PIPES reaction buffer (140 mM NaCl, 50 mM PIPES, pH 5.5) and 25.0 mL of sterile water for injection (SWFI), diluting 0.2 mL of 25% Buminate solution into the mixture, and vortexing for 30 seconds. This was performed within 2 hours of use and stored on ice until needed. The samples were diluted to an estimated 1-2 U/mL. Generally, the maximum dilution per step did not exceed 1:100 and the initial sample size for the first dilution was not less than 20 μL. The minimum sample volumes needed to perform the assay were as follows: in-process samples, FPLC fractions: 80 μL; tissue culture supernatants: 1 mL; concentrated material: 80 μL; purified or final step material: 80 μL. The dilutions were made in triplicate in a low protein binding 96-well plate, and 30 μL of each dilution was transferred to Optilux black/clear bottom plates (BD BioSciences).

Dilutions of known, soluble rHuPH20 with a concentration of 2.5 U/mL were prepared in Enzyme Diluent Solution to generate a standard curve and added to the Optilux plate in triplicate. The dilutions included 0 U/mL, 0.25 U/mL, 0.5 U/mL, 1.0 U/mL, 1.5 U/mL, 2.0 U/mL, and 2.5 U/mL. “Reagent blank” wells that contained 60 μL of Enzyme Diluent Solution were included in the plate as a negative control. The plate was then covered and warmed on a heat block for 5 minutes at 37° C. The cover was removed and the plate was shaken for 10 seconds. After shaking, the plate was returned to the heat block and the MULTIDROP 384 Liquid Handling Device was primed with the warm 0.25 mg/mL sodium hyaluronate solution (prepared by dissolving 100 mg of sodium hyaluronate (LifeCore Biomedical) in 20.0 mL of SWFI. This was mixed by gently rotating and/or rocking at 2-8° C. for 2-4 hours, or until completely dissolved). The reaction plate was transferred to the MULTIDROP 384 and the reaction was initiated by pressing the start key to dispense 30 μL sodium hyaluronate into each well. The plate was then removed from the MULTIDROP 384 and shaken for 10 seconds before being transferred to a heat block with the plate cover replaced. The plate was incubated at 37° C. for 10 minutes.

The MULTIDROP 384 was prepared to stop the reaction by priming the machine with Serum Working Solution (25 mL of Serum Stock Solution (1 volume of Horse Serum (Sigma) was diluted with 9 volumes of 500 mM acetate buffer solution and the pH was adjusted to 3.1 with hydrochloric acid) in 75 mL of 500 mM acetate buffer solution) and changing the volume setting to 240 μL. The plate was removed from the heat block and placed onto the MULTIDROP 384, and 240 μL of Serum Working Solution was dispensed into the wells. The plate was removed and shaken on a plate reader for 10 seconds. After a further 15 minutes, the turbidity of the samples was measured at 640 nm and the hyaluronidase activity (in U/mL) of each sample was determined by fitting to the standard curve.

Specific activity (Units/mg) was calculated by dividing the hyaluronidase activity (U/mL) by the protein concentration (mg/mL).

### B. Biotinylated Hyaluronan Assay

The biotinylated-hyaluronic acid assay measures the amount of enzymatically active rHuPH20 or PEGPH20 in biological samples by the digestion of a large molecular weight (˜1.2 megadaltons) biotinylated-hyaluronic acid (b-HA) substrate non-covalently bound to plastic, multi-well microtiter plates. The rHuPH20 or PEGPH20 in standards and samples were allowed to incubate in a plate coated with b-HA at 37° C. After a series of washes, remaining uncleaved/bound b-HA was treated with streptavidin horseradish peroxidase conjugate (SA-HRP). Reaction between immobilized SA-HRP and the chromogenic substrate, 3,3′,5,5′-tetramethylbenzidine (TMB), produces a blue colored solution. After stopping the reaction with acid, formation of the soluble yellow reaction product was determined by reading the absorbance at 450 nm using a microtiter plate spectrophotometer. The decrease in absorbance at 450 nm resulting from enzyme activity on the biotinylated-hyaluronic acid (b-HA) substrate is a measure of the soluble rHuPH20 hyaluronidase activity. The method was performed using a calibration curve generated with dilutions of a soluble rHuPH20 or PEGPH20 reference standard, and sample activity measurements were made relative to this calibration curve.

Dilutions of the sample and calibrator were prepared in Assay Diluent. The Assay Diluent was prepared by adding 1% v/v pooled plasma (from the appropriate species) to 0.1% (w/v) BSA in HEPES, pH 7.4. This was prepared daily and stored at 2-8° C. Depending upon the species type, as well as the anticipated hyaluronidase level, single or multiple dilutions were prepared to ensure at least one sample dilution would fall within the range of the calibration curve. To guide the selection of test sample dilution(s), information known about the dose of hyaluronidase administered, the route of administration, approximate plasma volume of the species and the time point were used to estimate the hyaluronidase activity levels. Each sample dilution was mixed as it was prepared by brief pulse-vortexing, and pipet tips were changed in between each dilution. In general, the dilutions began with an initial 50 or 100-fold dilution, followed by additional serial dilutions. A seven-point calibration curve of rHuPH20 or PEGPH20 (depending upon the treatment administered) was prepared ranging in concentration from 0.004 to 3.0 U/mL for rHuPH20 and from 0.037 to 27 U/mL for PEGPH20. One-hundred microliters (100 μL) of each test sample dilution and calibration curve point was applied to triplicate wells of a 96-well microtiter plate (Immulon 4HBX, Thermo) that had been previously coated with 100 μL per well of b-HA at 0.1 mg/mL and blocked with 250 μL of 1.0% (w/v) bovine serum albumin in PBS. Plate(s) were covered with an adhesive plate seal and incubated at 37° C. for approximately 90 minutes. At the end of the incubation period, the adhesive seal was removed from the plate, samples were aspirated, and the plate was washed five times with 300 μL per well Wash Buffer (10 mM phosphate buffer, 2.7 mM potassium chloride, 137 mM sodium chloride, pH 7.4, with 0.05% (v/v) Tween 20, PBST) using an automated plate washer (BioTek ELx405 Select CW, Program ‘4HBX1’). One hundred microliters of streptavidin-HRP conjugate Working Solution (Streptavidin-HRP conjugate (1:5,000 v/v) in 20 mM Tris-HCl, 137 mM sodium chloride, 0.025% (v/v) Tween 20, 0.1% (w/v) bovine serum albumin) was added per well. The plate was sealed and allowed to incubate at ambient temperature for approximately 60 minutes, protected from light and without shaking. At the end of the incubation period, the adhesive seal was removed from the plate, samples were aspirated and the plate washed five times with 300 μL per well Wash Buffer as described above. TMB solution (at ambient temperature) was added to each well and allowed to incubate, protected from light, for approximately five minutes at room temperature. TMB Stop Solution (KPL, Catalog #50-85-06) was then added as 100 μL per well. The absorbance of each well at 450 nm was determined using a microtiter plate spectrophotometer. The response of the calibration curve on each plate was modeled using a 4-parameter logistic curve fit. The hyaluronidase activity of each unknown was calculated by interpolation from the calibration curve, corrected for sample dilution factor, and reported in U/mL.

### Example 5

### Preparation of PEGylated rHuPH20

In this example, rHuPH20 was PEGylated by reaction of the enzyme with linear N-hydroxysuccinimidyl ester of methoxy poly(ethylene glycol) butanoic acid (MBA30K).

### A. Preparation of MBA30K

In order to generate PEGPH20, rHuPH20 (which is approximately 60 KDa in size) was covalently conjugated to a linear N-hydroxysuccinimidyl ester of methoxy poly(ethylene glycol) butanoic acid (MBA30K), having an approximate molecular weight of 30 kDa. The structure of MBA30K is shown below, where n≈681:

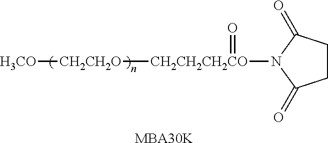

Methods used to prepare the MBA30K that was used to PEGylate rHuPH20 are described, for example, in U.S. Pat. No. 5,672,662. Briefly, the MBA30K was made according to the following procedure:

A solution of ethyl malonate (2 equivalents) dissolved in dioxane was added dropwise to a solution of sodium hydride (2 equivalents) and toluene under a nitrogen atmosphere, followed by the addition of a solution of mPEG methane sulfonate (1 equivalent, MW 30 kDa, Shearwater) in toluene. The resulting mixture was refluxed for approximately 18 hours. The reaction mixture was then concentrated to half its original volume and extracted with a 10% aqueous NaCl solution followed by extraction with a 1% aqueous hydrochloric acid solution. The aqueous extracts were combined and extracted with dichloromethane (3×), the organic layer was dried with magnesium sulfate, then filtered and evaporated to dryness. The resulting residue was dissolved in 1N sodium hydroxide containing sodium chloride and the mixture was stirred for 1 hour. The pH of the mixture was adjusted to approximately 3 by addition of 6N hydrochloric acid.

The mixture was extracted with dichloromethane (2×), the organic layer was collected and dried over magnesium sulfate, filtered, concentrated, and poured into cold diethyl ether. The precipitate was collected by filtration and dried under vacuum. The resulting compound was dissolved in dioxane, refluxed for 8 hours, and then concentrated to dryness. The resulting residue was dissolved in water and extracted with dichloromethane (2×), dried over magnesium sulfate, concentrated by rotary evaporation, and poured into cold diethyl ether. The precipitate was collected by filtration and dried under vacuum.

The resulting compound (1 equivalent) was dissolved in dichloromethane and N-hydroxysuccinimide (2.1 equivalents) was added. The solution was cooled to 0° C. and a solution of dicyclohexylcarbodiimide (2.1 equivalents) in dichloromethane was added dropwise. The solution was stirred at room temperature for approximately 18 hours. The reaction mixture was filtered, concentrated and precipitated in diethyl ether. The precipitate was collected by filtration and dried under vacuum to afford the powder MBA30K which was then frozen at −15° C.

B. Conjugation of MBA30K to rHuPH20

To make the PEGPH20, MBA30K was coupled to the amino group(s) of rHuPH20 by covalent conjugation, providing stable amide bonds between rHuPH20 and mPEG, as shown below, where n≈681.

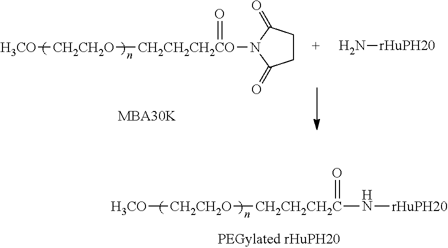

Prior to conjugation, the rHuPH20-purified bulk protein made in Example 3B was concentrated to 10 mg/mL using a 10 kDa polyethersulfone (PES) tangential flow filtration (TFF) cassette (Sartorius) with a 0.2 m² filtration area, and buffer exchanged against 70 mM potassium phosphate at pH 7.2. The concentrated protein was then stored at 2-8° C. until use.

To conjugate the rHuPH20, the MBA30K was thawed at room temperature in the dark for not longer than 2 hours. Depending on the batch size, a sterile 3″ stir bar was placed into a 1 or 3 L Erlenmeyer flask and buffer exchanged rHuPH20 protein was added. Five grams of dry MBA30K powder per gram of rHuPH20 (10:1 molar ratio of MBA30K: rHuPH20) was added to the flask under a vacuum hood and the mixture was mixed for 10 minutes or until the MBA30K was completely dissolved. The stir rate was set such that vortexing occurred without foaming.

The solution was then filtered under a class 100 hood by pumping the solution, via peristaltic pump, through a 0.22 μm polystyrene, cellulose acetate filter capsule (Corning 50 mL Tubetop filter) into a new 1 or 3 L Erlenmeyer flask containing a sterile 3″ stir bar. The volume of the PEGPH20 reaction mixture was determined by mass (1 g/mL density) and the 0.22 μm filter used for filtration was examined in a post-use integrity test.

The mixture was then placed on a stir plate at 2-8° C. and mixed for 20±1 hours in the dark. The stir rate was again set such that vortexing occurred without foaming. The entire Erlenmeyer was wrapped in foil to protect the solution from light. After mixing, the reaction was quenched by adding 1M glycine to a final concentration of 25 mM. Samples were removed from the container to test pH and conductivity. The pH and conductivity were then adjusted by adding to a solution of 5 mM Tris Base (5.65 L/L) and 5 mM Tris, 10 mM NaCl, pH 8.0 (13.35 L/L) to proceed with Q Sepharose purification.

A QFF Sepharose (GE Healthcare) ion exchange column (Height=21.5-24.0 cm, Diameter=20 cm) was prepared by equilibration with 5 column volumes (36 L) of 5 mM Tris, 10 mM NaCl, pH 8.0. The conjugated product was loaded onto the QFF column at a flow rate of 95 cm/hr. The column was then washed with 11 L of equilibration buffer (5 mM Tris, 10 mM NaCl, pH 8.0) at a flow rate of 95 cm/hr followed by a wash with 25 L of equilibration buffer at a flow rate of 268 cm/hr. The protein product was then eluted with 5 mM Tris, 130 mM NaCl, pH 8.0, at a flow rate of 268 cm/hr. The resulting purified PEGPH20 was concentrated to 3.5 mg/mL, using a 30 kDa polyethersulfone (PES) tangential flow filtration (TFF) cassette (Sartorius) with a 0.2 m² filtration area, and buffer exchanged against 10 mM Histidine, 130 mM NaCl at pH 6.5. The resulting material was tested for enzyme activity as described in Example 4. The PEGylated rHuPH20 material, at a concentration of 3.5 mg/mL (final enzyme activity 140,000 U/mL), was filled, in 3 mL volumes, into 5 mL glass vials with siliconized bromobutyl rubber stoppers and aluminum flip-off seals, and frozen (frozen overnight in a −80° C. freezer, then put in a −20° C. freezer for longer storage). The PEGylated rHuPH20 contained approximately 4.5 moles of PEG per mole of rHuPH20.

Following conjugation and purification, the pegylated rHuPH20 (PEGPH20) enzyme retained at least 25% of the initial specific activity (30,000 units/mg versus ˜100,000 units/mg; final PEGPH20 specific activity >38,000 units/mg).

### Example 6

### Effect of PEGPH20 on Hyaluronan (HA) Content in Skeletal Muscle of Mice

PEGPH20 was administered to mice to assess the effect of HA levels in muscle extracellular matrix (ECM). High-fat (HF) diet-induced obese (D10) mice and chow fed (control) mice were generated as described above in Example 1. After treatment of mice with PEGPH20 as indicated below, HA content was measured by immunohistochemistry as described in Example 1. Plasma hyaluronidase was measured by the method described above in Example 4.

### A. Chow Fed (Control) Mice

Sixteen week old chow fed male C57/BL6J mice received a tail vein intravenous (IV) injection of PEGPH20 (prepared according to Example 4 above) at 10 mg/kg body weight. The mice were sacrificed at either 4, 6, 12, 24, 48 or 72 hours after injection (n=3 per time point) and the HA content in muscle was measured by immunohistochemistry, as detailed above in Example 1. Plasma hyaluronidase activity also was measured in the same mice to determine the half-life of the PEGPH20.

The immunohistochemical stains showed that four hours after PEGPH20 injection, the HA in muscle decreased by 80% and that the level was maintained up to 72 hours after IV injection (data were normalized to t=0; p<0.05 vs. 0 hr). These results indicate that PEGPH20 is effective at lowering the HA content in muscle in normal mice.

The results also showed significant plasma hyaluronidase activity in mice that was measured to be approximately 5000 Units/mL at 4 hours after injection, and which steadily decreased over time. The detected activity in plasma collected 6 hours after injection was approximately 4000 U/mL, by 12 and 24 hours it was approximately 1500 U/mL, and at later time points was less than 1000 U/mL. These results showed that PEGPH20 in mouse plasma had a half-life of 5.1 hours.

### B. High Fat Diet-Induced Obese Mice

Fifteen week old DIO mice received injections of either vehicle (10 mM histidine, 130 nM NaCl, pH 6.5) or PEGPH20 at 10 mg/kg body weight intravenously once every 3 days for 24 days.

The HA content in muscle ECM was measured by immunohistochemistry, as detailed above in Example 1, and data were normalized to vehicle-treated HF fed mice. The immunohistochemical stains showed that repeated injections of PEGPH20 over a 24 day period decreased almost 90% of the HA in muscle (n=6; p<0.05 vs. vehicle HF). These results indicate that PEGPH20 is effective at lowering the HA content in muscle in diet-induced obese mice.

### Example 7

### Effect of PEGPH20 on Physical Activity and Energy Expenditure in Obese Mice

High-fat (HF) diet-induced obese (DIO) mice were generated as described above in Example 1. After one week of acclimation to the animal facility, 18 week old DIO mice (n=6 per group) received an IV tail vein injection once every 3 days for 21 days of: vehicle (10 mM histidine, 130 nM NaCl, pH 6.5); PEGPH20 at 10 mg/kg body weight; or PEGPH20 at dosages of either 0.001, 0.01, 0.1 or 1 mg/kg body weight.

Energy expenditure (EE) and physical activity of the mice were assessed by an Oxymax Deluxe System (Columbus Instruments, Columbus, Ohio). To measure energy expenditure, the rate of oxygen consumption (VO₂) and carbon dioxide production (VCO₂) were calculated and averaged over the light and dark cycles for each mouse and calculated as described by Bohlooly et al. (Diabetes 54:51-62 (2005)) using the equation: EE (Kcal/kg/hr)=(3.815+1.232×RER)×VO₂, where VO₂ is the volume of oxygen consumed per hour normalized to body weight and RER represents VCO₂/VO₂. Physical (ambulatory) activity was estimated as the number of infrared beams broken in both X and Y directions over the light and dark cycles.

Energy expenditure was measured for 2 or 3 days and 3 nights after the injection of vehicle or PEGPH20. EE was generally comparable during the light cycles for the first 2 or 3 days in vehicle-treated mice and mice treated with PEGPH20, though mice treated with higher dosages of PEGPH20 (e.g. 1 mg/kg and 10 mg/kg) exhibited slightly lower energy expenditure than mice treated with vehicle or lower levels of PEGPH20. EE measurements from the first 3 dark cycles showed that mice treated with higher levels of PEGPH20 (e.g. 1 mg/kg and 10 mg/kg) had slightly decreased EE during the first 2 dark cycles as compared to vehicle-treated mice and mice treated with lower doses of PEGPH20, but was comparable by the third dark cycle.

Energy expenditure was measured again after the 7^{th} injection of either 0.001, 0.01, 0.1 or 1 mg/kg body weight PEGPH20, with mice receiving 0.001 mg/kg PEGPH20 expending significantly more energy than the other mice on Nights 18 and 19 and Day 18. Mice treated with 0.1 mg/kg PEGPH20 exhibited an increase in energy expenditure on Day 19 as compared to the other groups of mice. By Night 20, all groups of mice (vehicle-treated and PEGPH20-treated) did not display a significant difference in energy expenditure. These results demonstrate that chronic treatment of PEGPH20 was well tolerated in mice, without overt adverse effects on energy expenditure.

During the first light and dark cycle, there was a significant decrease in physical (ambulatory) activity in mice treated with higher amounts of PEGPH20 (0.1 mg/kg, 1 mg/kg, and 10 mg/kg PEGPH20) as compared to vehicle-treated mice and mice treated with lower amounts of PEGPH20. After the second dark cycle however, only mice treated with the highest dose of PEGPH20 (10 mg/kg) exhibited decreased physical activity.

### Example 8

### Effect of PEGPH20 on Body Weight and Fat Mass in Obese Mice

High fat (HF) diet-induced obese (D10) mice were treated as described in Example 7 above with vehicle only or varying amounts of PEGPH20. In some cases, mice were treated up to 24 days or 27 days with PEGPH20. Body weight, food consumption and fat and lean mass were measured and monitored over the course of the experiment. Mice treated with the highest doses of PEGPH20 (0.1 mg/kg, 1 mg/kg and 10 mg/kg), exhibited an initial decrease in body weight that was sustained during the course of PEGPH20 treatment compared to vehicle, which was the result of an increase in lean mass, but a decrease in fat mass. For example, initially (i.e. three days after the first injection), mice treated with 10 mg/kg PEGPH20 experienced approximately a 10% decrease in body weight and fat mass, but no change in lean mass (n=10; p<0.05 vs. vehicle; p<0.05 vs. Day 0). However, this decrease in body weight and fat mass was determined to be due to decreased food consumption (˜50%) rather than from PEGPH20, Subsequent injections did not cause any further changes in body weight and the mice treated with 10 mg/kg PEGPH20 subsequently gained weight at the same rate as the vehicle-treated mice (0.12±0.03 g/day in vehicle mice vs. 0.09±0.03 g/day in 10 mg/kg PEGPH20 mice), though the fat mass remained decreased after all 8 injections. Mice treated with 0.01 mg/kg PEGPH20 also experienced a decrease in fat mass, but no significant overall decrease in whole body weight. These results demonstrate that chronic treatment of PEGPH20 was well tolerated in mice, without overt adverse effects on body weight and fat mass.

### Example 9

### Effect of Acute PEGPH20 Treatment on Insulin Resistance in High Fat Diet-Induced Obese Mice

High fat (HF) diet-induced obese (DIO) mice were treated intravenously with vehicle only or 10 mg/mL PEGPH20, Seventy-two (72) hours after administration of the intravenous bolus, hyperinsulinemic euglycemic clamp was performed on mice after acute treatment with 10 mg/kg PEGPH20 to assess insulin sensitivity. Basal fasting glucose, plasma insulin and non-esterified fatty acid concentrations were measured in 5 hour-fasted DIO mice to assess insulin sensitivity.

A hyperinsulinemic euglycemic clamp was performed using the following general procedure (Ayala et al. (2006) Diabetes 55:390-397):

Five days prior to the study, catheters were implanted in a carotid artery and a jugular vein of the mice for sampling and infusions. [3-³1-1]glucose (2.4 μCi) was primed and continuously infused for a 90 minute equilibration period (0.04 μCi/min) and a 2 hour clamp period (0.12 μCi/min). Baseline blood or plasma parameters were determined in blood samples collected at −15 and −5 minutes. At t=0, insulin infusion (4 mU/kg/min) was started and continued for 165 minutes. Blood glucose was clamped at ˜150 mg/dL using a variable glucose infusion. Mice received heparinized saline-washed erythrocytes from donors at 5 μL/min to prevent a fall in hematocrit. The hyperinsulinemic euglycemic clamp was validated by assessment of blood glucose over time. Blood glucose was monitored every 10 minutes and the GIR was adjusted as needed. Blood was taken at 80-120 minutes for the determination of [3-³H]glucose. Clamp insulin was determined at t=100 and 120 minutes. At 120 minutes, 13 μCi of 2[¹⁴C]deoxyglucose ([¹⁴C]2DG) was administered as an intravenous bolus. Blood was taken at 2-35 minutes after injection of the bolus for the determination of [¹⁴C]2DG. After the last sample, mice were anesthetized and tissues were collected.

[3-³H]glucose and [¹⁴C]2DG levels were measured in order to determine the effects of insulin, at euglycemia, on glucose metabolism in individual tissues in vivo. Plasma [3-3H]glucose and [¹⁴C]2DG radioactivity were determined by liquid scintillation counting (Packard TRI-CARB 2900TR) with Ultima Gold (Packard) as scintillant. Glucose appearance (Rₐ) and disappearance (R_{d}) rates (i.e. glucose flux) were determined using non-steady state equations (Steele et al. (1956) Am. J. Physiol. 187:15-24). Endogenous glucose production (endoRₐ) was determined by subtracting the GIR from total Rₐ. The glucose metabolic index (R_{g}) was calculated as described in Kraegen et al. (1985) Am. J. Physiol. 248:E353-E362.

Body weight, blood glucose concentration, plasma insulin concentration and plasma non-esterified fatty acid (NEFA) concentration were measured and are shown below in Table 5.

TABLE 5
Basal and clamp characteristics of DIO mice after acute vehicle or
PEGPH20 treatment

| | Vehicle | PEGPH20 |
|---|---|---|
| n | 4 | 5 |
| Weight (g) | 35 ± 1.5 | 34 ± 1.9 |
| Blood glucose (mg/dL) | | |
| Basal | 125 ± 9 | 119 ± 8 |
| Clamp^{§} | 154 ± 3 | 151 ± 2 |
| Plasma insulin (ng/mL) | | |
| Basal | 3.1 ± 1.0 | 3.6 ± 0.9 |
| Clamp^{§} | 7.5 ± 0.4 | 7.6 ± 0.7 |
| Plasma NEFA (mM) | | |
| Basal | 0.90 ± 0.13 | 0.62 ± 0.04 |
| Clamp^{§} | 0.14 ± 0.01 | 0.14 ± 0.04 |

^{§}Data are presented as the average of values obtained from 80-120 minutes of the hyperinsulinemic euglycemic clamp
All data are expressed as mean ± SEM

The results showed that one injection of PEGPH20 did not affect basal concentrations of fasting glucose, plasma insulin, or NEFA concentration. Glucose concentration was clamped at 150 mg/dL in both groups of mice during the hyperinsulinemic euglycemic clamp. Plasma insulin was equivalently elevated and NEFA levels were decreased to a similar extent in both groups of mice during the hyperinsulinemic euglycemic clamp.

Glucose infusion rate (GIR), endogenous glucose production (endoRₐ) and disappearance rate (R_{d}), and muscle glucose uptake index (R_{g}) in the gastrocnemius and superficial vastus lateralus (SVL) were determined as described above (n=4-5). The GIR required to maintain euglycemia during the hyperinsulinemic euglycemic clamp was comparable between the two groups of mice, with an initial GIR of ˜25 mg/kg/min and rising to ˜42 mg/kg/min by the end of the hyperinsulinemic euglycemic clamp (t=120 min). This indicates that insulin sensitivity was unaffected by acute PEGPH20 treatment. Likewise, R_{g} in the gastrocnemius and SVL were not statistically different between the two groups. EndoRₐ and R_{d} were similar between the two groups of mice at basal state and during the hyperinsulinemic euglycemic clamp. These data indicate that one injection of 10 mg/kg PEGPH20 did not affect insulin action in DIO mice.

### Example 10

### Effect of Chronic PEGPH20 Treatment on Insulin Resistance in High Fat Diet-Induced Obese Mice

High fat (HF) diet-induced obese (DIO) mice generated as described in Example 1 were treated intravenously with vehicle only, 10 mg/kg PEGPH20 or varying amounts of PEGPH20 (0.001, 0.01, 0.1 or 1 mg/kg) every 3 days for 24 days. Three days after the last treatment, a hyperinsulinemic euglycemic clamp was performed on mice after chronic treatment with PEGPH20 to assess insulin sensitivity, following the technique described in Example 9. Basal fasting glucose, plasma insulin and non-esterified fatty acid concentrations were measured. Glucose infusion rate (GIR), endogenous glucose production (endoRₐ) and disappearance rate (R_{d}), and muscle glucose uptake index (R_{g}) in the gastrocnemius and superficial vastus lateralus (SVL) were determined as described above in Example 9.

Glucose concentration was clamped at 150 mg/dL in all groups of mice during the hyperinsulinemic euglycemic clamp. Consistent with lower basal plasma insulin, clamp insulin concentration in the chronic 10 mg/kg PEGPH20-treated mice was 50% lower than what was seen in chronic vehicle-treated mice during the hyperinsulinemic euglycemic clamp. Plasma NEFA was also lower during the hyperinsulinemic euglycemic clamp in the 10 mg/kg PEGPH20-treated mice relative to vehicle-treated mice. The decreased plasma NEFA concentration during the hyperinsulinemic euglycemic clamp indicates increased insulin-induced suppression of lipolysis, consistent with the broad interstitial presence of HA. Chronic treatment with 10 mg/kg PEGPH20 also did not affect the basal concentrations of arterial glucose or NEFA in the mice. In contrast, basal arterial insulin was decreased by 50% in the 10 mg/kg PEGPH20 mice as compared to the vehicle mice.

Despite the lowered insulin concentration during the hyperinsulinemic euglycemic clamp, all PEGPH20-treated mice, with the exception of mice treated with the lowest dosage of PEGPH20 (0.001 mg/kg), required higher GIR to maintain euglycemia as compared to mice treated with vehicle. This shows that PEGPH20 improves insulin action in a dose-dependent manner, with less effects observed at the lowest concentration tested.

Basal endoRₐ and R_{d} were similar in all groups of mice, except for mice treated with 1 mg/kg PEGPH20, which exhibited a slightly higher basal endoRₐ and R_{d}. During the hyperinsulinemic euglycemic clamp, however, endoRₐ was lower in the mice treated with the highest dosages of PEGPH20 (1 mg/kg and 10 mg/kg) as compared to the other mice. R_{d} during the hyperinsulinemic euglycemic clamp was equivalently increased in all groups of mice relative to baseline. R_{g} in the gastrocnemius and SVL was also increased in the 10 mg/kg PEGPH20-treated mice when compared to chronic vehicle-treated mice. The increased glucose metabolic index (R_{g}) and glucose fluxes indicate that chronic treatment with PEGPH20 is dose-dependent whereby higher amounts of PEGPH20 improve insulin sensitivity in DIO mice while lower dose PEGPH20 treatment have a lesser effect or no effect on insulin action in DIO mice.

Insulin-induced suppression of liver glucose production, as calculated by: (Basal endoRₐ-Clamp endoRₐ)/Basal endoRₐ×100%, was also increased.

### Example 11

### Effect of Chronic PEGPH20 Treatment on Muscle Insulin-Mediated Signaling

In animals treated as described in Example 10, the effect of PEGPH20 on Akt signaling was assessed to determine if the improved insulin action in mice treated with 10 mg/kg PEGPH20 was a consequence of increased expression of Akt rather than improved activation of the existing signaling components. Protein expression of Akt and phosphorylated Akt was assessed. At the conclusion of the hyperinsulinemic euglycemic clamp, gastrocnemius muscle was collected and Western Blotting was performed as follows:

Gastrocnemius was homogenized in buffer containing 50 mM Tris-HCl (pH7.5), 1 mM EDTA, 1 mM EGTA, 10% glycerol, 1% Triton X-100, 1 mM DTT, 1 mM PMSF, 5 μg/mL protease inhibitor, 50 mM NaF, and 5 mM sodium pyrophosphate, and centrifuged at 13,000 rpm for 20 minutes at 4° C. 40 μg of the supernatant was applied to 4-12% SDS-PAGE gel. Phosphorylated and total Akt/PKB were probed using phospho-Akt(Ser473) and Akt antibodies (Cell Signaling, Danvers, Mass.).

The increased glucose flux and muscle R_{g} during the hyperinsulinemic euglycemic clamp was consistent with increased muscle insulin signaling after the clamp. Phosphorylation of Akt as well as total Akt were increased in gastrocnemius of chronic PEGPH20-treated DIO mice, indicative of improved insulin signaling, while the ratio of the two was not increased (n=3-4; p<0.05 vs. vehicle; data were normalized to vehicle). The fact that both phosphorylated and total Akt were increased, but the ratio of the two was not, shows that improved insulin action was a consequence of increased expression of Akt and not improved activation of the existing signaling components.

### Example 12

### Effect of Chronic PEGPH20 Treatment on Collagen Expression in Muscle and Muscle Vascularization

Increased skeletal muscle collagen levels are observed in diet-induced insulin resistant mice, with an increase in vascularization occurring following reversal of muscle insulin resistance (Kang et al. (2011) Diabetes 60:416-426). In animals treated as described in Example 10, the effect of chronic PEGPH20 treatment on collagen IV (ColIV) levels and vascularization in skeletal muscle was assessed by immunohistochemistry. Immunohistochemistry was performed as described above in Example 1 on the gastrocnemius muscles collected at the end of the hyperinsulinemic euglycemic clamp, using anti-ColIV (Abcam, Cambridge, Mass.) as the primary antibody. Immunostaining of CD31 was carried out using anti-CD31 (BD Biosciences). ColIV expression was measured by the integrated intensity of staining and muscle vascularity was determined by counting CD31-positive structures.

Percent cardiac output to muscle (i.e. skeletal muscle blood flow) was also assessed, using the procedure in Lee-Young et al. (2009) J. Biol. Chem. 284:23925-23934. Briefly, following the final arterial blood sample of the hyperinsulinemic euglycemic clamp, 50 μL of yellow DYE-TRAK® microspheres (15 μm; Triton Technology, Inc., San Diego, Calif.) were injected into the carotid artery, followed by a small volume of saline. Gastrocnemius was then immediately collected. Microspheres in the tissues were isolated and N,N-dimethylformamide was used to elute the fluorescent dye from the microspheres. The optical density of the N,N-dimethylformamide solution was determined at a wave length of 450 nm. Adequacy of microsphere mixing was assumed if the concentrations of microspheres in the right and left kidney were within 10%.

Immunohistochemistry showed that chronic PEGPH20 treatment in DIO mice did not affect the expression of ColIV in muscle as compared to vehicle-treated mice (n=6-8; vehicle=1.00±0.31; chronic PEGPH20=1.11±0.15; data normalized to vehicle). Consistent with the observation that reversal of muscle insulin resistance during a HF diet feeding is associated with increased muscle vascularization, the results showed that increased muscle insulin action in the PEGPH20-treated DIO mice was associated with an increase in the expression of the vascular marker, CD31 (n=6-8; vehicle=1.00±0.08; chronic PEGPH20=1.31±0.07; p<0.05 vs. vehicle; data normalized to vehicle). Increased muscle vascularization in the PEGPH20-treated mice was also consistent with a 1.5 fold increase in the fraction of cardiac output going to skeletal muscle in the PEGPH20-treated DIO mice (n=4-8; p<0.05 vs. vehicle; data normalized to vehicle).

### Example 13

### Effect of Chronic PEGPH20 Treatment on Glucose Uptake in Isolated Muscle

In animals treated as described in Example 10, the effect of PEGPH20 glucose uptake was assessed in isolated muscles to further define whether the effects of chronic PEGPH20 treatment were due to sensitization of muscle to the direct actions of insulin. On day 27 (3 days after the final injection), mice chronically treated with vehicle or 10 mg/kg PEGPH20 for 24 days were anesthetized and soleus and extensor digitorum longus (EDL) muscles were excised. Glucose uptake in isolated muscles was measured as follows (Jorgensen et al. (2004) J. Biol. Chem. 279:1070-1079): Soleus and EDL muscles were incubated in Krebs Henseleit bicarbonate buffer containing 1.2 mM KH₂PO₄, 25 mM NaHCO₃, 118.5 mM NaCl, 4.7 mM KCl, 2.5 mM CaCl₂, 1.2 mM MgSO₄, pH 7.4 supplemented with 8 mM mannitol, 2 mM pyruvate and 0.01% bovine serum albumin at 30° C. under continuous gassing with 95% O₂ and 5% CO₂. After 10 minutes of basal incubation, muscles were stimulated with or without insulin at 10 mU/mL for 30 minutes. [³H]2DG uptake was measured by adding cold 2DG (1 mM), [³H]2DG (0.25 μCi/mL) and D-[¹⁴C]mannitol (0.16 Ci/mL). After 10 minutes of incubation, muscles were washed and homogenized. Radioactivity in the supernatant was determined by liquid scintillation counting.

In vitro [³H]2DG uptake in isolated soleus was higher at basal state in the PEGPH20-treated mice vs. vehicle-treated mice (n=7-9; p<0.05 vs. vehicle). Ten minutes of insulin stimulation at 10 mU/mL increased [³]2DG uptake in both groups (PEGPH20 and vehicle) by similar increments/fold changes (n=7-9; p<0.05 vs. vehicle and p<0.05 vs. basal). [³H]2DG uptake was the same in the EDL in both basal and insulin-stimulated states in the PEGPH20-treated mice and vehicle-treated mice (n=7-9; p<0.05 vs. vehicle and p<0.05 vs. basal).

Insulin-stimulated muscle glucose uptake in isolated muscles in vitro was not affected by chronic PEGPH20 treatment, though increased muscle insulin action with PEGPH20 treatment was observed in vivo. Increased muscle insulin action in vivo was associated with increased muscle vascularization and a greater percent cardiac output going to muscle. Isolated muscle in vitro lacks the intact physiological interactions between muscle fibers and surrounding matrix and capillaries. The improved vascular access of hormones and glucose in vivo is likely responsible for the increased insulin action. When the interaction between muscle and surrounding matrix and capillaries is disassociated, muscle insulin action is no longer increased by chronic PEGPH20. An increase in basal muscle glucose uptake was observed in isolated soleus, demonstrating that chronic PEGPH20 treatment has an impact on muscle cells, especially the slow-twitch fibers, as EDL muscle, which is primarily composed of fast-twitch fibers, was not affected.

### Example 14

### Effect of Chronic PEGPH20 Treatment on Adipocyte Cell Size and Gene Expression of Macrophage Markers in Adipose Tissue

Insulin responsiveness of adipose tissue is dependent upon adipose cell size, with larger adipose cells observed in obese subjects, thus resulting in lower insulin sensitivity in the tissue (Salmis et al. (1968) J. Clin. Invest. 47:153-165). In animals treated as described in Example 10, the adipose tissue of the DIO mice treated with 10 mg/kg PEGPH20 or vehicle-treatment were stained with Toluidine Blue 0 according to manufacturer's instruction (Newcomer Supply, Middleton, Wis.). Adipocyte diameter and distribution range was determined by manual measurements of the diameters of at least 100 adipocytes from each mouse (n=5 mice per group). The results indicated that adipocyte cell size was significantly reduced in the adipose tissue of mice treated with PEGPH20 as compared to mice treated with vehicle.

Many inflammation- and macrophage-specific genes are highly upregulated in white adipose tissue in high-fat diet-induced obese mice (Xu et al. (2003) J. Clin. Invest. 112:1821-1830). To determine the effect of chronic treatment of PEGPH20 on the expression of inflammation and macrophage-specific genes in HF DIO mice, RNA was isolated from 100 mg of perigonadal adipose tissue using the RNeasy minikit from Qiagen (Valencia, Calif.). cDNA was synthesized using the iScript cDNA synthesis kit from BioRad (Hercules, Calif.). cDNA was diluted 1:2 or 1:10 and then used for real-time PCR analysis on a BioRad iQ5 machine. Tagman gene expression assays were purchased from Applied Biosystems (Foster City, Calif.) for the following genes: F4/80 (Mm00802529_m1), CD11c (Mm00498698_m1), TNFa (Mm00443258_m1), IL-12 (Mm00434165_m1), IL-1b (Mm01336189_m1), CCL2 (Mm00441242_m1), Mel1 (Mm00546124_m1), IL-10 (Mm99999062_m1), CD 163 (Mm00474091_m1). Quantification of 18S (4352930E) was performed for each sample, and final relative concentration was determined by comparing each gene of interest to 18S using the delta delta CT method (Livak et al. (2001) Methods. 25: 402-408).

The results showed that while there was no significant difference in total gene expression of macrophage (Mθ) markers F4/80 and CD11c, there was a statistical difference in several M1 (classically activated macrophages) and M2 (alternatively activated macrophages) markers. Expression of M1 markers IL-12, IL-1b and CCL2, and M2 markers IL-10, CD163 and Mgl1 was reduced in PEGPH20-treated mice as compared to vehicle-treated mice. Expression of M1 marker TNF was not significantly reduced in PEGPH20-treated mice.

Since modifications will be apparent to those of skill in this art, it is intended that this invention be limited only by the scope of the appended claims.

1. A method of reducing or ameliorating insulin resistance in a subject, comprising:
administering to a subject who exhibits a symptom of insulin resistance, a hyaluronan-degrading enzyme, wherein:
the hyaluronan-degrading enzyme is conjugated to a polymer; and
the hyaluronan-degrading enzyme is administered in an amount sufficient to remove or degrade skeletal muscle-associated hyaluronan, whereby hyaluronan is degraded, resulting in a decrease or elimination of insulin resistance.

2. The method of claim 1, wherein the hyaluronan-degrading enzyme is administered a plurality of times.

3. The method of claim 1, wherein the hyaluronan-degrading enzyme is administered at a predetermined frequency.

4. The method of claim 1, wherein the hyaluronan-degrading enzyme is administered until a symptom of insulin resistance is reduced by at least 5%, 10%, 20%, 30%, 40%, 50%, 60%, 70%, 80% or more compared to the symptom prior to treatment.

5. The method of claim 3, wherein the hyaluronan-degrading enzyme is administered to the subject at a frequency of at least once a month.

6. The method of claim 3, wherein the hyaluronan-degrading enzyme is administered at a frequency of at least twice a month.

7. The method of claim 3, wherein the hyaluronan-degrading enzyme is administered at a frequency of at least twice a week, three times a week, four times a week, five times a week, six times a week or seven times a week.

8. The method of claim 3, wherein the hyaluronan-degrading enzyme is administered at a frequency of at least twice a week.

9. The method of claim 3, wherein the hyaluronan-degrading enzyme is administered at the frequency of administration for a predetermined time in a cycle of administration.

10. The method of claim 9, wherein the predetermined time is at least or is one week, two weeks, three weeks, four weeks, two months, three months, four months, five months, six months, seven months, eight months, nine months, 10 months, 11 months or 12 months.

11. The method of claim 9, wherein the cycle of administration is repeated a plurality of times.

12. The method of claim 1, wherein the subject is obese.

13. The method of claim 12, wherein the subject has a body mass index (BMI) of greater than 30 kg/m².

14. The method of claim 12, wherein:
the subject has a BMI of greater than 32 kg/m², 33 kg/m², 34 kg/m², 35 kg/m², 40 kg/m², 45 kg/m² or greater; or
the subject has a BMI of between about 30 kg/m² to 50 kg/m²; 30 kg/m² to 40 kg/m²; 35 kg/m² to 50 kg/m²; 30 kg/m² to 35 kg/m²; or 35 kg/m² to 40 kg/m².

15. The method of claim 1, wherein the subject exhibits one or more of hyperglycemia, dyslipidemia, hyperlipidemia, or hyperinsulinemia.

16. The method of claim 1, wherein the subject exhibits insulin resistance as assessed by fasting insulin levels, a glucose tolerance test (GTT), a hyperinsulinemic euglycemic clamp, an insulin tolerance test (ITT), an insulin sensitivity test (IST), continuous infusion of glucose with model assessment (CIGMA), homeostatic model assessment (HOMA-IR), quantitative insulin sensitivity check index (QUICKI), McAuley's index, Matsuda index, Belfiore index, Cederholm index, Gutt index, Avignon index and Stumboll index.

17. The method of claim 1, wherein:
the subject exhibits insulin resistance as assessed by fasting insulin levels; and
the fasting insulin levels are greater than 10 Units/mL, 15 Units/mL, 20 Units/mL, 25 Units/mL, 30 Units/mL, 35 Units/mL, 40 Units/mL or higher.

18. The method of claim 1, wherein:
the subject exhibits insulin resistance as assessed by a method selected from among:
a) homeostasis model assessment (HOMA-IR); wherein the HOMA-IR is greater than 2.2, 2.3, 2.5, 2.6, 2.7, 2.8, 2.9, 3.0, 3.5, 4.0, 5.0 or 6.0; or.
b) hyperinsulinemic euglycemic clamp with insulin infused at a rate of about or at least 40 mU/m²/min, wherein the subject exhibits a glucose disposal rate (GDR) of less than 7.5 mg/kg per minute, less than 7.0 mg/kg per minute, 6.5 mg/kg per minute, 6.0 mg/kg per minute, 5.5 mg/kg per minute, 5.0 mg/kg per minute, 4.5 mg/kg per minute, 4.0 mg/kg per minute, 3.5 mg/kg per minute, 3.0 mg/kg per minute or lower; or
c) quantitative insulin sensitivity check index (QUICKI), wherein the QUICKI is less than 0.360, 0.359, 0.358, 0.357, 0.355, 0.350, 0.345, 0.340, 0.335, 0.330, 0.325, 0.320, 0.315 or 0.310; or
d) McAuley's index, wherein the McAuley's index is less than 6.0, 5.9, 5.8, 5.7, 5.6, 5.5, 5.0, 4.5, 4.0, 3.5 or 3.0.

19. The method of claim 1, comprising: prior to administering the hyaluronan-degrading enzyme to the subject, selecting a subject for treatment having increased or accumulated skeletal muscle-associated hyaluronan.

20. The method of claim 19, wherein selecting a subject comprises:
a) measuring the expression or level of a hyaluronan-associated marker in a sample from a subject;
b) comparing the expression or level of the marker to expression or level of the same marker in a control sample or standard; and
c) if the marker is changed compared to the control sample or standard, selecting the subject for treatment with the hyaluronan-degrading enzyme.

21. The method of claim 20, wherein the expression or level of the marker is elevated or increased compared to the control sample or standard.

22. The method of claim 21, wherein the expression or level of the marker is elevated or increased at least 0.5-fold, 1-fold, 2-fold, 2.5-fold, 3.0-fold, 4.0-fold, 5.0-fold or greater.

23. The method of claim 20, wherein the expression or level of the marker is decreased compared to the control sample or standard.

24. The method of claim 23, wherein the expression or level of the marker is decreased at least 0.5-fold, 1-fold, 2-fold, 2.5-fold, 3.0-fold, 4.0-fold, 5.0-fold or greater.

25. The method of claim 20, wherein the hyaluronan-associated marker is selected from among hyaluronan (HA), a hyaluronidase, a hyaluronan synthase, interstitial fluid pressure, vascular volume and water content.

26. The method of claim 25, wherein the marker is hyaluronan (HA), and the hyaluronan is detected using an anti-HA antibody or an HA-binding protein.

27. The method of claim 25, wherein the marker is a hyaluronan synthase that is hyaluronan synthase 2 (HAS2).

28. The method of claim 20, wherein the sample is a fluid sample selected from among blood (plasma), urine and saliva.

29. The method of claim 20, wherein the sample is a sample from skeletal muscle.

30. The method of claim 20, wherein the control sample in b) is selected from among:
a) an analogous sample from another subject that is a normal subject or a subject known to express low hyaluronan in the sample; and
b) a cell line.

31. The method of claim 1, wherein the subject has a disease or condition associated with insulin resistance that is selected from among obesity, type 2 diabetes, hypertension, dyslipidemia, coronary artery disease, atherosclerosis and polycystic ovarian syndrome.

32. The method of claim 1, wherein reducing or ameliorating insulin resistance prevents or ameliorates a disease or condition associated with insulin resistance selected from among obesity, type 2 diabetes, hypertension, dyslipidemia, coronary artery disease, atherosclerosis and polycystic ovarian syndrome.

33. The method of claim 1, wherein the hyaluronan-degrading enzyme is a hyaluronidase.

34. The method of claim 33, wherein the hyaluronidase is a PH20 or a truncated form thereof that lacks a C-terminal glycosylphosphatidylinositol (GPI) attachment site or a portion of the GPI attachment site.

35. The method of claim 33, wherein the hyaluronidase is a PH20 that is a human.

36. The method of claim 35, wherein:
the hyaluronan-degrading enzyme is a C-terminally truncated PH20 that does not consist of the full-length sequence of PH20 set forth in SEQ ID NO:1; and
the truncated PH20 comprises at least the sequence of amino acids 36-464 of SEQ ID NO:1, or comprises a sequence of amino acids that has at least 85% sequence identity to the sequence of amino acids that contains at least amino acids 36-464 of SEQ ID NO:1 and retains hyaluronidase activity.

37. The method of claim 36, wherein the hyaluronan-degrading enzyme is a truncated PH20 that comprises a sequence of amino acids that has at least 86%, 87%, 88%, 89%, 90%, 91%, 92%, 93%, 94%, 95%, 96%, 97%, 98% or 99% sequence identity to a sequence of amino acids that contains at least amino acids 36-464 of SEQ ID NO:1 and retains hyaluronidase activity.

38. The method of claim 36, wherein:
the PH20 comprises a sequence of amino acids that contains a C-terminal truncation at or after amino acid position 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO:1, or is a variant thereof that exhibits at least 85% sequence identity to a sequence of amino acids that contains a C-terminal truncation after amino acid position 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO:1 and retains hyaluronidase activity; or
the PH20 consists of the sequence of amino acids set forth in any of SEQ ID NOS: 4-9, 47, 48, 150-170, 183-189, or a sequence of amino acids that exhibits at least 85% sequence identity to any of SEQ ID NOS: 4-9, 47, 48, 150-170, 183-189.

39. The method of claim 38, wherein:
the PH20 comprises a sequence of amino acids that has at least 86%, 87%, 88%, 89%, 90%, 91%, 92%, 93%, 94%, 95%, 96%, 97%, 98% or 99% sequence identity to a sequence of amino acids that contains a C-terminal truncation after amino acid position 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO:1 and retains hyaluronidase activity; or
the PH20 consists of a sequence of amino acids that exhibits at least 86%, 87%, 88%, 89%, 90%, 91%, 92%, 93%, 94%, 95%, 96%, 97%, 98% or 99% sequence identity to the sequence of amino acids set forth in any of SEQ ID NOS: 4-9, 47, 48, 150-170, 183-189.

40. The method of claim 35, wherein the PH20 is a composition designated rHuPH20 and contains a polypeptide that consists of amino acids 36-482 of SEQ ID NO:1.

41. The method of claim 1, wherein the polymer is a polyalkylene glycol, dextran, pullulan or cellulose.

42. The method of claim 41, wherein the polyalkylene glycol is selected from among polyethylene glycols (PEG) or methoxypolyethylene glycols (mPEG).

43. The method of claim 40, wherein the polymer is a polyethylene glycol (PEG).

44. The method of claim 41, wherein the polymer is a PEG, and the PEG is a branched or linear PEG.

45. The method of claim 1, wherein the hyaluronan-degrading enzyme is administered in a dosage range amount of between or about between 0.001 μg/kg to 25 mg/kg (of the subject).

46. The method of claim 45, wherein the hyaluronan-degrading enzyme is administered in a dosage range amount of between or about between 0.5 μg/kg to 10 μg/kg.

47. The method of claim 1, wherein the hyaluronan-degrading enzyme is administered orally, intravenously (IV), subcutaneously, intramuscularly, intra-tumorally, intradermally, topically, transdermally, rectally, intrathecally or sub-epidermally.

48. The method of claim 1, further comprising administering a corticosteroid in an amount sufficient to ameliorate any side-effects from administration of the hyaluronan-degrading enzyme.

49. The method of claim 48, wherein the corticosteroid is a glucocorticoid.

50. The method of claim 49, wherein the glucocorticoid is selected from among cortisones, dexamethasones, hydrocortisones, methylprednisolones, prednisolones and prednisones.

51. The method of claim 48, wherein the corticosteroid is administered prior to, concurrent with, intermittently with or subsequent to administration of hyaluronan-degrading enzyme.

52. The method of claim 1, further comprising effecting treatment by administering another agent or providing another treatment for reducing or ameliorating insulin resistance.

53. The method of claim 52, wherein the subject has a disease or condition associated with insulin resistance, and the method further comprises administering another agent or treatment for treating or ameliorating the disease or condition associated with insulin resistance.

54. The method of claim 53, wherein the disease or condition associated with insulin resistance is selected from among type 2 diabetes, cardiovascular disease or polycystic syndrome.

55. The method of claim 1, wherein the subject is a human.
